(19) United States
(12) Patent Application Publication
(10) Pub. No.: US 2018/0172668 A1
(43) Pub. Date: Jun. 21, 2018

(54) ENGINEERED MODEL OF FIBROTIC DISEASES

(71) Applicant: Colorado State University Research Foundation, Fort Collins, CO (US)

(72) Inventors: Salman R. Khetani, Chicago, IL (US); Matthew D. Davidson, Chicago, IL (US)

(73) Assignee: Colorado State University Research Foundation, Fort Collins, CO (US)

(21) Appl. No.: 15/738,878

(22) PCT Filed: Jun. 23, 2016
(86) PCT No.: PCT/US16/39068

§ 371 Date: Dec. 21, 2017

Related U.S. Application Data
(60) Provisional application No. 62/183,555, filed on Jun. 23, 2015.

(51) Int. Cl. G01N 33/50; A61K 35/33; A61K 35/15; G01N 33/12; A61K 35/407; A61K 35/44; C12N 5/071

(52) U.S. Cl. CPC G01N 33/5014; A61K 35/33; A61K 35/15; G01N 33/12; A61K 35/407; C12N 2502/14; C12N 5/067; A61K 2300/00; C12N 2502/13; C12N 2502/1171; A61K 35/44

(57) ABSTRACT

The present disclosure provides co-cultures of a population of hepatocytes, at least one non-parenchymal cell population, and a population of hepatic stellate cells in vitro, methods of preparing the co-cultures, and methods of using the co-cultures for high throughput screening and evaluation of drug candidates. The hepatocytes co-culture system provides an in vitro model in which both cell viability and phenotype are maintained for extended periods relative to conventional monoculture.

### CROSS REFERENCE TO RELATED APPLICATIONS

This application claims the benefit of U.S. Provisional Application No. 62/183,555, filed Jun. 23, 2015, the disclosure of which is hereby incorporated by reference in its entirety.

### GOVERNMENTAL RIGHTS

This invention was made with government support under 1R03EB019184-01 awarded by the National Institutes of Health and grant 1351909 awarded by the National Science Foundation. The government has certain rights in the invention.

### FIELD OF THE INVENTION

The disclosure relates to in vitro cultures of hepatocytes, and in particular co-cultures systems including hepatocytes, non-parenchymal cells, and hepatic stellate cells, and the use of the co-cultures in developing and screening drugs.

### BACKGROUND

Chronic liver injury from several stimuli (e.g., chronic hepatitis B/C infection, alcohol abuse, non-alcoholic fatty liver disease and drugs) can cause fibrosis in the liver, which results from an imbalance between production and degradation of extracellular matrix (“ECM”). Hepatic stellate cells (“HSCs”) play a prominent role in fibrosis by differentiating into myofibroblast-like cells that scar the liver by depositing excessive ECM. Fibrosis can lead to cirrhosis (the most advanced stage of fibrosis) and ultimately hepatocellular carcinoma (“HCC”), which is the third most common cause of cancer-related death. Cirrhosis is the strongest predisposing factor for the development of HCC and thus reducing or reversing fibrosis/cirrhosis could potentially be beneficial for lowering the incidence of HCC. Several molecular targets have been identified as anti-fibrotic agents, some of which have entered early-phase clinical studies, but progress has been stymied due to the lack of sensitive and specific biomarkers that indicate fibrosis progression or reversal in humans.

Understanding and treating human liver fibrosis requires complementing in vivo animal studies with in vitro models of the human liver given differences in liver pathways (e.g., drug metabolism enzymes or DMEs) and disease progression across different species.

Co-cultures of primary human hepatocytes (“PHHs”) and primary HSCs can potentially be useful as a model of the early stages of human liver fibrosis. A few investigators have created such co-cultures in vitro, and they have found that HSCs can induce phenotypic functions in PHHs relative to declining pure PHH monolayers. However, PHH-HSC co-cultures are functionally unstable over time as PHHs still dedifferentiate towards a mesenchymal phenotype. In the absence of a stable and physiologically relevant model of PHH functions, it is not possible to ascertain whether a specific stromal cell type, such as activated HSCs, positively or negatively impacts the hepatic phenotype. Thus, with current in vitro approaches, it remains unclear whether PHHs in a fibrotic/cirrhotic liver (with activated and proliferating HSCs) are normally functioning or dysfunctional.

Furthermore, with the aforementioned “conventional” co-culture models, it is not possible to determine whether potential anti-fibrotic drugs can alleviate the symptoms of fibrosis or not. Based on the aforementioned state-of-the-art in PHH-HSC co-cultures, better controlled and stable models of PHH functions can serve to elucidate the role of heterotypic cell-cell interactions on phenotypic evolution of the key cell types of the liver, namely PHHs and HSCs. Furthermore, a physiologically relevant human liver model can help the process of screening for the efficacy and toxicity of drug candidates, such as those for fibrosis.

### SUMMARY

In one aspect, the present disclosure provides a composition comprising a population of hepatocytes, at least one non-parenchymal cell population, and a population of hepatic stellate cells (HSCs) in co-culture in vitro. The hepatocytes may be primary human hepatocytes. The hepatocytes may be derived from any mammalian pluripotent stem cells. Hepatocytes may be obtained from one or more human donors suffering from a disorder of the liver. The disorder of the liver may be Type-2 diabetes, metabolic syndrome, non-alcoholic fatty liver disease (NAFLD), non-alcoholic steatohepatitis (NASH), and cardiovascular disease. At least one of the non-parenchymal cell populations may comprise stromal cells. Stromal cells may comprise fibroblasts, fibroblast-derived cells, macrophages, endothelial cells, stellate cells, pericytes, inflammatory cells, cholangiocytes and other types of stromal cells, and combinations thereof. For example, stromal cells may comprise 3T3-J2 murine embryonic fibroblasts. The at least one non-parenchymal cell population may be obtained from an individual not suffering from a disorder of the liver. The at least one non-parenchymal cell population may be obtained from an individual suffering from a disorder of the liver. The HSCs may be primary HSCs from one or more donors. The HSCs may be an immortalized HSC line. The HSCs may also be cancerous HSCs or animal-derived HSCs. The HSCs may be obtained from an individual not suffering from a disorder of the liver. The HSCs may be obtained from an individual suffering from a disorder of the liver. The HSCs may be in an activated state. The HSCs may be in a deactivated/quiescent state. The composition may further comprise a culture substrate. The culture substrate may be hard or soft. A hard culture substrate may comprise a glass surface, a polystyrene surface, or a silicon surface. A soft culture substrate may comprise an extracellular matrix, extracellular matrix gel, or hydrogel. The hard culture substrate may have a hardness of more than 5 kPa to about 60 GPa. The soft culture substrate may have a hardness of about 0.15 KPa to about 5 KPa. A biopolymer scaffold may be disposed on the culture substrate.

In some aspects, the population of hepatocytes and non-parenchymal cells may be disposed in a micropattern on a culture substrate. In further aspects, the population of hepatocytes, non-parenchymal cells, and HSCs may be disposed in a micropattern on a culture substrate The micropattern may be a random or a predetermined two-dimensional pattern of multiple microdots, defined by a microdot diameter and an edge-to-edge spacing between each of any two neighboring microdots. For example, when the micropattern comprises a predetermined two-dimensional pattern of multiple microdots, each microdot may have a diameter of 10 μm to 1000 μm, and the edge-to-edge spacing between each microdot may be at least about 200 μm to about 1300 μm. The non-parenchymal cell population may occupy the inter-microdot space which is not occupied by the hepatocytes. The HSCs may occupy the inter-microdot space which is not occupied by the hepatocytes or may be dispersed throughout the co-culture.

In one aspect, the population of HSCs may be cultured on a layer of material comprising at least one extracellular matrix protein that is disposed on the population of hepatocytes and at least one non-parenchymal cell population.

In another aspect, the population of HSCs may be cultured on a substrate that is in fluid communication with the population of hepatocytes and at least one non-parenchymal cell population, and wherein the population of hepatic stellate cells is not in physical contact with the population of hepatocytes and at least one non-parenchymal cell population. The HSCs may be cultured on transwells. The transwells may be transferred to the co-culture of population of hepatocytes and at least one non-parenchymal cell population. The transwell may be absorbed with an extracellular matrix or extracellular matrix gels.

In another aspect, the present disclosure provides a composition comprising a population of hepatocytes and at least one non-parenchymal cell population in co-culture in vitro, and conditioned medium obtained from a population of activated hepatic stellate cells. The hepatocytes may be primary human hepatocytes. The hepatocytes may be derived from any mammalian pluripotent stem cells. Hepatocytes may be obtained from one or more human donors suffering from a disorder of the liver. The disorder of the liver may be Type-2 diabetes, metabolic syndrome, non-alcoholic fatty liver disease (NAFLD), non-alcoholic steatohepatitis (NASH), and cardiovascular disease. At least one of the non-parenchymal cell populations may comprise stromal cells. Stromal cells may comprise fibroblasts, fibroblast-derived cells, macrophages, endothelial cells, stellate cells, pericytes, inflammatory cells, cholangiocytes and other types of stromal cells, and combinations thereof. For example, stromal cells may comprise 3T3-J2 murine embryonic fibroblasts. The at least one non-parenchymal cell population may be obtained from an individual not suffering from a disorder of the liver. The at least one non-parenchymal cell population may be obtained from an individual suffering from a disorder of the liver. The conditioned medium is obtained from a population of activated hepatic stellate cells. The HSCs may be primary HSCs from one or more donors. The HSCs may be an immortalized HSC line. The HSCs may also be cancerous HSCs or animal-derived HSCs. The HSCs may be obtained from an individual not suffering from a disorder of the liver. The HSCs may be obtained from an individual suffering from a disorder of the liver. The HSCs may be cultured on a culture substrate. A hard culture substrate may comprise a glass surface, a polystyrene surface, or a silicon surface. A soft culture substrate may comprise an extracellular matrix, extracellular matrix gel, or hydrogel. The hard culture substrate may have a hardness of more than 5 kPa to about 60 GPa. The soft culture substrate may have a hardness of about 0.15 KPa to about 5 KPa. Similarly, the population of hepatocytes and at least one non-parenchymal cell population in co-culture may comprise a culture substrate. A biopolymer scaffold may be disposed on the culture substrate. In some aspects, the population of hepatocytes and non-parenchymal cells may be disposed in a micropattern the population of hepatocytes and non-parenchymal cells may be disposed in a micropattern on a culture substrate. The micropattern may be a random or a predetermined two-dimensional pattern of multiple microdots, defined by a microdot diameter and an edge-to-edge spacing between each of any two neighboring microdots. For example, when the micropattern comprises a predetermined two-dimensional pattern of multiple microdots, each microdot may have a diameter of 10 μm to 1000 μm, and the edge-to-edge spacing between each microdot may be at least about 200 μm to about 1300 μm. The non-parenchymal cell population may occupy the inter-microdot space which is not occupied by the hepatocytes. The HSCs may occupy the inter-microdot space which is not occupied by the hepatocytes or may be dispersed throughout the co-culture.

In another aspect, the present disclosure provides a method of culturing a population of hepatocytes in vitro comprising: co-culturing the population of hepatocytes with at least one non-parenchymal cell population and a population of hepatic stellate cells. The hepatocytes may be primary human hepatocytes. The hepatocytes may be derived from any mammalian pluripotent stem cells. Hepatocytes may be obtained from one or more human donors suffering from a disorder of the liver. The disorder of the liver may be Type-2 diabetes, metabolic syndrome, non-alcoholic fatty liver disease (NAFLD), non-alcoholic steatohepatitis (NASH), and cardiovascular disease. At least one of the non-parenchymal cell populations may comprise stromal cells. Stromal cells may comprise fibroblasts, fibroblast-derived cells, macrophages, endothelial cells, stellate cells, pericytes, inflammatory cells, cholangiocytes and other types of stromal cells, and combinations thereof. For example, stromal cells may comprise 3T3-J2 murine embryonic fibroblasts. The at least one non-parenchymal cell population may be obtained from an individual not suffering from a disorder of the liver. The at least one non-parenchymal cell population may be obtained from an individual suffering from a disorder of the liver. The HSCs may be primary HSCs from one or more donors. The HSCs may be an immortalized HSC line. The HSCs may also be cancerous HSCs or animal-derived HSCs. The HSCs may be obtained from an individual not suffering from a disorder of the liver. The HSCs may be obtained from an individual suffering from a disorder of the liver. The HSCs may be in an activated state. The HSCs may be in a deactivated/quiescent state. The composition may further comprise a culture substrate. The culture substrate may be hard or soft. A hard culture substrate may comprise a glass surface, a polystyrene surface, or a silicon surface. A soft culture substrate may comprise an extracellular matrix, extracellular matrix gel, or hydrogel. The hard culture substrate may have a hardness of more than 5 kPa to about 60 GPa. The soft culture substrate may have a hardness of about 0.15 KPa to about 5 KPa. A biopolymer scaffold may be disposed on the culture substrate. In some aspects, the population of hepatocytes and non-parenchymal cells may be disposed in a micropattern on a culture substrate. In further aspects, the population of hepatocytes, non-parenchymal cells, and HSCs may be disposed in a micropattern on a culture substrate The micropattern may be a random or a predetermined two-dimensional pattern of multiple microdots, defined by a microdot diameter and an edge-to-edge spacing between each of any two neighboring microdots. For example, when the micropattern comprises a predetermined two-dimensional pattern of multiple microdots, each microdot may have a diameter of 10 μm to 1000 μm, and the edge-to-edge spacing between each microdot may be at least about 200 μm to about 1300 μm. The non-parenchymal cell population may occupy the inter-microdot space which is not occupied by the hepatocytes. The HSCs may occupy the inter-microdot space which is not occupied by the hepatocytes or may be dispersed throughout the co-culture. In one aspect, the population of HSCs may be cultured on a layer of material comprising at least one extracellular matrix protein that is disposed on the population of hepatocytes and at least one non-parenchymal cell population. In another aspect, the population of HSCs may be cultured on a substrate that is in fluid communication with the population of hepatocytes and at least one non-parenchymal cell population, and wherein the population of hepatic stellate cells is not in physical contact with the population of hepatocytes and at least one non-parenchymal cell population. The HSCs may be cultured on transwells. The transwells may be transferred to the co-culture of population of hepatocytes and at least one non-parenchymal cell population. The transwell may be absorbed with an extracellular matrix or extracellular matrix gels.

In another aspect, the present disclosure provides a method of culturing a population of hepatocytes in vitro comprising: co-culturing the population of hepatocytes with at least one non-parenchymal cell population and with conditioned medium obtained from a population of activated HSCs in vitro. Co-culturing the population of hepatocytes with at least one non-parenchymal cell population conditioned medium obtained from a population of activated HSCs, may comprise: co-culturing the population of hepatocytes with at least one non-parenchymal cell population and providing conditioned medium obtained from a population of activated HSCs. The hepatocytes may be primary human hepatocytes. The hepatocytes may be derived from any mammalian pluripotent stem cells. Hepatocytes may be obtained from one or more human donors suffering from a disorder of the liver. The disorder of the liver may be Type-2 diabetes, metabolic syndrome, non-alcoholic fatty liver disease (NAFLD), non-alcoholic steatohepatitis (NASH), and cardiovascular disease. At least one of the non-parenchymal cell populations may comprise stromal cells. Stromal cells may comprise fibroblasts, fibroblast-derived cells, macrophages, endothelial cells, stellate cells, pericytes, inflammatory cells, cholangiocytes and other types of stromal cells, and combinations thereof. For example, stromal cells may comprise 3T3-J2 murine embryonic fibroblasts. The at least one non-parenchymal cell population may be obtained from an individual not suffering from a disorder of the liver. The at least one non-parenchymal cell population may be obtained from an individual suffering from a disorder of the liver. The conditioned medium is obtained from a population of activated hepatic stellate cells. The HSCs may be primary HSCs from one or more donors. The HSCs may be an immortalized HSC line. The HSCs may also be cancerous HSCs or animal-derived HSCs. The HSCs may be obtained from an individual not suffering from a disorder of the liver. The HSCs may be obtained from an individual suffering from a disorder of the liver. The HSCs may be cultured on a culture substrate. A hard culture substrate may comprise a glass surface, a polystyrene surface, or a silicon surface. A soft culture substrate may comprise an extracellular matrix, extracellular matrix gel, or hydrogel. The hard culture substrate may have a hardness of more than 5 kPa to about 60 GPa. The soft culture substrate may have a hardness of about 0.15 KPa to about 5 KPa. Similarly, the population of hepatocytes and at least one non-parenchymal cell population in co-culture may comprise a culture substrate. A biopolymer scaffold may be disposed on the culture substrate. In some aspects, the population of hepatocytes and non-parenchymal cells may be disposed in a micropattern the population of hepatocytes and non-parenchymal cells may be disposed in a micropattern on a culture substrate. The micropattern may be a random or a predetermined two-dimensional pattern of multiple microdots, defined by a microdot diameter and an edge-to-edge spacing between each of any two neighboring microdots. For example, when the micropattern comprises a predetermined two-dimensional pattern of multiple microdots, each microdot may have a diameter of 10 μm to 1000 μm, and the edge-to-edge spacing between each microdot may be at least about 200 μm to about 1300 μm. The non-parenchymal cell population may occupy the inter-microdot space which is not occupied by the hepatocytes. The HSCs may occupy the inter-microdot space which is not occupied by the hepatocytes or may be dispersed throughout the co-culture.

In another aspect, the present disclosure provides a method of determining the toxicity of a test compound, the method comprising: obtaining a co-culture of a population of hepatocytes, at least one non-parenchymal cell population, and a population of hepatic stellate cells in vitro; contacting the co-culture with the test compound; maintaining the co-culture for a time and under conditions sufficient to allow an effect of the test compound on the hepatocytes; and measuring at least one indicator of hepatic function in the hepatocytes to obtain a test measurement, or applying hepatocyte imaging technology (HIAT) to the hepatocytes to obtain a test image; and comparing the test measurement to a control measurement from the hepatocytes before contact with the test compound, or the test image to a control image of the hepatocytes before contact with the test compound, wherein a difference between the test and control is indicative of hepatotoxicity of the test compound. In another aspect, at least one indicator of hepatic stellate cell function is measured as well. The at least one indicator of cell function can be, for example, albumin production, urea production, ATP production, glutathione production, enzyme activity, lipid accumulation, liver gene expression or liver protein expression in the hepatocytes and/or HSC cells. The hepatocytes may be primary human hepatocytes. The hepatocytes may be derived from any mammalian pluripotent stem cells. Hepatocytes may be obtained from one or more human donors suffering from a disorder of the liver. The disorder of the liver may be Type-2 diabetes, metabolic syndrome, non-alcoholic fatty liver disease (NAFLD), non-alcoholic steatohepatitis (NASH), and cardiovascular disease. At least one of the non-parenchymal cell populations may comprise stromal cells. Stromal cells may comprise fibroblasts, fibroblast-derived cells, macrophages, endothelial cells, stellate cells, pericytes, inflammatory cells, cholangiocytes and other types of stromal cells, and combinations thereof. For example, stromal cells may comprise 3T3-J2 murine embryonic fibroblasts. The at least one non-parenchymal cell population may be obtained from an individual not suffering from a disorder of the liver. The at least one non-parenchymal cell population may be obtained from an individual suffering from a disorder of the liver. The HSCs may be primary HSCs from one or more donors. The HSCs may be an immortalized HSC line. The HSCs may also be cancerous HSCs or animal-derived HSCs. The HSCs may be obtained from an individual not suffering from a disorder of the liver. The HSCs may be obtained from an individual suffering from a disorder of the liver. The HSCs may be in an activated state. The HSCs may be in a deactivated/quiescent state. The composition may further comprise a culture substrate. The culture substrate may be hard or soft. A hard culture substrate may comprise a glass surface, a polystyrene surface, or a silicon surface. A soft culture substrate may comprise an extracellular matrix, extracellular matrix gel, or hydrogel. The hard culture substrate may have a hardness of more than 5 kPa to about 60 GPa. The soft culture substrate may have a hardness of about 0.15 KPa to about 5 KPa. A biopolymer scaffold may be disposed on the culture substrate. In some aspects, the population of hepatocytes and non-parenchymal cells may be disposed in a micropattern on a culture substrate. In further aspects, the population of hepatocytes, non-parenchymal cells, and HSCs may be disposed in a micropattern on a culture substrate. The micropattern may be a random or a predetermined two-dimensional pattern of multiple microdots, defined by a microdot diameter and an edge-to-edge spacing between each of any two neighboring microdots. For example, when the micropattern comprises a predetermined two-dimensional pattern of multiple microdots, each microdot may have a diameter of 10 μm to 1000 μm, and the edge-to-edge spacing between each microdot may be at least about 200 μm to about 1300 μm. The non-parenchymal cell population may occupy the inter-microdot space which is not occupied by the hepatocytes. The HSCs may occupy the inter-microdot space which is not occupied by the hepatocytes or may be dispersed throughout the co-culture. In one aspect, the population of HSCs may be cultured on a layer of material comprising at least one extracellular matrix protein that is disposed on the population of hepatocytes and at least one non-parenchymal cell population. In another aspect, the population of HSCs may be cultured on a substrate that is in fluid communication with the population of hepatocytes and at least one non-parenchymal cell population, and wherein the population of hepatic stellate cells is not in physical contact with the population of hepatocytes and at least one non-parenchymal cell population. The HSCs may be cultured on transwells. The transwells may be transferred to the co-culture of population of hepatocytes and at least one non-parenchymal cell population. The transwell may be absorbed with an extracellular matrix or extracellular matrix gels.

In another aspect, the present disclosure provides a method of determining the hepatotoxicity of a test compound, the method comprising: co-culturing the population of hepatocytes with at least one non-parenchymal cell population and with conditioned medium obtained from a population of activated HSCs in vitro; contacting the co-culture with the test compound; maintaining the co-culture for a time and under conditions sufficient to allow an effect of the test compound on the hepatocytes; and measuring at least one indicator of hepatic function in the hepatocytes to obtain a test measurement, or applying hepatocyte imaging technology (HIAT) to the hepatocytes to obtain a test image; and comparing the test measurement to a control measurement from the hepatocytes before contact with the test compound, or the test image to a control image of the hepatocytes before contact with the test compound, wherein a difference between the test and control is indicative of hepatotoxicity of the test compound. The at least one indicator of hepatic function can be, for example, albumin production, urea production, ATP production, glutathione production, enzyme activity, lipid accumulation, liver gene expression or liver protein expression in the hepatocytes. The hepatocytes may be primary human hepatocytes. The hepatocytes may be primary human hepatocytes. The hepatocytes may be derived from any mammalian pluripotent stem cells. Hepatocytes may be obtained from one or more human donors suffering from a disorder of the liver. The disorder of the liver may be Type-2 diabetes, metabolic syndrome, non-alcoholic fatty liver disease (NAFLD), non-alcoholic steatohepatitis (NASH), and cardiovascular disease. At least one of the non-parenchymal cell populations may comprise stromal cells. Stromal cells may comprise fibroblasts, fibroblast-derived cells, macrophages, endothelial cells, stellate cells, pericytes, inflammatory cells, cholangiocytes and other types of stromal cells, and combinations thereof. For example, stromal cells may comprise 3T3-J2 murine embryonic fibroblasts. The at least one non-parenchymal cell population may be obtained from an individual not suffering from a disorder of the liver. The at least one non-parenchymal cell population may be obtained from an individual suffering from a disorder of the liver. The conditioned medium is obtained from a population of activated hepatic stellate cells. The HSCs may be primary HSCs from one or more donors. The HSCs may be an immortalized HSC line. The HSCs may also be cancerous HSCs or animal-derived HSCs. The HSCs may be obtained from an individual not suffering from a disorder of the liver. The HSCs may be obtained from an individual suffering from a disorder of the liver. The HSCs may be cultured on a culture substrate. A hard culture substrate may comprise a glass surface, a polystyrene surface, or a silicon surface. A soft culture substrate may comprise an extracellular matrix, extracellular matrix gel, or hydrogel. The hard culture substrate may have a hardness of more than 5 kPa to about 60 GPa. The soft culture substrate may have a hardness of about 0.15 KPa to about 5 KPa. Similarly, the population of hepatocytes and at least one non-parenchymal cell population in co-culture may comprise a culture substrate. A biopolymer scaffold may be disposed on the culture substrate. In some aspects, the population of hepatocytes and non-parenchymal cells may be disposed in a micropattern the population of hepatocytes and non-parenchymal cells may be disposed in a micropattern on a culture substrate. The micropattern may be a random or a predetermined two-dimensional pattern of multiple microdots, defined by a microdot diameter and an edge-to-edge spacing between each of any two neighboring microdots. For example, when the micropattern comprises a predetermined two-dimensional pattern of multiple microdots, each microdot may have a diameter of 10 μm to 1000 μm, and the edge-to-edge spacing between each microdot may be at least about 200 μm to about 1300 μm. The non-parenchymal cell population may occupy the inter-microdot space which is not occupied by the hepatocytes. The HSCs may occupy the inter-microdot space which is not occupied by the hepatocytes or may be dispersed throughout the co-culture.

In another aspect, the present disclosure provides a method of identifying a candidate test compound for use in treating a disorder of the liver, the method comprising: obtaining a co-culture of a population of hepatocytes, at least one non-parenchymal cell population, and a population of hepatic stellate cells in vitro; contacting the co-culture with the test compound; maintaining the co-culture for a time and under conditions sufficient to allow an effect of the test compound on the hepatocytes; and measuring at least one indicator of hepatic function in the hepatocytes to obtain a test measurement, or applying hepatocyte imaging technology (HIAT) to the hepatocytes to obtain a test image; and comparing the test measurement to a control measurement from the hepatocytes before contact with the test compound, or the test image to a control image of the hepatocytes before contact with the test compound, wherein a difference between the test and control is indicative of whether the test compound is a candidate for use in treating disorder of the liver. The disorder of the liver may be Type-2 diabetes, metabolic syndrome, non-alcoholic fatty liver disease (NAFLD), non-alcoholic steatohepatitis (NASH), and cardiovascular disease. In another aspect, at least one indicator of hepatic stellate cell function is measured as well. The at least one indicator of cell function may be, for example, albumin production, urea production, ATP production, enzyme activity, lipid accumulation, glutathione production, enzyme activity, lipid accumulation, liver gene expression, or liver protein expression in the hepatocytes and/or HSCs. The hepatocytes may be primary human hepatocytes. The hepatocytes may be derived from any mammalian pluripotent stem cells. Hepatocytes may be obtained from one or more human donors suffering from a disorder of the liver. The disorder of the liver may be Type-2 diabetes, metabolic syndrome, non-alcoholic fatty liver disease (NAFLD), non-alcoholic steatohepatitis (NASH), and cardiovascular disease. At least one of the non-parenchymal cell populations may comprise stromal cells. Stromal cells may comprise fibroblasts, fibroblast-derived cells, macrophages, endothelial cells, stellate cells, pericytes, inflammatory cells, cholangiocytes and other types of stromal cells, and combinations thereof. For example, stromal cells may comprise 3T3-J2 murine embryonic fibroblasts. The at least one non-parenchymal cell population may be obtained from an individual not suffering from a disorder of the liver. The at least one non-parenchymal cell population may be obtained from an individual suffering from a disorder of the liver. The HSCs may be primary HSCs from one or more donors. The HSCs may be an immortalized HSC line. The HSCs may also be cancerous HSCs or animal-derived HSCs. The HSCs may be obtained from an individual not suffering from a disorder of the liver. The HSCs may be obtained from an individual suffering from a disorder of the liver. The HSCs may be in an activated state. The HSCs may be in a deactivated/quiescent state. The composition may further comprise a culture substrate. The culture substrate may be hard or soft. A hard culture substrate may comprise a glass surface, a polystyrene surface, or a silicon surface. A soft culture substrate may comprise an extracellular matrix, extracellular matrix gel, or hydrogel. The hard culture substrate may have a hardness of more than 5 kPa to about 60 GPa. The soft culture substrate may have a hardness of about 0.15 KPa to about 5 KPa. A biopolymer scaffold may be disposed on the culture substrate. In some aspects, the population of hepatocytes and non-parenchymal cells may be disposed in a micropattern on a culture substrate. In further aspects, the population of hepatocytes, non-parenchymal cells, and HSCs may be disposed in a micropattern on a culture substrate The micropattern may be a random or a predetermined two-dimensional pattern of multiple microdots, defined by a microdot diameter and an edge-to-edge spacing between each of any two neighboring microdots. For example, when the micropattern comprises a predetermined two-dimensional pattern of multiple microdots, each microdot may have a diameter of 10 μm to 1000 μm, and the edge-to-edge spacing between each microdot may be at least about 200 μm to about 1300 μm. The non-parenchymal cell population may occupy the inter-microdot space which is not occupied by the hepatocytes. The HSCs may occupy the inter-microdot space which is not occupied by the hepatocytes or may be dispersed throughout the co-culture. In one aspect, the population of HSCs may be cultured on a layer of material comprising at least one extracellular matrix protein that is disposed on the population of hepatocytes and at least one non-parenchymal cell population. In another aspect, the population of HSCs may be cultured on a substrate that is in fluid communication with the population of hepatocytes and at least one non-parenchymal cell population, and wherein the population of hepatic stellate cells is not in physical contact with the population of hepatocytes and at least one non-parenchymal cell population. The HSCs may be cultured on transwells. The transwells may be transferred to the co-culture of population of hepatocytes and at least one non-parenchymal cell population. The transwell may be absorbed with an extracellular matrix or extracellular matrix gels.

In another aspect, the present disclosure provides a method of identifying a candidate test compound for use in treating a disorder of the liver, the method comprising: co-culturing the population of hepatocytes with at least one non-parenchymal cell population and with conditioned medium obtained from a population of activated HSCs in vitro; contacting the co-culture with the test compound; maintaining the co-culture for a time and under conditions sufficient to allow an effect of the test compound on the hepatocytes; and measuring at least one indicator of hepatic function in the hepatocytes to obtain a test measurement, or applying hepatocyte imaging technology (HIAT) to the hepatocytes to obtain a test image; and comparing the test measurement to a control measurement from the hepatocytes before contact with the test compound, or the test image to a control image of the hepatocytes before contact with the test compound, wherein a difference between the test and control is indicative of whether the test compound is a candidate for use in treating disorder of the liver. The disorder of the liver may be Type-2 diabetes, metabolic syndrome, non-alcoholic fatty liver disease (NAFLD), non-alcoholic steatohepatitis (NASH), and cardiovascular disease. The at least one indicator of hepatic function may be, for example, albumin production, urea production, ATP production, enzyme activity, lipid accumulation, glutathione production, liver gene expression, or liver protein expression in the hepatocytes. The hepatocytes may be primary human hepatocytes. The hepatocytes may be primary human hepatocytes. The hepatocytes may be derived from any mammalian pluripotent stem cells. Hepatocytes may be obtained from one or more human donors suffering from a disorder of the liver. The disorder of the liver may be Type-2 diabetes, metabolic syndrome, non-alcoholic fatty liver disease (NAFLD), non-alcoholic steatohepatitis (NASH), and cardiovascular disease. At least one of the non-parenchymal cell populations may comprise stromal cells. Stromal cells may comprise fibroblasts, fibroblast-derived cells, macrophages, endothelial cells, stellate cells, pericytes, inflammatory cells, cholangiocytes and other types of stromal cells, and combinations thereof. For example, stromal cells may comprise 3T3-J2 murine embryonic fibroblasts. The at least one non-parenchymal cell population may be obtained from an individual not suffering from a disorder of the liver. The at least one non-parenchymal cell population may be obtained from an individual suffering from a disorder of the liver. The conditioned medium is obtained from a population of activated hepatic stellate cells. The HSCs may be primary HSCs from one or more donors. The HSCs may be an immortalized HSC line. The HSCs may also be cancerous HSCs or animal-derived HSCs. The HSCs may be obtained from an individual not suffering from a disorder of the liver. The HSCs may be obtained from an individual suffering from a disorder of the liver. The HSCs may be cultured on a culture substrate. A hard culture substrate may comprise a glass surface, a polystyrene surface, or a silicon surface. A soft culture substrate may comprise an extracellular matrix, extracellular matrix gel, or hydrogel. The hard culture substrate may have a hardness of more than 5 kPa to about 60 GPa. The soft culture substrate may have a hardness of about 0.15 KPa to about 5 KPa. Similarly, the population of hepatocytes and at least one non-parenchymal cell population in co-culture may comprise a culture substrate. A biopolymer scaffold may be disposed on the culture substrate. In some aspects, the population of hepatocytes and non-parenchymal cells may be disposed in a micropattern the population of hepatocytes and non-parenchymal cells may be disposed in a micropattern on a culture substrate. The micropattern may be a random or a predetermined two-dimensional pattern of multiple microdots, defined by a microdot diameter and an edge-to-edge spacing between each of any two neighboring microdots. For example, when the micropattern comprises a predetermined two-dimensional pattern of multiple microdots, each microdot may have a diameter of 10 μm to 1000 μm, and the edge-to-edge spacing between each microdot may be at least about 200 μm to about 1300 μm. The non-parenchymal cell population may occupy the inter-microdot space which is not occupied by the hepatocytes. The HSCs may occupy the inter-microdot space which is not occupied by the hepatocytes or may be dispersed throughout the co-culture.

In another aspect, the present disclosure provides a method of determining the potential for a negative interaction of a test compound with second compound, the method comprising: obtaining a co-culture of a population of hepatocytes, at least one non-parenchymal cell population, and a population of hepatic stellate cells in vitro; contacting the co-culture with the first and second test compounds; maintaining the co-culture for a time and under conditions sufficient to allow an effect of interaction of the first and second test compounds on the cells; measuring at least one at least one indicator of hepatocyte function in the hepatocytes to obtain a test measurement, or applying hepatocyte imaging technology (HIAT) to the hepatocytes to obtain a test image; and comparing the test measurement to a control measurement from the hepatocytes before contact with the first and second test compounds, or the test image to a control image before contact with the first and second test compounds, wherein a difference in the test measurement relative to the control measurement is indicative of the potential for a interaction of the test compound with the second compound. In another aspect, at least one indicator of HSC function is measured as well. The at least one indicator of cell function may be, for example, albumin production, urea production, ATP production, enzyme activity, lipid accumulation, glutathione production, liver gene expression, or liver protein expression in the hepatocytes and/or HSCs. The at least one enzyme activity can be selected for example from any inducible liver enzyme, including but not limited to a CYP enzyme such as CYP2C9 (luciferin-H), CYP3A4 (luciferin-IPA), a combination of CYP1A1, CYP1A2, CYP2B6 and CYP2D6 (luciferin ME-EGE), and any combination thereof. The hepatocytes may be primary human hepatocytes. The hepatocytes may be derived from any mammalian pluripotent stem cells. Hepatocytes may be obtained from one or more human donors suffering from a disorder of the liver. The disorder of the liver may be Type-2 diabetes, metabolic syndrome, non-alcoholic fatty liver disease (NAFLD), non-alcoholic steatohepatitis (NASH), and cardiovascular disease. At least one of the non-parenchymal cell populations may comprise stromal cells. Stromal cells may comprise fibroblasts, fibroblast-derived cells, macrophages, endothelial cells, stellate cells, pericytes, inflammatory cells, cholangiocytes and other types of stromal cells, and combinations thereof. For example, stromal cells may comprise 3T3-J2 murine embryonic fibroblasts. The at least one non-parenchymal cell population may be obtained from an individual not suffering from a disorder of the liver. The at least one non-parenchymal cell population may be obtained from an individual suffering from a disorder of the liver. The HSCs may be primary HSCs from one or more donors. The HSCs may be an immortalized HSC line. The HSCs may also be cancerous HSCs or animal-derived HSCs. The HSCs may be obtained from an individual not suffering from a disorder of the liver. The HSCs may be obtained from an individual suffering from a disorder of the liver. The HSCs may be in an activated state. The HSCs may be in a deactivated/quiescent state. The composition may further comprise a culture substrate. The culture substrate may be hard or soft. A hard culture substrate may comprise a glass surface, a polystyrene surface, or a silicon surface. A soft culture substrate may comprise an extracellular matrix, extracellular matrix gel, or hydrogel. The hard culture substrate may have a hardness of more than 5 kPa to about 60 GPa. The soft culture substrate may have a hardness of about 0.15 KPa to about 5 KPa. A biopolymer scaffold may be disposed on the culture substrate. In some aspects, the population of hepatocytes and non-parenchymal cells may be disposed in a micropattern on a culture substrate. In further aspects, the population of hepatocytes, non-parenchymal cells, and HSCs may be disposed in a micropattern on a culture substrate The micropattern may be a random or a predetermined two-dimensional pattern of multiple microdots, defined by a microdot diameter and an edge-to-edge spacing between each of any two neighboring microdots. For example, when the micropattern comprises a predetermined two-dimensional pattern of multiple microdots, each microdot may have a diameter of 10 μm to 1000 μm, and the edge-to-edge spacing between each microdot may be at least about 200 μm to about 1300 μm. The non-parenchymal cell population may occupy the inter-microdot space which is not occupied by the hepatocytes. The HSCs may occupy the inter-microdot space which is not occupied by the hepatocytes or may be dispersed throughout the co-culture. In one aspect, the population of HSCs may be cultured on a layer of material comprising at least one extracellular matrix protein that is disposed on the population of hepatocytes and at least one non-parenchymal cell population. In another aspect, the population of HSCs may be cultured on a substrate that is in fluid communication with the population of hepatocytes and at least one non-parenchymal cell population, and wherein the population of hepatic stellate cells is not in physical contact with the population of hepatocytes and at least one non-parenchymal cell population. The HSCs may be cultured on transwells. The transwells may be transferred to the co-culture of population of hepatocytes and at least one non-parenchymal cell population. The transwell may be absorbed with an extracellular matrix or extracellular matrix gels.

In another aspect, the present disclosure provides a method of determining the potential for a negative interaction of a test compound with second compound, the method comprising: co-culturing the population of hepatocytes with at least one non-parenchymal cell population and with conditioned medium obtained from a population of activated HSCs in vitro; contacting the co-culture with the first and second test compounds; maintaining the co-culture for a time and under conditions sufficient to allow an effect of interaction of the first and second test compounds on the cells; measuring at least one indicator of hepatocyte function to obtain a test measurement, or applying hepatocyte imaging technology (HIAT) to the hepatocytes to obtain a test image; and comparing the test measurement to a control measurement from the hepatocytes before contact with the first and second test compounds, or the test image to a control image before contact with the first and second test compounds, wherein a difference in the test measurement relative to the control measurement is indicative of the potential for a interaction of the test compound with the second compound. The at least one indicator of hepatic function may be, for example, albumin production, urea production, ATP production, enzyme activity, lipid accumulation, glutathione production, liver gene expression, or liver protein expression in the hepatocytes. The at least one enzyme activity can be selected for example from any inducible liver enzyme, including but not limited to a CYP enzyme such as CYP2C9 (luciferin-H), CYP3A4 (luciferin-IPA), a combination of CYP1A1, CYP1A2, CYP2B6 and CYP2D6 (luciferin ME-EGE), and any combination thereof. The hepatocytes may be primary human hepatocytes. The hepatocytes may be primary human hepatocytes. The hepatocytes may be derived from any mammalian pluripotent stem cells. Hepatocytes may be obtained from one or more human donors suffering from a disorder of the liver. The disorder of the liver may be Type-2 diabetes, metabolic syndrome, non-alcoholic fatty liver disease (NAFLD), non-alcoholic steatohepatitis (NASH), and cardiovascular disease. At least one of the non-parenchymal cell populations may comprise stromal cells. Stromal cells may comprise fibroblasts, fibroblast-derived cells, macrophages, endothelial cells, stellate cells, pericytes, inflammatory cells, cholangiocytes and other types of stromal cells, and combinations thereof. For example, stromal cells may comprise 3T3-J2 murine embryonic fibroblasts. The at least one non-parenchymal cell population may be obtained from an individual not suffering from a disorder of the liver. The at least one non-parenchymal cell population may be obtained from an individual suffering from a disorder of the liver. The conditioned medium is obtained from a population of activated hepatic stellate cells. The HSCs may be primary HSCs from one or more donors. The HSCs may be an immortalized HSC line. The HSCs may also be cancerous HSCs or animal-derived HSCs. The HSCs may be obtained from an individual not suffering from a disorder of the liver. The HSCs may be obtained from an individual suffering from a disorder of the liver. The HSCs may be cultured on a culture substrate. A hard culture substrate may comprise a glass surface, a polystyrene surface, or a silicon surface. A soft culture substrate may comprise an extracellular matrix, extracellular matrix gel, or hydrogel. The hard culture substrate may have a hardness of more than 5 kPa to about 60 GPa. The soft culture substrate may have a hardness of about 0.15 KPa to about 5 KPa. Similarly, the population of hepatocytes and at least one non-parenchymal cell population in co-culture may comprise a culture substrate. A biopolymer scaffold may be disposed on the culture substrate. In some aspects, the population of hepatocytes and non-parenchymal cells may be disposed in a micropattern the population of hepatocytes and non-parenchymal cells may be disposed in a micropattern on a culture substrate. The micropattern may be a random or a predetermined two-dimensional pattern of multiple microdots, defined by a microdot diameter and an edge-to-edge spacing between each of any two neighboring microdots. For example, when the micropattern comprises a predetermined two-dimensional pattern of multiple microdots, each microdot may have a diameter of 10 μm to 1000 μm, and the edge-to-edge spacing between each microdot may be at least about 200 μm to about 1300 μm. The non-parenchymal cell population may occupy the inter-microdot space which is not occupied by the hepatocytes. The HSCs may occupy the inter-microdot space which is not occupied by the hepatocytes or may be dispersed throughout the co-culture.

In another aspect, the present disclosure provides a method of determining whether a test compound alleviates hepatic dysfunctions cause by HSCs, the method comprising: obtaining a co-culture of a population of hepatocytes, at least one non-parenchymal cell population, and a population of hepatic stellate cells in vitro; contacting the co-culture with the test compound; maintaining the co-culture for a time and under conditions sufficient to allow an effect of test compound on the hepatocytes; and measuring at least one indicator of hepatic function in the hepatocytes to obtain a test measurement, or applying hepatocyte imaging technology (HIAT) to the hepatocytes to obtain a test image; and comparing the test measurement to a control measurement from the hepatocytes before contact with test compound, or the test image to a control image before contact with the compound, wherein a difference between the test and control is indicative of an effect of HSCs on hepatocyte function. In some aspects, the co-culture may be contacted with more than one test compound. The at least one indicator of hepatic function may be, for example, albumin production, urea production, ATP production, enzyme activity, lipid accumulation, glutathione production, liver gene expression, or liver protein expression in the hepatocytes. The at least one enzyme activity can be selected for example from any inducible liver enzyme, including but not limited to a CYP enzyme such as CYP2C9 (luciferin-H), CYP3A4 (luciferin-IPA), a combination of CYP1A1, CYP1A2, CYP2B6 and CYP2D6 (luciferin ME-EGE), and any combination thereof. The hepatocytes may be primary human hepatocytes. The hepatocytes may be primary human hepatocytes. The hepatocytes may be derived from any mammalian pluripotent stem cells. Hepatocytes may be obtained from one or more human donors suffering from a disorder of the liver. The disorder of the liver may be Type-2 diabetes, metabolic syndrome, non-alcoholic fatty liver disease (NAFLD), non-alcoholic steatohepatitis (NASH), and cardiovascular disease. At least one of the non-parenchymal cell populations may comprise stromal cells. Stromal cells may comprise fibroblasts, fibroblast-derived cells, macrophages, endothelial cells, stellate cells, pericytes, inflammatory cells, cholangiocytes and other types of stromal cells, and combinations thereof. For example, stromal cells may comprise 3T3-J2 murine embryonic fibroblasts. The at least one non-parenchymal cell population may be obtained from an individual not suffering from a disorder of the liver. The at least one non-parenchymal cell population may be obtained from an individual suffering from a disorder of the liver. The HSCs may be primary HSCs from one or more donors. The HSCs may be an immortalized HSC line. The HSCs may also be cancerous HSCs or animal-derived HSCs. The HSCs may be obtained from an individual not suffering from a disorder of the liver. The HSCs may be obtained from an individual suffering from a disorder of the liver. The HSCs may be in an activated state. The HSCs may be in a deactivated/quiescent state. The composition may further comprise a culture substrate. The culture substrate may be hard or soft. A hard culture substrate may comprise a glass surface, a polystyrene surface, or a silicon surface. A soft culture substrate may comprise an extracellular matrix, extracellular matrix gel, or hydrogel. The hard culture substrate may have a hardness of more than 5 kPa to about 60 GPa. The soft culture substrate may have a hardness of about 0.15 KPa to about 5 KPa. A biopolymer scaffold may be disposed on the culture substrate. In some aspects, the population of hepatocytes and non-parenchymal cells may be disposed in a micropattern on a culture substrate. In further aspects, the population of hepatocytes, non-parenchymal cells, and HSCs may be disposed in a micropattern on a culture substrate The micropattern may be a random or a predetermined two-dimensional pattern of multiple microdots, defined by a microdot diameter and an edge-to-edge spacing between each of any two neighboring microdots. For example, when the micropattern comprises a predetermined two-dimensional pattern of multiple microdots, each microdot may have a diameter of 10 μm to 1000 μm, and the edge-to-edge spacing between each microdot may be at least about 200 μm to about 1300 μm. The non-parenchymal cell population may occupy the inter-microdot space which is not occupied by the hepatocytes. The HSCs may occupy the inter-microdot space which is not occupied by the hepatocytes or may be dispersed throughout the co-culture. In one aspect, the population of HSCs may be cultured on a layer of material comprising at least one extracellular matrix protein that is disposed on the population of hepatocytes and at least one non-parenchymal cell population. In another aspect, the population of HSCs may be cultured on a substrate that is in fluid communication with the population of hepatocytes and at least one non-parenchymal cell population, and wherein the population of hepatic stellate cells is not in physical contact with the population of hepatocytes and at least one non-parenchymal cell population. The HSCs may be cultured on transwells. The transwells may be transferred to the co-culture of population of hepatocytes and at least one non-parenchymal cell population. The transwell may be absorbed with an extracellular matrix or extracellular matrix gels.

In another aspect, the present disclosure provides a method of determining whether a test compound alleviates hepatic dysfunctions cause by HSCs, the method comprising: co-culturing the population of hepatocytes with at least one non-parenchymal cell population and with conditioned medium obtained from a population of activated HSCs in vitro; contacting the co-culture with the test compound; maintaining the co-culture for a time and under conditions sufficient to allow an effect of test compound on the hepatocytes; and measuring at least one indicator of hepatic function in the hepatocytes to obtain a test measurement, or applying hepatocyte imaging technology (HIAT) to the hepatocytes to obtain a test image; and comparing the test measurement to a control measurement from the hepatocytes before contact with test compound, or the test image to a control image before contact with the compound, wherein a difference between the test and control is indicative of an effect of HSCs on hepatocyte function. In some aspects, the co-culture may be contacted with more than one test compound. The at least one indicator of cell function may be, for example, albumin production, urea production, ATP production, enzyme activity, lipid accumulation, glutathione production, liver gene expression, or liver protein expression in the hepatocytes. The at least one enzyme activity can be selected for example from any inducible liver enzyme, including but not limited to a CYP enzyme such as CYP2C9 (luciferin-H), CYP3A4 (luciferin-IPA), a combination of CYP1A1, CYP1A2, CYP2B6 and CYP2D6 (luciferin ME-EGE), and any combination thereof. The hepatocytes may be primary human hepatocytes. The hepatocytes may be primary human hepatocytes. The hepatocytes may be primary human hepatocytes. The hepatocytes may be derived from any mammalian pluripotent stem cells. Hepatocytes may be obtained from one or more human donors suffering from a disorder of the liver. The disorder of the liver may be Type-2 diabetes, metabolic syndrome, non-alcoholic fatty liver disease (NAFLD), non-alcoholic steatohepatitis (NASH), and cardiovascular disease. At least one of the non-parenchymal cell populations may comprise stromal cells. Stromal cells may comprise fibroblasts, fibroblast-derived cells, macrophages, endothelial cells, stellate cells, pericytes, inflammatory cells, cholangiocytes and other types of stromal cells, and combinations thereof. For example, stromal cells may comprise 3T3-J2 murine embryonic fibroblasts. The at least one non-parenchymal cell population may be obtained from an individual not suffering from a disorder of the liver. The at least one non-parenchymal cell population may be obtained from an individual suffering from a disorder of the liver. The conditioned medium is obtained from a population of activated hepatic stellate cells. The HSCs may be primary HSCs from one or more donors. The HSCs may be an immortalized HSC line. The HSCs may also be cancerous HSCs or animal-derived HSCs. The HSCs may be obtained from an individual not suffering from a disorder of the liver. The HSCs may be obtained from an individual suffering from a disorder of the liver. The HSCs may be cultured on a culture substrate. A hard culture substrate may comprise a glass surface, a polystyrene surface, or a silicon surface. A soft culture substrate may comprise an extracellular matrix, extracellular matrix gel, or hydrogel. The hard culture substrate may have a hardness of more than 5 kPa to about 60 GPa. The soft culture substrate may have a hardness of about 0.15 KPa to about 5 KPa. Similarly, the population of hepatocytes and at least one non-parenchymal cell population in co-culture may comprise a culture substrate. A biopolymer scaffold may be disposed on the culture substrate. In some aspects, the population of hepatocytes and non-parenchymal cells may be disposed in a micropattern the population of hepatocytes and non-parenchymal cells may be disposed in a micropattern on a culture substrate. The micropattern may be a random or a predetermined two-dimensional pattern of multiple microdots, defined by a microdot diameter and an edge-to-edge spacing between each of any two neighboring microdots. For example, when the micropattern comprises a predetermined two-dimensional pattern of multiple microdots, each microdot may have a diameter of 10 μm to 1000 μm, and the edge-to-edge spacing between each microdot may be at least about 200 μm to about 1300 μm. The non-parenchymal cell population may occupy the inter-microdot space which is not occupied by the hepatocytes. The HSCs may occupy the inter-microdot space which is not occupied by the hepatocytes or may be dispersed throughout the co-culture.

### BRIEF DESCRIPTION OF THE FIGURES

The application file contains at least one drawing executed in color. Copies of this patent application publication with color drawing(s) will be provided by the Office upon request and payment of the necessary fee.

FIG. 1 depicts the fabrication of fibrotic and healthy contact co-culture models. FIG. 1A: Extracellular matrix (e.g., collagen)-coated tissue culture plastic is first micropatterned using soft lithography or other techniques. Hepatocytes are then seeded onto patterned matrix. Supportive murine embryonic fibroblasts with or without hepatic HSCs are seeded onto micropatterned hepatocyte cultures the next day to establish healthy and fibrotic liver models. FIG. 1B: HSC can be cultured with hepatocyte/fibroblast micropatterned co-cultures in an activated state on hard/stiff plastic (which spontaneously activates HSCs) to create a fibrosis model. FIG. 1C: The hepatocyte-fibroblast micropatterned co-culture can be overlaid with a gel (e.g., made of collagen and/or Matrigel™) of varying compliances and HSCs cultured on top of the gel to create a liver model in which the HSCs are not activated to the same degree as on plastic, thereby mimicking a liver closer to a healthy state.

FIG. 2 depicts the fabrication of fibrotic and healthy paracrine co-culture models. Transwells are adsorbed with extracellular matrix such as collagen (stiff) or extracellular matrix gels (soft with varying compliances). HSCs are then seeded onto adsorbed matrix or matrix gels and allowed to attach. Micropatterned co-cultures containing hepatocytes and murine embryonic fibroblasts are established. Transwells containing HSCs on various substrates are then transferred to micropatterned co-cultures to make activated (stiff) or less activated/quiescent (soft) co-cultures. In these culture models, in contrast to those in FIG. 1, the hepatocytes and HSCs are not contacting each other but communicating with secreted molecules (paracrine signaling).

FIG. 3 depicts primary HSCs only support long-term hepatocyte functions at physiologically relevant concentrations. FIG. 3A-FIG. 3E: Experimental setup for long term hepatocyte culture. FIG. 3A: Type I rat tail collagen is coated on polystyrene and plasma etched into microdomains using an elastomeric stamp. FIG. 3B: Cryopresevered primary human hepatocytes selectively adhere to these domains on day 0 and the next day 3T3-J2 fibroblasts (MPBCs) (FIG. 3C) and/or primary HSCs (MPBC-HSC and MPTC) (FIG. 3D, FIG. 3E) are seeded into these cultures where they surround hepatocyte “islands.” Phase contrast images of each culture setup from day 0 (FIG. 3B) and day 7 (FIG. 3C-FIG. 3E) are shown next to the schematic. MPBC (FIG. 3F) and MPBC-HSC (FIG. 3G) albumin, urea (top) and CYP450 3A4 activity (bottom) over 2 weeks of culture are shown. MPTC albumin (FIG. 3H, top) and urea (FIG. 3H, bottom) output for the various concentrations of HSCs are shown over 2 weeks of culture. Error bars represent standard deviation (“SD”). Results are from a representative experiment.

FIG. 4 depicts that HSC-hepatocyte-fibroblast micropatterned tri-cultures maintain basic hepatocyte functions while acquiring a steatotic profile. Cultures are created as shown in FIG. 1B. FIG. 4A, FIG. 4B: Albumin and urea production over time in control and experimental co-cultures from days 4-14. Note that “0 HSCs” indicates a hepatocyte-fibroblast micropatterned co-culture without HSCs, while the other conditions indicate micropatterned co-cultures containing the noted numbers of HSCs with the fibroblasts. Error bars represent SD. FIG. 4C-FIG. 4F: Hepatocyte morphology in control and experimental conditions at day 14 of culture. FIG. 4C corresponds to the “0 HSCs” condition, while pictures FIG. 4D, FIG. 4E, and FIG. 4F correspond to “1.25K HSCs”, “2.5K HSCs” and “5K HSCs” conditions, respectively. FIG. 4C is a micropatterned co-culture without HSCs while FIG. 4D, FIG. 4E, and FIG. 4F pictures contain HSCs at increasing densities (1.25K, 2.5K or 5K HSCs mixed in with fibroblasts in hepatocyte-fibroblast co-cultures, respectively). Notice the lipid accumulation in conditions where HSCs are present. This lipid accumulation also occurs in pure hepatocyte cultures cultured on gels to sustain phenotype.

FIG. 5 depicts activated stellate cells cause down regulation of drug metabolism pathways/enzyme activity in primary human hepatocytes. At low densities (5 K) in MPTCs (FIG. 5A), PKH dyed HSCs (FIG. 5B) (green, white arrows) can be seen surrounding (left image) and on top of (right image) hepatocyte islands (dotted line shows edge) (magenta). FIG. 5C: CYP 3A4 enzyme activity of MPTCs with a range of initial HSC seeding densities (0-5 K) at 2 weeks of culture (*0 HSC is the same data from MPBC in FIG. 3F for comparison). FIG. 5D: CYP 2A6 enzyme activity of MPTCs with a range of initial HSC seeding densities (0-5 K) after 2 weeks of culture. FIG. 5E: Gene expression of HSC activation markers, LOX and COL1A1, after 2 weeks of culture in MPTCs normalized to HSC RNA isolated on the day of seeding. HPRT was the house keeping gene. FIG. 5F: CYP 3A4, NR112, NR113, HNF4a and NRF2 gene expression in MPTCs cultured with 2.5 K HSCs at 2 weeks, normalized to HSC free MPBCs. GAPDH was the house keeping gene. Scale bar represents 80 μM. Error bars represent SD. *, **, ***, **** represent p values of 0.05, 0.01, 0.001, and 0.0001, respectively. One way ANOVAs were carried out using well replicates and comparing values to their respective control for (FIG. 5C-FIG. 5E) and students t-tests, between 0 HSC controls and HSC containing cultures, were used to identify significance in (FIG. 5F).

FIG. 6 depicts HSC number dependent decrease in hepatic CYP enzyme activity and their effects on liver transcription factors. Cultures were created as shown in FIG. 1B but with varying numbers of HSCs. Note that “0 HSCs” indicates a hepatocyte-fibroblast micropatterned co-culture without HSCs, while the other conditions indicate micropatterned co-cultures containing the noted numbers of HSCs with the fibroblasts. FIG. 6A: CYP 2A6 activity, assessed by coumarin conversion to 7-hydroxycoumarin (7-HC), at 2 weeks of culture in the various models. FIG. 6B: CYP3A4 activity, assessed by luciferin-IPA Promega PGIo kit, at 1 and 2 weeks of culture. Error bars represent SD. FIG. 6C: Hepatocyte nuclear factor 4 alpha (HNF4a) and hepatocyte nuclear factor 6 (ONECUT1) gene expression at 2 weeks of culture. FIG. 6D: Aryl hydrocarbon receptor (AHR) and constitutive androstane receptor (CAR) gene expression at 2 weeks of culture. Fold gene expression is shown relative to “0 HSCs” control co-cultures and normalized to the house keeping gene Glyceraldehyde 3-phosphate dehydrogenase (GAPDH).

FIG. 7 depicts activated HSC conditioned medium causes down regulation of pure hepatocyte culture CYP 3A4 activity. FIG. 7A: Pure hepatocyte cultures were treated with either normal media or HSC conditioned media for 6 days and then assessed for CYP 3A4 activity. FIG. 7B: Phase contrast image of pure hepatocytes after 6 days of culture. Error bars represent SD and * represents a p-value of 0.05, using a student's t-test. Scale bar represents 80 μM.

FIG. 8 depicts density dependent activation of HSCs in tri-cultures. Cultures were created as shown in FIG. 1B and lysed for RNA on day 14. Note that “0 HSCs” indicates a hepatocyte-fibroblast micropatterned co-culture without HSCs, while the other conditions indicate micropatterned co-cultures containing the noted numbers of HSCs with the fibroblasts. HSC activation markers include alpha smooth muscle actin (ASMA2), lysyl oxidase (LOX) and collagen type I (COL1A1). Fold gene expression is shown relative to “fresh HSCs” (before co-culturing with hepatocyte-fibroblasts) and normalized to the house keeping gene hypoxanthine-guanine phosphoribosyltransferase (HPRT).

FIG. 9 depicts down regulation of drug metabolism genes with 2 different hepatocyte donors and stellate cell donors and ECM accumulation in MPTCs. FIG. 9A: Gene expression of CYP 3A4, NR1I2, NR1I3, HNF4a and NRF2 in MPTCS with human hepatocyte donor 1 and 2 using stellate cell donor 1 ((left) also in FIG. 5) and stellate cell donor 2 (right). GAPDH was the house keeping gene. FIG. 9B: Pico sirius red staining of ECM accumulation in MPTCs and 0 HSC MPBC control with increasing HSC number at 2 weeks of culture. Scale bar represents 1 mm. Error bars represent SD. *, **, ***, **** represent p values of 0.05, 0.01, 0.001, and 0.0001, respectively. Students t-tests, between 0 HSC controls and HSC containing cultures, were used to identify significance.

FIG. 10 depicts activated HSCs decrease bile/drug transporter function and gene expression. FIG. 10A, FIG. 10B: Day 13 live-cell images of micropatterned hepatocyte island CDCFDA secretions (green-MRP2) and Hoechst 33342 nuclear stain (blue) with 2.5 K HSCs (FIG. 10B) or no (FIG. 10A) HSCs added on day 1. Scale bars represent 400 μM. FIG. 10C: Gene expression of basolateral uptake transporters, SLCO1B1 (OATP1B1), SLC01B3 (OATP1B3), and SLC10A1 (NTCP), basolateral export transporters, ABCC1 (MRP1), ABCC3 (MRP3), and ABCC4 (MRP4), and canlicular export transporters, ABCC2 (MRP2), and ABCB11 (BSEP), in MPTCs normalized to 0 HSC control. RNA was collected after 2 weeks of culture and the housekeeping gene used was GAPDH. Error bars represent SD. *, **, ***, **** represent p values of 0.05, 0.01, 0.001, and 0.0001, respectively. Students t-tests, between 0 HSC controls and HSC containing cultures, were used to identify significance.

FIG. 11 depicts activated HSCs decrease bile/drug transporter function and gene expression in 2 hepatocyte donors. FIG. 11A, FIG. 11B: Day 13 live-cell images of micropatterned hepatocyte island CDCFDA secretions (green-MRP2) and Hoechst 33342 nuclear stain (blue) with 2.5 K HSCs (donor 2) (FIG. 11B) or no (FIG. 11A) HSCs added on day 1 (*same image as FIG. 10A for comparison). Scale bars represent 400 μM. FIG. 11C: Gene expression of basolateral uptake transporters, SLCO1B1 (OATP1B1), SLC01B3 (OATP1B3), and SLC10A1 (NTCP), basolateral export transporters, ABCC1 (MRP1), ABCC3 (MRP3), and ABCC4 (MRP4), and canlicular export transporters, ABCC2 (MRP2), and ABCB11 (BSEP), in MPTCs, using hepatocyte donors 1 and 2 with HSC donor 1, normalized to 0 HSC control. FIG. 11D: Same as FIG. 11C except HSC donor 2 was used and SLCO1B1, SLCO1B3, ABCC1, and ABCC4 were not assessed in hepatocyte donor 2. RNA was collected after 2 weeks of culture and the housekeeping gene was GAPDH. Error bars represent SD. *, **, ***, **** represent p values of 0.05, 0.01, 0.001, and 0.0001, respectively. Students t-tests, between 0 HSC controls and HSC containing cultures, were used to identify significance.

FIG. 12 depicts HSCs cause hepatocyte neutral lipid accumulation in a time and density dependent manner. FIG. 12A: Phase contrast images of MPBCs (top) and MPTCs (bottom) at day 9 (left), day 11 (center) and day 13 (right) of culture. FIG. 12B: Representative Nile red, neutral lipid, stained hepatocyte islands of MPBCs (0 HSCs) and MPTCs with 1.25 K, 2.5 K and 5K HSCs after 2 weeks of culture. Circles outline hepatocyte islands. Scale bars represent 400 μM.

FIG. 13 depicts activated HSCs alter lipid metabolism and cellular stress response gene expression. Cultures were created as shown in FIG. 13B. Note that “0 HSCs” indicates a hepatocyte-fibroblast micropatterned co-culture without HSCs, while the other conditions indicate micropatterned co-cultures containing the noted numbers of HSCs with the fibroblasts. FIG. 13A: CYP 7A1, fatty acid synthase (FASN), carbohydrate response element binding protein (ChREBP), ELOVL fatty acid elongase 2 (ELOVL2) and CD-36 gene expression at 2 weeks of culture. FIG. 13B: Nuclear factor (erythroid-derived 2)-like 2 (NFE2L2) and C-FOS (FOS) gene expression at 2 weeks of culture. FIG. 13C: Glucose 6 phosphatase catalytic subunit (G6PC) and phosphoenolpyruvate carboxykinase 1 (PCK1) gene expression at 2 weeks of culture. Fold gene expression is shown relative to “0 HSCs” control co-cultures and normalized to the house keeping gene glyceraldehyde 3-phosphate dehydrogenase (GAPDH).

FIG. 14 depicts activated hepatic stellate cell secreted interleukin 6 (IL-6) decreases hepatocyte CYP 3A4 activity. FIG. 14A: Pure HSCs conditioned medium was collected and placed on MPBCs for 2 days at a time. FIG. 14B: MPBCs were continuously conditioned with pure stellate cell medium (5 K density) from day 3-13 of culture and CYP 3A4 activity was assessed on day 5, 7 and 13. FIG. 14C: Pure HSC (5 K density) IL-6 secretions over time. FIG. 14D: Effect of anti-IL-6 or anti-IgG neutralizing antibodies on 3A4 activity of MPBCs conditioned with HSC media over time (*Non-conditioned treatment is the same data from b for comparison). Error bars represent SD. *, **, ***, **** represent p values of 0.05, 0.01, 0.001, and 0.0001, respectively. Students t-tests (FIG. 14B) and one way ANOVAs (FIG. 14D) were carried out using well replicates and comparing values to their respective control to identify significance.

FIG. 15 depicts MPTCs have increased IL-6 and hepatic stellate cell (donor 2) secreted interleukin 6 (IL-6) decreases hepatocyte CYP 3A4 activity. FIG. 15A, FIG. 15B: MPTCs with 2.5 K HSCs (donor 1 (FIG. 5A), donor 2 (FIG. 15B)) were assessed for CYP 3A4 activity (left) and IL-6 secretion (right) at day 7 and 13 of culture. (FIG. 15C, left) MPBCs were continuously conditioned with pure stellate cell medium (donor 2 (5 K density)) from day 3-13 of culture and CYP 3A4 activity was assessed on day 5, 7 and 13. (FIG. 15C, right) Pure HSC (donor 2 (5 K density) IL-6 secretions over time. FIG. 15D: Effect of anti-IL-6 or anti-IgG neutralizing antibodies on 3A4 activity of MPBCs conditioned with HSC media over time (*Non-conditioned treatment is the same data from c for comparison). Error bars represent SD. *, **, ***, **** represent p values of 0.05, 0.01, 0.001, and 0.0001, respectively. Students t-tests (FIG. 15A-FIG. 15C) and one way ANOVAs (FIG. 15D) were carried out using well replicates and comparing values to their respective control to identify significance.

FIG. 16 depicts stiffness mediated hepatic stellate cell activation alters CYP 3A4 and IL-6 secretion. HSCs were cultured on type I collagen gels or collagen coated plastic/glass (FIG. 16B) and their medium was collected every 2 days and placed on MPBCs. FIG. 16A: MPBC CYP 3A4 activity over time with conditioning from HSCs on gels or plastic/glass compared to a non-conditioned MPBC control (*Non-conditioned MPBC in this panel is the same data from FIG. 14A and FIG. 14D for comparison). FIG. 16B: Representative phase contrast and immunofluorescent anti-YAP labeling of HSCs cultured on collagen gels (left), and plastic/glass (right and respective nuclear labeling with DAPI below). FIG. 16C: Ratio of cytoplasmic to nuclear (C:N) labeling of anti-YAP in HSCs cultured as in FIG. 6B. n=52 cells for glass, and n=52 cells for soft gels. FIG. 16D: IL-6 secretions from pure HSCs seeded on either gels or plastic/glass (*same data shown in FIG. 14C for comparison) on day 5 of culture. Error bars represent SD. *,**,***,**** represent p values of 0.05, 0.01, 0.001, and 0.0001, respectively. One way ANOVAs (FIG. 6A) and students t tests (FIG. 16C, FIG. 16D) were carried out using well replicates and comparing values to their respective control to identify significance.

FIG. 17 depicts substrate mediated modulation of HSC-hepatocyte interaction. HSCs were seeded onto hard/stiff collagen-coated plastic, which induces their activation, or soft gels (3 mg/ml type I rat tail collagen) and cultured for 1 week. 3 week old hepatocyte-fibroblast micropatterned co-cultures were then exposed to conditioned medium from HSCs on the 2 different substrates. HSC morphology on (FIG. 17A) collagen coated plastic and (FIG. 17B) 3 mg/ml collagen gel at 2 weeks of culture. Micropatterned co-culture morphology after being conditioned for ˜1 week with medium from HSCs cultured on (FIG. 17C) collagen coated plastic and (FIG. 17D) 3 mg/ml collagen gel. (FIG. 17E) Micropatterned co-culture CYP3A4 activity after being conditioned for ˜1 week with medium from HSCs cultured on collagen coated plastic (hard) and 3 mg/ml collagen gel (soft). Error bars represent SD.

FIG. 18 depicts stiffness mediated activation of HSCs causes down regulation of CYP 3A4 in MPBCs and increases HSC IL-6 production. HSC donor 2 (FIG. 18A) or 3 (FIG. 18B) cells were seeded on either type I collagen gels or coated plastic/glass and their IL-6 secretions (right) were measured on day 5 of culture. Additionally, the conditioned medium from these pure HSC cultures was added to MPBCs from day 3-13, and CYP 3A4 activity (left) was assessed. Error bars represent SD. *,**,***,**** represent p values of 0.05, 0.01, 0.001, and 0.0001, respectively. Students t-tests were carried out using well replicates and comparing values to their respective control to identify significance.

FIG. 19 depicts TGFb treatment and TGFb receptor inhibition effects on hepatocyte-fibroblast micropatterned co-cultures and hepatocyte-fibroblast micropatterned co-cultures conditioned with HSC culture medium. FIG. 19A: Hepatocyte-fibroblast micropatterned co-cultures were treated with increasing amounts of TGFb growth factor and a dose dependent decrease in 3A4 activity was observed after 10 days of treatment. FIG. 19B: Hepatocyte-fibroblast micropatterned co-cultures were pretreated with the TGFb receptor inhibitor SB431542 at increasing doses for 2 hours, then incubated with HSC conditioned medium containing the inhibitor for 10 days, and then 3A4 activity was assessed. A dose dependent increase in 3A4 activity was observed with the addition of the inhibitor, which suggests that inhibiting TGFb signaling in hepatocytes could ameliorate the negative effects of activated HSCs on hepatocytes. Error bars represent SD.

FIG. 20 depicts insulin and retinol modulate HSC IL-6 production, and subsequent CYP 3A4 activity in conditioned MPBCs. FIG. 20A: Day 5 IL-6 production from HSC cultures on collagen gels or coated plastic/glass treated with low (500 pM) or high (1 μM) insulin or retinol plus high insulin. FIG. 20B: The effects of placing the HSC conditioned medium from FIG. 20A on MPBC CYP 3A4 activity after 2 days of treatment with conditioned medium (day 7 of culture). Error bars represent SD. *,**,***,**** represent p values of 0.05, 0.01, 0.001, and 0.0001, respectively. One-way ANOVAs were carried out using well replicates and comparing values to their respective control to identify significance.

FIG. 21 depicts compliance and hormonal stimuli differentially alter HSC effects on hepatocyte CYP3A4 activity. Representative images of donor 1 (D1), 2 (D2) and 3 (D3) HSCs on day 6 of culture on collagen gels (FIG. 21A) or coated plastic/glass (FIG. 21B) seeded at a density of 5K per gel (250 uL at 2 mg/ml in 24 well format). FIG. 21C: IL-6 secretion of HSC D1 (left axis), HSC D2 (right axis) and HSC D3 (right axis) on soft collagen gels treated with 500 pM insulin, 1 μM insulin (normal hepatocyte maintenance media), and 1 μM insulin plus 2 μM all-trans retinol (retinol). FIG. 21E: 3A4 activity of MPBCs treated (2 days) with HSC conditioned media in FIG. 21C on day 7 of culture and normalized to 3A4 activity of MPBCs treated with HSCs cultured in 1 μM insulin on glass/plastic (FIG. 21F). Prior to this analysis, MPBCs were conditioned with the same media from day 3 to day 5. FIG. 21D, FIG. 21F: Same as (FIG. 7C, FIG. 7E), except HSCs were cultured on glass/plastic instead of gels. Error bars represent SD. *,**,***,**** represent p values of 0.05, 0.01, 0.001, and 0.0001, respectively. One way ANOVAs were carried out using well replicates and comparing values to their respective control.

FIG. 22 depicts paracrine factors mediate HSC-hepatocyte interactions. Hepatocyte-fibroblast micropatterned co-cultures, mono-cultures of 3T3-J2 fibroblasts, and co-cultures of HSCs and 3T3-J2 fibroblasts were established simultaneously. Medium from 3T3-J2 mono-cultures or HSC-fibroblast co-cultures was conditioned for 48 hours, sterile filtered (0.2 μm), and then placed on hepatocyte-fibroblast micropatterned co-cultures for an additional 48 hours to assess the effects of paracrine signaling on hepatocyte functions. FIG. 22A, FIG. 22B: Albumin and urea production in hepatocyte-fibroblast micropatterned co-cultures conditioned with either 3T3-J2 mono-cultures or HSC-fibroblast co-cultures conditioned medium. FIG. 22C, FIG. 22D: CYP2A6 and 3A4 activity in hepatocyte-fibroblast micropatterned co-cultures conditioned with either 3T3-J2 mono-cultures or HSC-fibroblast co-cultures conditioned medium. Note the similar 3A4 activity in 3T3-J2 mono-cultures and non-conditioned medium controls. This decrease in hepatocyte 3A4 activity also occurs in pure hepatocyte cultures cultured on gels to sustain phenotype (not shown). Error bars represent SD.

FIG. 23 depicts Valproic acid (VPA) helps reduce the negative effects of HSCs on hepatocytes. Cultures were created as shown in FIG. 1B and conditioned with VPA at 2.5 mM for 2 weeks. “J2” condition refers to hepatocyte-fibroblast micropatterned co-cultures while “J2+4.5 HSC” refers to hepatocyte-fibroblast-HSC micropatterned co-cultures with the indicated numbers of HSCs in thousands of cell numbers. FIG. 23A: CYP3A4 activity in cultures at 1 and 2 weeks with or without addition of VPA. Error bars represent SD. FIG. 23B: Morphology in cultures at 2 weeks with or without addition of VPA.

FIG. 24 depicts GKT137831 and obeticholic acid alleviates the detrimental effects of activated HSCs on hepatocytes. FIG. 24A: CYP 3A4 activity of MPTCs treated with either DMSO, GKT137831, or obeticholic acid (OCA) at 4 or 10 days of treatment normalized to 0 HSC, MPBC controls (dashed line represents MPBC CYP 3A4 level). FIG. 24B: NR1I2 gene expression in the same cultures at day 12 of treatment, normalized to a DMSO treated MPTC control. FIG. 24C: Functional bile transporter day dye, CDCFDA, fluorescence (green) in treated cultures after 12 days of treatment. FIG. 24D: ABCC2 gene expression in the same cultures at day 12 of treatment, normalized to a DMSO treated MPTC control. FIG. 24E: IL-6 in the supernatants of MPTC cultures treated with compounds for 6 days. FIG. 24F: Nile red staining of MPTCs and MPBCs treated with compounds or DMSO for 12 days. Scale bars represent 80 μM. Error bars represent SD. *,**,***,**** represent p values of 0.05, 0.01, 0.001, and 0.0001, respectively. One way ANOVAs were carried out using well replicates and comparing values to their respective control to identify significance.

FIG. 25 depicts GKT137831 and obeticholic acid administration alleviates the detrimental effects of activated HSCs on hepatocytes. FIG. 25A: 3A4 activity of MPTCs treated with either DMSO, GKT137831 (GKT), or obeticholic acid (OCA) at 4 or 10 days of treatment. FIG. 25B: NR1I2 gene expression in the same cultures at day 12 of treatment, normalized to a DMSO treated MPTC control. FIG. 25C: ABCC2 gene expression in the same cultures at day 12 of treatment, normalized to a DMSO treated MPTC control. FIG. 25D: IL-6 in the supernatants of MPTC cultures treated with compounds for 6 days. Error bars represent SD. *,**,***,**** represent p values of 0.05, 0.01, 0.001, and 0.0001, respectively. One way ANOVAs were carried out using well replicates and comparing values to their respective control to identify significance.

FIG. 26 depicts a drug screen using MPTC, which identified GKT and OCA as potential positive hits. FIGS. 26A, B: Effects of various compounds at different concentrations on MPTC (2.5 K HSC density) CYP 3A4 activity with hepatocyte donor 2 and HSC donor 1 and 3 at day 4 (FIG. 26A) and 10 (FIG. 26B) of treatment. Effects of various compounds at different concentrations on MPTC (2.5 K HSC density) CYP 3A4 activity with hepatocyte donor 1 and HSC donor 1 (* same data used in FIG. 24a) and 2 (* same data used in FIG. 25A) at day 4 (FIG. 26C) and 10 (FIG. 26D) of treatment. Error bars represent SD.

FIG. 27 depicts toxicity of compounds used in drug screen and effects of other compounds on Nile red staining and transporter function. Urea (FIG. 27A) and albumin (FIG. 27B), liver toxicity markers, production in MPTCs at 4 and 10 days of treatment with various compounds. Error bars represent SD. Checkered bar represents a condition, retinol, where values fell more than 50% below the DMSO control. (FIGS. 27C, D) Nile red (FIG. 27C) staining and transporter function assessed with CDCFDA stain (FIG. 27D) of DMSO, retinol, 0.1 μM OCA, and 100 μM resveratrol treated MPTCs at 12 days of treatment. Error bars represent SD. Scale bars represent 400 μM (FIG. 27C) and 80 μM (FIG. 27D).

### DETAILED DESCRIPTION

The present disclosure describes compositions and methods based in part on the surprising finding that the presence of activated HSCs in co-culture with human hepatocytes and non-parenchymal cells led to changes in hepatocyte lipid profile and negatively affect activities of hepatocyte drug metabolism enzymes such as cytochrome P450s. The presence of activated HSCs in co-couture also surprisingly affected other hepatic genes such as those associated with lipid metabolism and cellular stress response.

Unless otherwise required by context, singular terms as used herein and in the claims shall include pluralities and plural terms shall include the singular. For example, reference to “a cellular island” includes a plurality of such cellular islands and reference to “the cell” includes reference to one or more cells known to those skilled in the art, and so forth.

The use of “or” means “and/or” unless stated otherwise. Furthermore, the use of the term “including,” as well as other forms, such as “includes” and “included,” is not limiting. Also, terms such as “element” or “component” encompass both elements and components comprising one unit and elements and components that comprise more than one subunit unless specifically stated otherwise.

Described herein are several definitions. Such definitions are meant to encompass grammatical equivalents.

The term “co-culture” means the growth of more than one distinct cell type in a combined culture. Co-cultures of the present disclosure can include two or more distinct cell types. In some aspects, three or more distinct cell types are included in a co-culture. Co-cultures include, but are not limited to, cultures where two or more cell types are contained in the same container. This includes configurations where one or more of the cell types are contained within a transwell or similar device that is in contact with a container housing one or more cell types.

The term “donor” includes human and other mammalian subjects from which cells such as stem cells and/or primary hepatocytes may be obtained.

The term “patient” includes human and other mammalian subjects that receive either prophylactic or therapeutic treatment.

The term “population,” when referring to a “cell population,” “population of . . . cells,” and the like, refers to a group of cells of a distinct cell type. The population of cells may contain cells of the same distinct cell type obtained from one or more donors. In other aspects, the population of cells may contain cells of the same distinct type obtained from one or more cell lines.

The term “subject” refers to an animal, including but not limited to a mammal including a human and a non-human primate (for example, a monkey or great ape), a cow, a pig, a cat, a dog, a rat, a mouse, a horse, a goat, a rabbit, a sheep, a hamster, a guinea pig). Preferably, the subject is a human.

### A. Co-Cultures of Hepatocytes, Supportive Stromal Cells, and Hepatic Stellate Cells

The present disclosure encompasses a composition comprising a population of hepatocytes, at least one non-parenchymal cell population, and HSCs in co-culture. The present disclosure also encompasses a composition comprising a population of hepatocytes and at least one non-parenchymal cell population which is exposed to conditioned medium obtained from an HSC culture. The co-cultures as described herein provide a useful in vitro liver model and thus also provide a unique platform for the development and toxicology screening of therapeutic agents, including high-throughput screening of drug candidates for efficacy and toxicity. The co-cultures described herein include, but are not limited to, random co-cultures and micropatterned co-cultures (“MPCC”). The parenchymal cell population, at least one non-parenchymal cell population, and HSCs maybe in contact co-culture models or paracrine co-culture models.

The hepatocytes are mammalian, including human hepatocytes. The human hepatocytes can be, for example, primary human hepatocytes (“PHHs”), human hepatocytes derived from any mammalian pluripotent stem cells, and an immortalized human hepatocyte cell line. The human hepatocytes may be obtained from a normal mammalian donor or a mammalian donor suffering from a disorder of the liver. Disorders of the liver include, but are not limited to, metabolic disorders such as Type 2 diabetes, metabolic syndrome, non-alcoholic fatty liver disease (“NAFLD”), non-alcoholic steatohepatitis (“NASH”), and cardiovascular disease. The present disclosure encompasses a population of hepatocytes obtained from one or more human donors. A non-limiting, example co-culture is one which includes hepatocytes obtained from one or more human donors suffering from NAFLD or NASH. Another non-limiting, example co-culture is one which includes hepatocytes obtained from one or more normal human donors. Additionally, the human hepatocytes may be derived from any pluripotent stem cells, for example human induced pluripotent stem cells (“iPSC's”), embryonic stem cells (“ESC's”), hepatic resident stem cells (oval cells), and the like. A non-limiting, example co-culture is one which includes hepatocytes derived from human induced pluripotent stem cells such as iCell® Hepatocytes (“iHep” or “iHeps”) available from Cellular Dynamics International of Madison, Wis. The pluripotent stem cell may be from a normal or a diseased donor.

The non-parenchymal cells are may be human or non-human. At least one of the non-parenchymal cell populations may comprise stromal cells, such as but not limited to: fibroblasts, fibroblast-derived cells, macrophages, endothelial cells, pericytes, inflammatory cells, cholangiocytes, and other types of stromal cells, and combinations thereof. Fibroblasts may be for example mammalian fibroblasts, such as murine embryonic fibroblasts. Non-limiting, exemplary fibroblasts are 3T3-J2 murine embryonic fibroblasts. Human non-parenchymal cells from normal and diseased patients can also be used. It is contemplated that other non-parenchymal cells, both liver and non-liver, and non-parenchymal cells specifically implicated in a disease can be used to provide an in vitro model for drug testing new drugs to treat the disease.

Additionally, the present disclosure contemplates that hepatocytes obtained from an individual donor can be cultured with disease causing non-parenchymal cells or HSCs obtained from the same individual donor. Non-parenchymal cells such as endothelial cells can also be derived from iPSCs (e.g., iEndothelial, iMacrophage and the like). Non-parenchymal cells such as endothelial cells can also be derived for example from the same individual donor via an iPSC intermediary. HSCs can be derived from pluripotent stem cells. HSCs can also be derived, for example, from the same individual donor. The resulting co-culture can be used to screen optimal drug formulations to treat the disease (e.g., fibrosis) and resulting hepatic dysfunction in the individual donor. Hepatocyte colonies can be surrounded with different stromal cell populations to create liver models enabling controlled investigations of specific heterotypic interactions on hepatic functionality and maturation. For example, a combination of human mesenchymal stem cells and endothelial cells may also provide a supportive stromal environment. Additionally, small molecules for hepatocyte maturation can also be applied in the co-cultures described herein model. Small molecule hepatocyte maturation factors can be any from among those described in the literature and as known to those of skill in the art, including for example any of the small molecules described in Shan et al., NATURE CHEM BIOL. 9(8): 514-20 (2013), incorporated by reference in its entirety.

The HSCs may be, without limitation, primary HSC from one or more donors, an immortalized hepatocyte stellate cell line, cancerous HSCs, or animal-derived HSCs. The HSCs may be in an activated or deactivated/quiescent state.

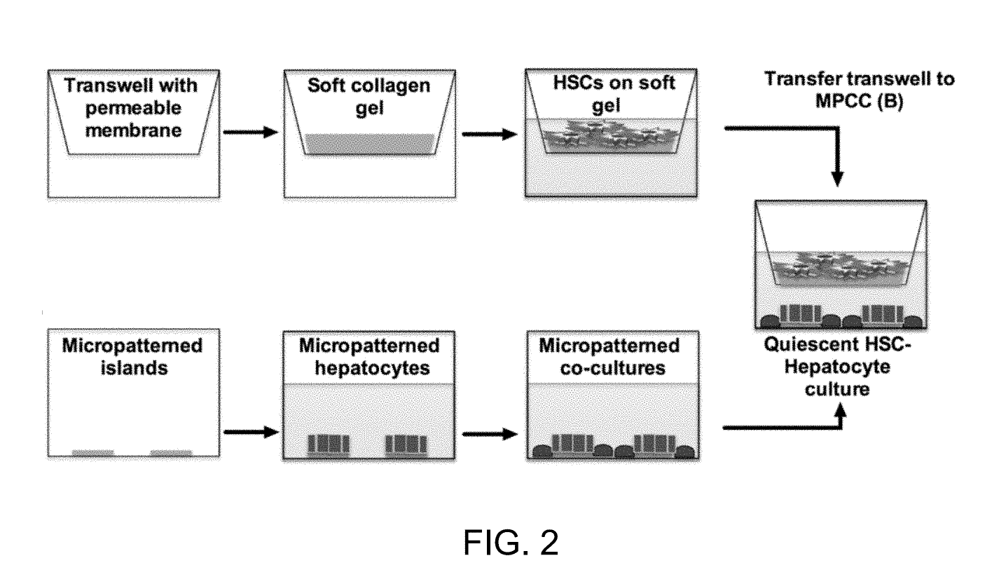

The co-cultures described herein encompass, but are not limited to, randomly distributed co-cultures of hepatocytes, non-parenchymal cells, and HSCs, MPCCs, and hybrids of ECM overlay (“sandwich”) and MPCCs. Co-culturing methods and techniques in general have been described in the literature. In particular, MPCC co-culturing materials, methods and techniques are described detail in Khetani and Bhatia, NATURE BIOTECHNOLOGY, 2008, 26(1):120-126, incorporated by reference in its entirety. The co-cultures may be contact models where the hepatocyte population, at least one non-parenchymal cell population, and HSCs are all contained with the same well. Alternatively, the co-cultures may be paracrine models where transwells containing HSCs on various substrates are transferred to co-cultures of hepatocytes and at least one non-parenchymal cell population as illustrated in FIG. 2.

The culture substrate may comprise a glass or elastomeric structure with a suitable culture surface, such as a glass, polystyrene, or silicon slide, or polystyrene dish, slide or microwells. A biopolymer scaffold may optionally be disposed on the culture substrate to further support and promote cell viability. Biopolymers suitable as scaffold material include, but are not limited to, alginate, chitosan, hyaluronate, fibrous proteins, collagen, silk, and elastin. Alternatively, a scaffold may be disposed on the culture substrate that comprises a hydrogel such as collagens, polyacrylamides, polyelectrolyte multilayers, polydimethylsiloxane The hardness of the substrate may be from about 0.15 KPa to about 60 GPa. Culturing HSCs on a hard/stiff substrate may result in activated HSCs. In some aspects, a hard/stiff substrate may have a hardness of more than 5 KPa to about 60 GPa. In some aspects, a hard/stiff culture substrate may have a hardness of about 1 KPa to about 60 KPa, about 1 KPa to about 20 KPa, about 20 KPa to about 40 KPa, and about 40 KPa to about 60 KPa. In other aspects, the hardness of a hard/stiff substrate may be about 3 GPa to about 20 GPa, about 20 GPa to about 40 GPa, and about 40 GPa to about 60 GPa. In some aspects, a hard/stiff substrate may have a hardness of about 5 to about 20 GPa, about 5 to about 10 GPa, about 10 to about 15 GPa, and about 15 to about 20 GPa. Culturing HSCs on a soft culture substrate results in less activated/quiescent HSCs. In some aspects, a soft substrate may have a hardness of about 0.15 KPa to about 5 KPa. In one aspect, a soft substrate may have a hardness of about 0.15 KPa to about 1 KPa, about 1 KPa to about 2 KPa, about 2 KPa to about 3 KPa, about 3 KPa to about 4 KPa, and about 4 KPa to about 5 KPa.

Co-cultures of the disclosure may be established as randomly distributed co-cultures of hepatocytes, non-parenchymal cells, and HSCs. A co-culture of hepatocytes, non-parenchymal cells, and HSC may be established by seeding all three cell populations at the same time. In other aspects, a culture of hepatocytes may be established first, and then non-parenchyma cells added. In some aspects, HSCs are added with the non-parenchyma cells. In other aspects HSCs, are added to the co-culture after the hepatocytes and non-parenchymal cell co-culture is established.

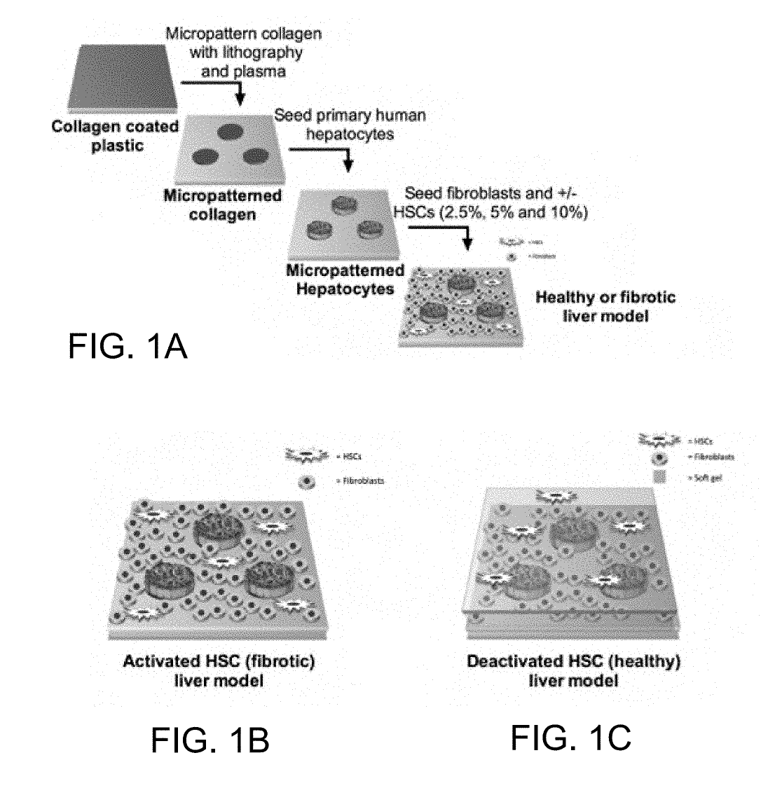

Alternatively, as illustrated in part in FIGS. 1A and 2, the co-cultures may be established according to a micropattern established on the culture surface (MPCC). Micropatterning is not required to create either contact or paracrine co-culture models; however, micropatterning allows for clear demarcation of the hepatocyte islands. The micropattern may comprise for example a predetermined two-dimensional pattern of multiple microdots (“islands”) of the hepatocytes, wherein each microdot has approximately the same microdot diameter and each of any two neighboring microdots shares approximately the same edge-to-edge spacing. While the microdot diameters and microdot spacing may be varied and do vary for cultures with different cell types, it has been found that for hepatocytes, the micropattern may be characterized by microdots each having a diameter of about 500 μm to about 700 μm, and a center-to-center spacing between each microdot of at least about 1000 μm to at least about 1300 μm, including at least about 1100 μm. In some aspects the micropattern may be characterized by microdots each having a diameter of about 500 μm, and an edge-to-edge spacing between each microdot of at least about 1300 μm. A micropattern having the foregoing characteristics has been found to result in co-cultures of hepatocytes that remain viable and show evidence of mature phenotype retention for several days and weeks, including up to at least about 8 days, at least about 28 days, and at least 35 days. (U.S. Patent Publication No. 2015/0240203, incorporated by reference in its entirety.)

In various aspects, each microdot may have a diameter of 10 μm to 100 μm, a diameter of 50 μm to 150 μm, a diameter of 100 μm to 200 μm, a diameter of 150 μm to 250 μm, a diameter of 200 μm to 300 μm, a diameter of 250 μm to 350 μm, a diameter of 300 μm to 400 μm, a diameter of 350 μm to 450 μm, a diameter of 400 μm to 500 μm, a diameter of 450 μm to 550 μm, a diameter of 500 μm to 600 μm, a diameter of 550 μm to 650 μm, a diameter of 600 μm to 700 μm, a diameter of 650 μm to 750 μm, a diameter of 700 μm to 800 μm, a diameter of 750 μm to 850 μm, a diameter of 800 μm to 900 μm, a diameter of 850 μm to 950 μm, and a diameter of 900 μm to 1000 μm.

In various aspects, the microdots may have an edge-to-edge spacing of 200 μm to 300 μm, an edge-to-edge spacing of 250 μm to 350 μm, an edge-to-edge spacing of 300 μm to 400 μm, an edge-to-edge spacing of 350 μm to 450 μm, an edge-to-edge spacing of 400 μm to 500 μm, an edge-to-edge spacing of 450 μm to 550 μm, an edge-to-edge spacing of 500 μm to 600 μm, an edge-to-edge spacing of 550 μm to 650 μm, an edge-to-edge spacing of 600 μm to 700 μm, an edge-to-edge spacing of 650 μm to 750 μm, an edge-to-edge spacing of 700 μm to 800 μm, an edge-to-edge spacing of 750 μm to 850 μm, an edge-to-edge spacing of 800 μm to 900 μm, an edge-to-edge spacing of 850 μm to 950 μm, and an edge-to-edge spacing of 900 μm to 1000 μm.

To establish the micropattern, a cell adhesion molecule may be applied to the culture substrate at the microdots, using for example a PDMS stencil. The cell adhesion molecule is any molecule to which the hepatocytes selectively adhere relative to inter-microdot space, such as collagen, fibronectin, vitronectin, laminin, extracellular matrix proteins, Arg-Gly-Asp (RGD) peptide, Tyr-Ile-Gly-Ser-Arg (YIGSR) peptide, glycosaminoglycans, hyaluronic acid, integrins, ICAMs, selectins, cadherins, cell surface protein-specific antibodies, any combination thereof, and any composition composed substantially of purified extracellular matrix protein, or mixtures of extracellular matrix proteins. Suitable extracellular matrix can be provided for example by ECM derived directly from mammalian liver, such as porcine or human liver. In one micropatterned hepatocyte co-culture, the cell adhesion molecule is for example any of the many extracellular matrix protein products available from a variety of commercial suppliers. In another micropatterned hepatocyte co-culture, the cell adhesion molecule is, for example, a commercially available collagen, such as rat tail collagen.

Following seeding of the hepatocytes onto the micropattern, the non-parenchymal cell population may be seeded onto the culture surface to occupy the inter-microdot space which is not occupied by the hepatocytes. In contact culture models, the HSCs may also be seeded onto the culture surface with the non-parenchymal cell population. Alternatively, the HSCs may be seeded after a co-culture of hepatocytes and non-parenchymal cells has been established.

In some aspects, an overlay may be also be used. The overlay may be disposed on a co-culture of hepatocytes and non-parenchymal cells. The overlay may be disposed on a co-culture of hepatocytes, non-parenchymal cells, and HSCs. Overlays may also be used to enhance hepatic functions. The overlay may be used to restrict HSC group by embedding them into two layers of gel. The co-cultures can be a random co-culture or a MPCC.

In another aspect, as illustrated in FIG. 1C, once the hepatocyte and non-parenchymal cell co-culture has been established, the hepatocyte and non-parenchymal cell co-culture can be overlaid with a gel and HSCs cultured on top of the gel to create a liver model in which the HSCs are not activated to the same degree as on plastic, thereby mimicking a liver closer to a healthy state. The hepatocyte and non-parenchymal cell co-culture can be a random co-culture or a MPCC.

The HSCs may be seeded about one or more hours after the hepatocyte and non-parenchymal cell co-culture is established, about two or more hours after the co-culture is established, about three or more hours after the co-culture is established, about four or more hours after the co-culture is established, about five or more hours after the co-culture is established, about six or more hours after the co-culture is established, about seven or more hours after the co-culture is established, about eight or more hours after the co-culture is established, about nine or more hours after the co-culture is established, about ten or more hours after the co-culture is established, about eleven or more hours after the co-culture is established, about twelve or more hours after the co-culture is established, about thirteen or more hours after the co-culture is established, about fourteen or more hours after the co-culture is established, about fifteen or more hours after the co-culture is established, about sixteen or more hours after the co-culture is established, about seventeen or more hours after the co-culture is established, about eighteen or more hours after the co-culture is established, about nineteen or more hours after the co-culture is established, about twenty or more hours after the co-culture is established, about twenty-one or more hours after the co-culture is established, about twenty-two or more hours after the co-culture is established, or about twenty-three or more hours after the co-culture is established. In some aspects, the HSCs may be seeded about one or more days after the co-culture is established, about two or more days after the co-culture is established, about three or more days after the co-culture is established, or about four or more days after the co-culture is established.

In paracrine co-culture models, HSCs are seeded into transwells with permeable membranes, as shown in FIG. 2. Prior to seeding, the transwell may be adsorbed with extracellular matrix or extracellular matrix gels. HSCs are then seeded onto adsorbed matrix or matrix gels and allowed to attach. Transwells containing HSCs on various substrates are then transferred to micropatterned co-cultures or random co-cultures of hepatocytes and non-parenchymal cells that have been separately established. To make activated cultures of HSCs, the HSCs are seeded on stiff substrates such as plastic or glass. To make less activated/quiescent HSCs, the HSCs are seeded on soft substrates such as soft gels. In paracrine culture models, in contrast to those show in FIG. 1, the hepatocytes and HSCs are not contacting each other, but are communicating with secreted molecules (paracrine signaling). Such a model can allow identification of secreted HSC factors that negatively or positively affect hepatic functions.

Transwells containing HSCs can be transferred to co-cultures of hepatocytes and non-parenchymal cells about two or more hours after the co-culture or HSC culture is established, about three or more hours after the co-culture or HSC culture is established, about four or more hours after the co-culture or HSC culture is established, about five or more hours after the co-culture or HSC culture is established, about six or more hours after the co-culture or HSC culture is established, about seven or more hours after the co-culture or HSC culture is established, about eight or more hours after the co-culture or HSC culture is established, about nine or more hours after the co-culture or HSC culture is established, about ten or more hours after the co-culture or HSC culture is established, about eleven or more hours after the co-culture or HSC culture is established, about twelve or more hours after the co-culture or HSC culture is established, about thirteen or more hours after the co-culture or HSC culture is established, about fourteen or more hours after the co-culture or HSC culture is established, about fifteen or more hours after the co-culture or HSC culture is established, about sixteen or more hours after the co-culture or HSC culture is established, about seventeen or more hours after the co-culture or HSC culture is established, about eighteen or more hours after the co-culture or HSC culture is established, about nineteen or more hours after the co-culture or HSC culture is established, about twenty or more hours after the co-culture or HSC culture is established, about twenty-one or more hours after the co-culture or HSC culture is established, about twenty-two or more hours after the co-culture or HSC culture is established, or about twenty-three or more hours after the co-culture or HSC culture is established. In some aspects, Transwells containing HSCs may be transferred about one or more days after the co-culture or HSC culture is established, about two or more days after the co-culture HSC culture is established, about three or more days after the co-culture HSC culture is established, or about four or more days after the co-culture HSC culture is established.

The HSCs may be seeded or established in transwells at a concentration of about 2.5% to about 10% of the total cells in culture. In some aspects, the HSCs may be seeded or established at concentrations of about 2.5%, about 5%, about 7.5%, or about 10%. In another aspect, the HSCs may be seeded or established at concentrations of about 10% to about 40%, about 10% to about 15%, about 15% to about 20%, about 20% to about 25%, about 25% to about 30%, about 30% to about 35%, and about 35% to about 40% of the total cells in culture. In some aspects, the HSCs may be seeded or established at concentrations of about 15%, about 20%, about 25%, about 30%, about 35%, or about 40% of the total cells in culture.

A biopolymer scaffold may optionally be disposed on the culture substrate to further support and promote cell viability. Biopolymers suitable as scaffold material include, but are not limited to, alginate, chitosan, hyaluronate, fibrous proteins, collagen, silk and elastin and gelatinous protein mixtures. It has also been found that the addition of a Matrigel™ (Corning Life Sciences) layer over the MPCC's increased albumin production and CYP3A4 activity. Matrigel™ is a commercially available gelatinous protein mixture secreted by Engelbreth-Holm-Swarm (EHS) mouse sarcoma cells. A gelatinous extracellular protein mixture may comprise, but is not limited to, collagens and laminins. It should be understood that any gelatinous protein mixture that mimics the extracellular environment found in biological tissues can also be used as the overlay. Additionally, hydrogels may also be used as the overlay.

The co-cultures described herein can be prepared using a culture medium starting with an Eagle's minimal essential medium (EMEM), and a base of Dulbecco's modified Eagle's medium (DMEM) supplemented with about 0.5% to about 10% (vol:vol) bovine (e.g., fetal calf) serum; an insulin-transferrin-selenium (ITS) mixture at about a dilution of about 1:50 to about 1:200; about 0.05 μM to about 1.0 μM dexamethasone; about 0.5 ng/mL to about 20 ng/mL of at least one interleukin-6 cytokine; about 0.5 ng/mL to about 10 ng/mL glucagon; and a B-27® supplement diluted to 1×, or about 1% to about 5%. The medium can further contain about 1% (vol:vol) of an antibiotic or antibiotic mixture, such as penicillin/streptomycin, and a physiological buffering agent, such as HEPES buffer, at about 1.5%. For example, the culture medium can contain, for example, about 1%, about 2%, about 3%, about 4%, about 5%, about 6%, about 7%, about 8%, or about 9% (vol:vol) bovine (e.g., fetal calf) serum. The ITS mixture can be any ITS or ITS+ mixture as known in the art, such as for example insulin-transferrin-selenium-selenous acid. The culture medium can contain the ITS mixture at a dilution of about 1:75, 1:100, 1:125, or 1:150. The culture medium can contain, for example, about 0.1 μM, about 0.2 μM, about 0.3 μM, about 0.4 μM or about 0.5 μM dexamethasone. The interleukin-6 cytokine can be for example any one of IL-6, IL-11, IL-31, cardiotrophin-1 (CT-1), ciliary neurotrophic factor (CNTF), cardiotrophin-like cytokine (CLC), neuropoietin (NP), leptin, leukemia inhibitory factor (LIF), oncostatin M, or any combination thereof. The culture medium can contain, for example, about 1.0 ng/mL, about 1.5 ng/mL, about 2.0 ng/mL, about 2.5 ng/mL, about 3.0 ng/mL, about 3.5 ng/mL, about 4.0 ng/mL, or about 4.5 ng/mL, and so on up to about 20 ng/mL of the at least one interleukin-6 family cytokine. The culture medium can contain, for example, about 1.0 ng/mL, about 2.0 ng/mL, about 3.0 ng/mL, about 4.0 ng/mL, about 5.0 ng/mL, about 6.0 ng/mL, about 7.0 ng/mL, about 8.0 ng/mL, or about 9.0 ng/mL glucagon. A culture medium may comprise for example a DMEM base supplemented with about 1% bovine serum (vol:vol); about 1% (vol:vol) an ITS+ mixture; 0.1 μM dexamethasone; about 2.5 ng/mL oncostatin M; a B-27® supplement at about 2%; about 7 ng/mL glucagon; about 1% penicillin/streptomycin; and about 1.5% HEPES buffer.

The present disclosure also provides a kit for determining the effect of a test agent on hepatocytes. A kit may comprise for example: a population of hepatocytes, at least one non-parenchymal cell population, and a population of HSCs, for preparing one or more MPCC's as disclosed herein. In one aspect, the hepatocytes may be obtained from one or more human donors suffering a disorder of the liver. The kit may further comprise a culture medium as described herein, and/or additional materials or reagents for testing various biological activities of the cells in culture. For example, the kit may contain separately packaged amounts of a glucose-free medium, pyruvate, lactate, glucose, insulin, glucagon, dexamethasone, metformin, a stain or dye such as but not limited to a fluorometric dye, a lipid dye such as Nile red, and/or a cellular stain for glycogen such as PAS stain. The kit may further comprise one or more culture substrates such as a glass, silicon, or polystyrene slide or culture well, and an amount of a cell adhesion molecule. The cell adhesion molecule may be disposed according to a micropattern on the culture substrate as described herein above. Alternatively, the kit may provide an amount of the cell adhesion molecule and a PDMS stencil which can be used together to establish a micropattern as described herein on the culture substrate.

For use with paracrine co-culture models, the kit may further comprise transwells with permeable membranes. The transwell may be adsorbed with extracellular matrix or extracellular matrix gels. Alternatively, the kit may provide an amount of the extracellular matrix or extracellular matrix gel which can be used on the transwells. In other aspects, the kit may provide conditioned medium from cultures of HSCs.

The kit may further comprise a reporter molecule or label capable of generating a signal indicative of a level of a cellular activity of interest in the hepatocytes, such as but not limited a vital dye, a lipid dye, a colorimetric agent, or a bioluminescent marker. The kit may include a detectable label such as a fluorophore, a radioactive moiety, an enzyme, a chromophore, a chemiluminescent label, or the like, and/or reagents for carrying out detectable labeling. The labels and/or reporter molecules, any calibrators and/or controls can be provided in separate containers or pre-dispensed into an appropriate assay format, for example, into microtiter plates.

It is contemplated for example that one or more of the presently disclosed co-cultures can be provided in the form of a kit with one or more containers such as vials or bottles, with each container containing a separate population of cells and/or reagents and washing reagents employed in an assay. The kit can comprise at least one container for conducting the assay, and/or a buffer, such as an assay buffer or a wash buffer, either one of which can be provided as a concentrated solution, a substrate solution for the detectable or a stop solution. The kit may comprise all components, e.g., reagents, standards, buffers, diluents, etc., which are necessary to perform the assay. The kit may contain instructions for determining the presence or amount of any metabolite, biomarker, label, or reporter of interest in the co-culture, in paper form or computer-readable form, such as a disk, CD, DVD, or the like, and/or may be made available online.

Optionally, the kit includes quality control components (for example, sensitivity panels, calibrators, and positive controls). Preparation of quality control reagents is well-known in the art and is described on insert sheets for a variety of immunodiagnostic products. Sensitivity panel members optionally are used to establish assay performance characteristics, and further optionally are useful indicators of the integrity of the immunoassay kit reagents, and the standardization of assays.

The kit can also optionally include other reagents required to conduct a diagnostic assay or facilitate quality control evaluations, such as buffers, salts, enzymes, enzyme co-factors, enzyme substrates, detection reagents, and the like. Other components, such as buffers and solutions for the isolation and/or treatment of a test sample (e.g., pretreatment reagents), also can be included in the kit. The kit can additionally include one or more other controls. One or more of the components of the kit can be lyophilized, in which case the kit can further comprise reagents suitable for the reconstitution of the lyophilized components.

The various components of the kit optionally are provided in suitable containers as necessary, e.g., a microtiter plate. Where appropriate, the kit optionally also can contain reaction vessels, mixing vessels, and other components that facilitate the preparation of reagents or the test sample. The kit can also include one or more instruments for assisting with obtaining a test sample, such as a syringe, pipette, forceps, measured spoon, or the like.

### B. Uses

The hepatocyte co-cultures as described herein may be used in various methods, such as but not limited to drug discovery and drug screening. Such co-culture systems can be used to develop and screen candidate therapeutic agents for treating any hepatic disease or disorder, or for screening the hepatotoxicity of candidate therapeutic agents for treating any other disease or disorder. For example, co-cultures as described herein show the effects of HSCs on hepatocytes and provide for a model of in vitro prediction of drug induced liver toxicity, and establish a new direction in in vitro models of the liver.

A candidate therapeutic agent (also referred to as a “drug candidate”) may be a small molecule, a peptide, a polypeptide, an oligonucleotide, a polynucleotide, or an antibody.

Any of the methods may involve determining a baseline or control value, for example, of any indicator of liver function such as gluconeogenesis, glycolysis, glycogen storage, enzyme activity, albumin secretion, urea production, gene expression, inducible liver enzyme activity and the like, in the hepatocytes in co-culture before administering a dosage of a candidate therapeutic agent or other test agent, and comparing this with a value or level after the exposure and noting any significant change (i.e., greater than the typical margin of experimental error in repeat measurements of the same sample, expressed as one standard deviation from the mean of such measurements) over the control. In non-limiting example, at least one indicator of hepatic function can be a measure of albumin production, urea production, ATP production, glutathione production, enzyme activity, lipid accumulation, liver gene expression, liver protein expression, or inducible liver enzyme activity in the hepatocytes.

### Screening Assays

The present disclosure provides an in vitro model of diseases of the liver, including fibrotic diseases such as non-alcoholic fatty liver disease (“NAFLD”), which can be utilized in various methods for identifying and screening of potential therapeutic agents, and for drug development.

For example, the compositions of the present disclosure may be used in vitro to screen a wide variety of compounds, such as small molecules, antibodies, peptides, polypeptides, nucleic acid-based agents and the like, to identify therapeutic agents having a therapeutic effect on liver function in any disease or disorder of the liver, and/or to assess the toxicity of any such therapeutic agent before clinical implementation. For example, following contact of a co-culture with a candidate therapeutic agent, various cellular functions in the hepatocytes and/or HSCs may be assessed by examining gene expression, albumin production, urea production, cytochrome P450 (CYP) metabolic activity or any inducible liver enzyme activity, uptake and secretion of liver-specific products, and response to hepatotoxins, by detecting and/or measuring level of a protein, metabolite, reporter molecule, label, or gene expression level such as through gene fluorescence in the cell or in the culture media. In non-limiting example, at least one indicator of hepatic function can be, for example, albumin production, urea production, ATP production, glutathione production, enzyme activity, lipid accumulation, liver gene expression, or liver protein expression in the hepatocytes.

Gluconeogenesis and other liver functions such as albumin secretion, urea production, and glycolysis and glycogen storage may be monitored in the presence and absence of one or more stimuli, test agent, or candidate therapeutic agent. For example, hepatocytes in co-culture as described herein may be tested for any one or more of albumin secretion, urea production, ATP production, lipid accumulation, induction of inducible liver (e.g., CYP) enzyme levels, gluconeogenesis, glycolysis and glycogen storage in the presence and absence of varying levels of candidate therapeutic agents. In any method involving measurement of one or more inducible liver enzymes, such enzymes include, in non-limiting example, CYP enzymes such as CYP2C9 (luciferin-H), CYP3A4 (luciferin-IPA), a combination of CYP1A1, CYP1A2, CYP2B6 and CYP2D6 (luciferin ME-EGE), all CYP450 enzymes such as CYP2C8, CYP2C19, CYP2E1, and phase II enzymes such as UGTs, SULTs and NATs, and any combination thereof.

Levels of biomarkers such as for example specific metabolites may also be used in screening assays for agents. This may also be accomplished using standard cytological and/or histological techniques including the use of immunocytochemical techniques employing stains that recognize specific cellular components such as lipids, or antibodies that specifically bind to biomarkers with antigenic activity. For example, co-cultures of the present disclosure may be exposed to a test agent or candidate therapeutic agents. After incubation, the co-cultures may be examined for change in biomarker production as an indication of the efficacy of the test substance. Varying concentrations of a candidate therapeutic agent may be tested as known in the art to derive a dose-response curve.

### Target Validation

The compositions of the invention can be used in drug development for specific target identification and target validation. The disclosed co-cultures are useful for identifying targets and predicting the role of one or more biomolecules in liver function in a disease or disorder of the liver. A “disease or disorder of the liver” is any medical condition having a negative effect on any liver function. Non-limiting examples of liver diseases and disorders include cirrhosis, diabetes, fibrosis, any chronic hepatitis (including but not limited to A, B, C, D, and E), non-alcoholic fatty liver disease (“NAFLD”), alcoholic fatty liver, tumors of the liver such as hepatic carcinoma, and genetic disorders such as alpha-1-anti-trypsin deficiency.

For example, the cultures and systems may be used to identify proteins playing a potential role in fibrosis of the liver, or those playing a potential role in diabetic processes or diabetic liver pathways. Identified proteins may be modulated (e.g., up-regulated or down-regulated) in the co-cultures described herein, and processes and pathways related to diabetes may be assayed following modulation.

The co-cultures and systems are also useful for validating the predicted role of one or more biomolecules in liver function in a disease or disorder of the liver. For example, proteins identified in preliminary studies (e.g., studies of primary hepatocytes in conventional culture systems or cryogenically preserved hepatocytes, studies in other liver models, differential expression studies, etc.) as playing a potential role in disease processes or disease pathways can be tested in a composition as described herein to confirm the potential role. Proteins identified from preliminary studies, for example proteins suspected to play a role in diseased or disordered liver function, may be modulated (e.g., up-regulated or down-regulated) in the co-cultures described herein, and processes and pathways related to the disease or disorder may be assayed following modulation. For example, candidate proteins can be “knocked out/down” using gene knockout or suppression techniques, for example, using various genomic editing techniques, or the introduction of RNA interference (RNAi) agents. Inhibition of liver pathways may be tested following down-regulation and candidate proteins thought to be important in disease or disordered liver function may be thus validated.

Any method using the co-cultures as disclosed herein may comprise initially preparing or otherwise obtaining a micro-patterned co-culture of hepatocytes, non-parenchymal cells, and HSCs as described herein. In one aspect, a stable, growing co-culture is established having a desired size (e.g., island size and distance between islands) as described herein above. In one aspect, the present disclosure provides a method of determining the efficacy of a candidate therapeutic agent for treating a disease or disorder of the liver. The candidate therapeutic agent may be a small molecule, a peptide, a polypeptide, an oligonucleotide, a polynucleotide, or an antibody.

The co-culture is exposed to varying concentrations of the candidate therapeutic agent. The amount of the candidate therapeutic agent may be, according to knowledge available to those of skill in the art, an amount representing a proposed dose or range of proposed doses in a clinical population. The time over which the hepatocytes in the co-culture are exposed to the candidate therapeutic agent may be, according to knowledge available to those of skill in the art, a period of days, weeks, or months representing time course of exposure in a clinical population. After incubation with the agent, the culture is examined to determine impact of the agent if any on one or more target biomolecules or pathways identified as potentially involved in liver function in a disease or disorder of the liver, as described above. Once a testing range is established, varying concentrations of the agent can be tested to determine therapeutically effective amount of the test compound.

As noted above, the hepatocytes and/or HSCs can be obtained or derived from human donors. In further aspects, hepatocytes and/or HSCs can be derived from stem cells obtained from one or more human donors. In another aspect, the hepatocytes and/or HSCs can be obtained from one or more human donors suffering from a disease or disorder of the liver. Alternatively, the hepatocytes and/or HSCs can be derived from stem cells obtained from one or more human donors suffering from a disease or disorder of the liver.

By way of example, stem cell derived hepatocytes and/or HSCs can be derived from stem cells obtained from one or more human donors suffering from a metabolic disorder of the liver, such as NALFD. The methods therefore encompass, for example, a method for testing a candidate therapeutic agent for treating a disorder of the liver, including maintaining a co-culture as described herein for a time and under conditions sufficient to allow glucose production by the hepatocytes; and determining a level of glucose production by the hepatocytes, wherein the level of glucose production relative to the level of glucose production in a population of control stem cell derived hepatocytes is indicative of the efficacy of the test compound as an therapeutic agent for treating the disorder of the liver. The method may further comprise, prior to determining the level of glucose production by the hepatocytes: depleting the co-culture of glycogen in glucose-free medium for a period of at least about twelve hours; contacting the co-culture with at least one substrate of a gluconeogenesis enzyme; and maintaining the co-culture for a period of at least about 12 hours under conditions sufficient for glucose production in the hepatocytes to occur. The co-culture may be maintained for a period of at least about 24 hours, or at least 48 hours under conditions sufficient for glucose production in the hepatocytes to occur. The at least one substrate of a gluconeogenesis enzyme may be for example lactate or pyruvate.

It should be understood that the present disclosure encompasses methods of identifying any test agent useful for modulating a biological activity of interest in a hepatocyte or HSC, in which a co-culture as disclosed herein is contacted with the test agent; the co-culture is maintained for a time and under conditions sufficient for the hepatocytes to generate a signal indicative of the biological activity; and a signal generated by the hepatocytes in the presence of the test agent is detected, wherein the signal relative to a signal generated in a control hepatocyte in a control co-culture is indicative of an effect on the biological activity of interest in the hepatocytes. The signal indicative of the biological activity of interest may be for example a protein expression level or a protein secretion level. The biological activity of interest may be glucose metabolism. The biological activity of interest may be albumin secretion or urea synthesis.

### Toxicity Studies

In addition to the above-described uses of the cultures and/or systems of the invention in screening for therapeutic agents for treating a disease or disorder of the liver, the co-cultures may also be used in toxicology studies to determine the hepatotoxicity of an agent identified as a potential therapeutic agent. Toxicology studies may be performed on co-cultures featuring hepatocytes from human donors suffering from a disease or disorder of the liver, as described herein, which may be contrasted with comparable studies in cells from a different source. The co-cultures described herein may be used in vitro to test a variety of potential therapeutic compounds for hepatotoxicity. Any of the screening methods described herein above may further comprise determining the toxicity of the agent by measuring in the hepatocytes at least one cell signal indicative of cell toxicity.

Toxicity results may be assessed for example by observation of any of the following: a change in albumin and/or urea production, induction of any inducible liver enzyme such as cytochrome P450 (CYP) enzymes, cellular necrosis, loss of membrane integrity, cell lysis, decrease in cell viability, apoptosis, mitochondrial membrane potential, mitochondrial DNA, ER stress, and steatosis, using any one or more of vital staining techniques, ELISA assays, RT-qPCR, immunohistochemistry, and the like or by analyzing the cellular content of the culture, e.g., by total cell counts, and differential cell count, by metabolic markers such as MTT and XTT, or by hepatocyte imaging technology (HIAT).

For example, co-culture as described herein are exposed to varying concentrations of a candidate therapeutic agent. The amount of the candidate therapeutic agent may be, according to knowledge available to those of skill in the art, an amount representing a proposed dose or range of proposed doses in a clinical population. The time over which the hepatocytes are exposed to the candidate therapeutic agent may be, according to knowledge available to those of skill in the art, a period of days, weeks, or months representing time course of exposure in a clinical population. After incubation with the agent, the culture is examined to determine the highest tolerated dose, i.e., the concentration of the agent at which the earliest morphological and/or functional abnormalities appear or are detected. Cytotoxicity testing may also be performed using a variety of supravital dyes to assess cell viability in the culture system, using techniques known to those skilled in the art. Once a testing range is established, varying concentrations of the agent can be examined for hepatotoxic effect.

The present disclosure thus provides a method for determining the cellular toxicity of a candidate therapeutic agent or test compound, the method comprising contacting a co-culture as described herein with the test compound; maintaining the co-culture for a time and under conditions sufficient to allow an effect of the test compound on the hepatocytes and/or HSCs; and taking a test measurement and/or otherwise obtaining test data indicative of a negative impact of the test compound on hepatocytes and/or HSCs, which is indicative of toxicity of the test compound. The test measurement can be any measurement which provides an indicator of hepatic cell or HSC function. For example, the test measurement can be a measurement of at least one or any combination of albumin, urea, enzyme activity, lipid accumulation, ATP production, and gene expression. The test measurement can be a measurement of at least one inducible liver (e.g., CYP) enzyme level. Test data may include applying hepatocyte imaging technology (HIAT) to the hepatocytes and/or HSCs to obtain a test image. Test data may include using other imaging technology on the co-cultures, hepatocytes, and/or HSC to obtain a test image. The test measurement and/or test image is compared to a control measurement or control image from the hepatocytes and/or HSCs before contact with the test compound, and a difference between the test measurement and control measurement, or between test image and control image is indicative of toxicity of the test compound. For example, a relative decrease in albumin and/or urea production in test measurements as compared to control, following exposure of the co-culture to the test compound is indicative of hepatotoxicity. A relative increase in inducible CYP enzyme test measurements as compared to control, following exposure of the co-culture to the test compound is indicative of hepatotoxicity. A relative change in vitamin A, desmin, glial fibrillary acidic protein (GFAP), Yes-associated protein 1 (YAP1), IL6 secretion, collagen I deposition, and activation markers (for example, but not limited to, lysl oxidase (LOX) and collagen I (COL1A1)), compared to control, following exposure of the co-culture to the test compound is indicative of a change in state of function (for example, quiescence or activation) in HSCs.

The present disclosure also provides a method of determining the toxicity arising from a drug interaction. For example, the potential toxicity of an interaction between a first test compound and a second test compound can be examined by contacting a co-culture as described herein with the first and second test compounds; maintaining the co-culture for a time and under conditions sufficient to allow an effect of an interaction between the first and second test compounds on the hepatocytes; and taking a test measurement and/or otherwise obtaining test data as described above, which is indicative of toxicity of the interaction of the first and second test compounds.

The present disclosure also provides a method of determining whether a test compound alleviates hepatic dysfunctions cause by HSCs. For example, the effects of a test compound can be examined by contacting a co-culture as described herein with the test compound; maintaining the co-culture for a time and under conditions sufficient to allow an effect of the test compound on the hepatocytes and HSCs; and taking a test measurement and/or otherwise obtaining test data as described above, which is indicative of effect of test compounds on hepatic function. In some aspects more than one test compound can be examined at one time.

Additionally, the present disclosure thus also provides a method for determining the effects of chronically elevated or reduced levels of glucose, fructose and/or fatty acids on the liver and liver function. The method comprises for example contacting a co-culture as described herein with a predetermined amount of one or more metabolites such as glucose, fructose, and or fatty acids, wherein the hepatocytes or HSCs are obtained from one or more human donors suffering from a disorder of the liver; maintaining the co-culture for a time and under conditions sufficient for the hepatocytes to generate a signal indicative of modified cellular function induced by the predetermined amount of one or more metabolites; and detecting the signal generated by hepatocytes in the presence of the one or more metabolites, wherein the signal relative to a signal generated in a control cell subject to the same conditions is indicative of an effect of the amount of the one or more metabolites on the hepatocytes. The signal indicative of an effect on cell function may be a change in transcription, translation or secretion of a protein, cellular necrosis, loss of membrane integrity, cell lysis, decrease in cell viability, apoptosis, mitochondrial membrane potential, mitochondrial DNA, ER stress, and steatosis. The predetermined amount may be an amount which is elevated or reduced relative to a control amount which is representative of an amount of each metabolite which is considered within the range of normal in vivo values for the metabolite. The time over which the hepatocytes are exposed to the elevated or reduced level(s) of metabolite(s) may be, according to knowledge available to those of skill in the art, a period of days, weeks or months representing chronic elevation or reduction of the metabolite in a clinical population.

It should be understood that many other signals of toxicity from the hepatocytes and HSCs can be detected and/or measured and compared to controls to identify and/or quantify toxicity induced by a candidate therapeutic agent, wherein the signal relative to a signal generated in a control co-culture is indicative of a toxic effect of the candidate agent on the hepatocytes and/or HSCs. Such signals include, in non-limiting example, cellular necrosis, loss of membrane integrity, cell lysis, decrease in cell viability, apoptosis, mitochondrial membrane potential, mitochondrial DNA, ER stress, and steatosis, any one of which can be readily measured using techniques and materials known in the art.

It will be readily apparent to those skilled in the art that other suitable modifications and adaptations of the methods of the disclosure described herein are obvious and may be made using suitable equivalents without departing from the scope of the disclosure or the embodiments disclosed herein. Having now described the present disclosure in detail, the same will be more clearly understood by reference to the following examples, which are included for purposes of illustration only and are not intended to be limiting of the disclosure.

### Personalized Medicine

It has been found that certain correlations can exist between an individual subject's particular genotype with respect to specific molecular markers, and drug treatment efficacy. Any of the co-cultures and methods described herein can also be used to develop personalized medicine, to determine whether any such correlation exists between a particular genotype and selected drug treatment for a disease or disorder of the liver. For example, co-cultures can be prepared using hepatocytes derived from pluripotent stem cells obtained from a variety of donors of different genotypes, and any therapeutic candidate can be tested for efficacy against each genotype to determine whether any one or subset of the tested genotypes fares better or worse with a given therapeutic candidate. Any therapeutic candidate can be tested for effect on any inducible liver enzymes, and/or for a negative interaction with a second therapeutic candidate. Such information considered together with the genotype of an individual patient, can be used by a health care provider to determine a treatment option with the highest likelihood of efficacy for the individual subject, and/or to determine a risk of a negative side effect in the individual subject from a therapeutic candidate.

### EXAMPLES

The following examples are provided for representative guidance to make and use the compositions according to the inventive subject matter. However, it should be recognized that numerous modifications may be made without departing from the inventive concept presented herein.

HSCs, the vitamin A storing pericytes of the liver, normally aid in tissue regeneration while they contribute to fibrosis, and hepatocellular carcinoma progression in disease. HSCs have a quiescent phenotype in the healthy liver, but upon stimulation they become activated and secrete various molecules (extracellular matrix, MMPs, TIMPs, growth factors and cytokines) that can engender the organ to an altered functional state. Some diseases associated with activated HSCs include viral infection, drug induced liver injury, and non-alcoholic fatty liver disease (NAFLD), which affects ˜30% of the general population. Non-alcoholic steatohepatitis (NASH) is the progressive stage of NAFLD characterized by not only fatty liver, but also inflammation and fibrosis. The increasing prevalence of NASH (5-10% of NAFLD patients) and lack of treatment options makes this disease the second most common etiology for liver transplantation. Hepatocytes are negatively affected at the functional level in NASH where they become steatotic, swelled (ballooned), and display altered cytochrome P450 enzyme activity as well as transport in and out of the liver.

The latter two symptoms make drug metabolism unpredictable, but targeting fibrosis, nuclear receptors, inflammation, and oxidative stress pathways have yielded positive results for NASH regression in animal models, but not always clinical trials. The key to the successful translation of treatments from pre-clinical scenarios to actual patient therapies could lie in the development of human relevant disease models. Fibrosis, not inflammation, has recently been identified as the major predictor for long-term outcomes in NASH patients. Non-hepatocyte/immune cell stimuli such as tissue compliance and hormonal/nutrient signaling can activate HSCs. Specifically, increased tissue/substrate stiffness, insulin, and a loss of vitamin A correlate with the activation of HSCs in vitro and in vivo. These parameters are hallmarks of fibrosis, NASH, and obesity.

Once activated, HSCs can contribute to inflammation by secreting proinflammatory cytokines such as interleukin 6 (IL-6) and tumor necrosis factor alpha (TNFα). Therefore, prior to inflammation onset, fibrosis or even lipid accumulation, HSCs in obese patient livers could contribute to NAFLD progression. However, the effects of activated HSCs on hepatocytes have scarcely been addressed. Since hepatocytes rapidly dedifferentiate in vitro, most hepatocyte-stellate cell studies have focused on the ability of HSCs to retain hepatocyte phenotype. Unfortunately these experiments use declining pure hepatocyte cultures as controls, which can make HSC-hepatocyte interactions look beneficial, while the effects of HSCs might actually be detrimental to stable healthy hepatocytes. HSCs have clearly been shown to have negative effects on hepatocytes in vivo and in vitro, therefore, highly differentiated, stable hepatocyte cultures are needed to properly study HSC-hepatocyte interactions over time. Accordingly, primary human HSCs were integrated into an engineered long co-culture model to make a co-culture for addressing the question of how activated HSCs could contribute to liver disease associated symptoms with prolonged exposure.

### Example 1. Primary Hepatic Stellate Cells Support Long-Term Hepatocyte Functions at Physiologically Relevant Concentrations

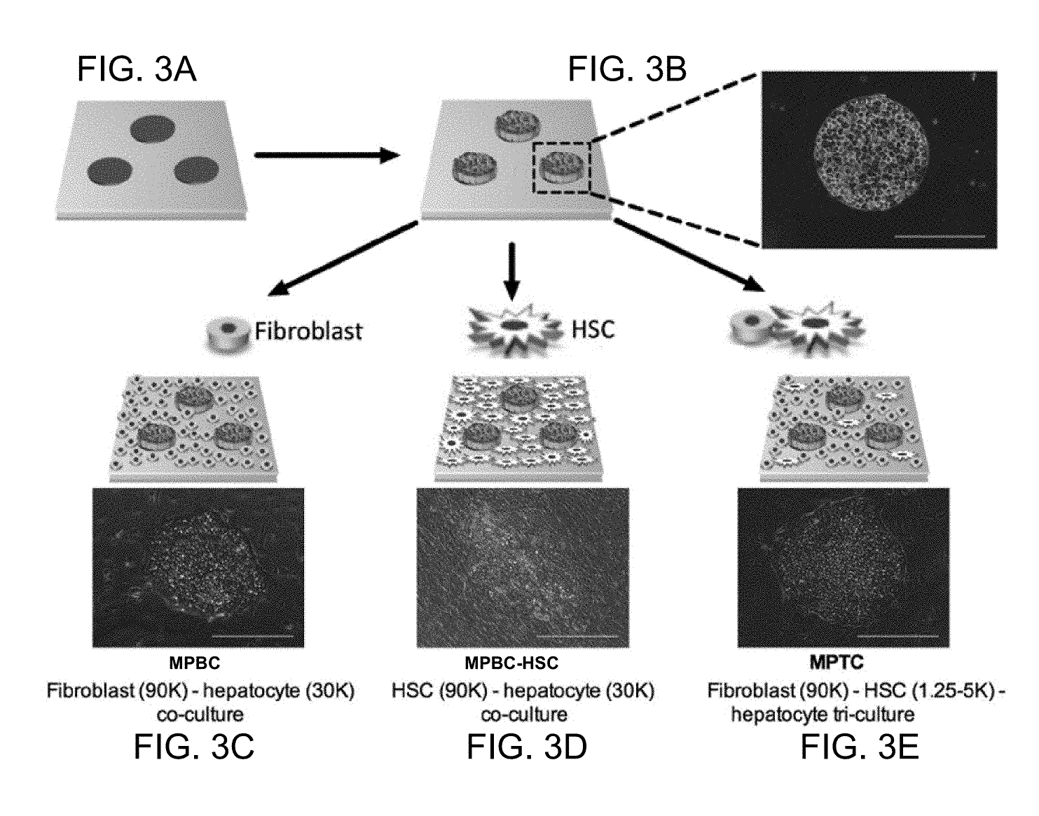

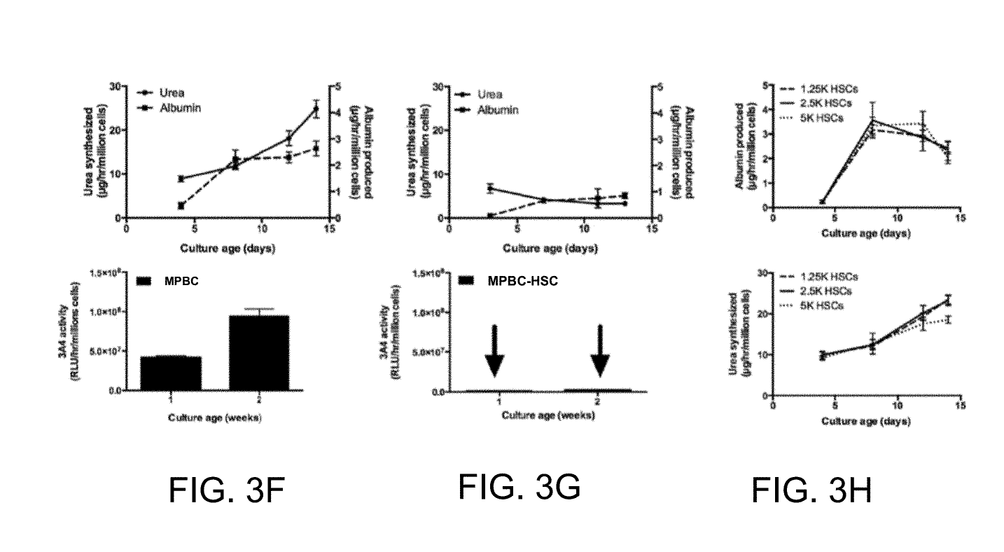

For preparation of direct contact co-culture models, cryopreserved primary human hepatocytes (“PHHs”) were obtained from companies certified to distribute human origin tissues in the US permitted by Organ Procurement Organizations (Triangle Research laboratories, Durham, N.C.). The cryopreserved PHHs were seeded onto micropatterned collagen islands on day 0 (March et al., NAT PROTOC 2015; 10, 2027-2053) (FIG. 1A; FIG. 3A and FIG. 3B). For pure culture studies, hepatocytes were seeded on collagen coated TCPS at a density of 1.05e⁶ cells/cm².

Collagen gels were fabricated using 8.69 mg/ml high density type I rat tail collagen (Corning) and 10×PBS (Corning) at a neutral pH as previously described (Yang, Y. L, et al., Elastic moduli of collagen gels can be predicted from two-dimensional confocal microscopy. BIOPHYSICAL JOURNAL 97, 2051-2060 (2009), incorporated by reference in its entirety). Briefly, 10×PBS, 1N NaOH, 1M NaCl and molecular grade H₂O were combined and placed on ice and then mixed with 0.23 ml of 8.69 mg/ml acid solubilized rat tail collagen to achieve a 1×PBS, 0.005M NaOH, 0.025M NaCl, 2 mg/ml collagen, ˜pH 7 solution. This solution was subjected to 3, 1 second pulses on a vortex, and then pipetted into culture dishes (250 μl/well on a 24 well plate) and allowed to polymerize for at least 1 hour at 37° C. Gels were then washed 3× with warm culture medium and left at 4° C. until use (within 24 hours of fabrication).

Twenty-four hours (day 1) after seeding the cryopreserved PHHs, growth arrested (1 μg/ml mitomycin C in fibroblast maintenance medium for 4 hours) fibroblasts (3T3-J2 murine embryonic fibroblasts) (FIG. 3C (MPBC)), primary HSCs (FIG. 3D (MPBC-HSC)), or fibroblasts and primary HSCs (passage 1-4) (FIG. 3E (MPTC)) were seeded at various densities into wells containing micropatterned hepatocytes.

3T3-J2 murine embryonic fibroblasts were maintained in high glucose DMEM with 10% bovine serum (Life Technologies), and 1% penicillin/streptomycin solution. For co-culture experiments, 3T3-J2s below passage 12 were used.

Cryopreserved stellate cells were obtained from companies certified to distribute human origin tissues in the US permitted by Organ Procurement Organizations (Sciencell Research Laboratories, Carlsbad, Calif. (stellate cells), Zen-Bio Inc, Durham, N.C. (stellate cells)). Human cell work was carried out at Colorado State University and the University of Illinois at Chicago with the approval of the Institutional Biosafety Committee. HSCs were seeded on to poly-L-lysine (PLL) (Sciencell) coated flasks (20 μg/ml) and passaged (1-3 times) in Sciencell stellate cell medium. One passage prior to seeding into MPBC or MPTC with hepatocytes, HSCs were activated by culturing in 10% FBS (Life Technologies, Carlsbad, Calif.) with high glucose DMEM (Corning, Manassas, Va.), 1% penicillin/streptomycin (Corning) and 15 mM HEPES (Corning) on PLL coated flasks. HSCs were labeled with a fluorescent PKH 67 membrane dye (Sigma Aldrich, St. Louis, Mo.) after trypsinization, using manufacture protocol, prior to seeding into cultures.

Pure HSC cultures, on a collagen coat (100 μg/ml) or on gels, were seeded at a density of 2.63e³ cells/cm², while co-culture studies were seeded at a density of 47.37e³ cells/cm², and tri-culture experiments were seeded at physiologic ratios to the number of hepatocytes/well. It was assumed that hepatocytes make up ˜60% of the cells in the liver, and that there were 20-30 thousand hepatocytes/well. HSCs should make up ˜5% of the cells in the liver, or 2.5 thousand HSCs/well. Specifically, 2.5% HSCs were seeded at 0.66e³ cells/cm², 5% HSCs were seeded at 1.32e³ cells/cm² and 10% HSCs were seeded at 2.63e³ cells/cm², simultaneously with 3T3-J2s.

Cell culture medium consisted of 10% bovine serum (Life Technologies), 1% ITS+ Premix (Corning), 1% penicillin/streptomycin (Corning) and 15 mM HEPES (Corning), 5 mM glucose (Thermo Fisher Scientific, Waltham, Mass.), 100 nM dexamethasone (Sigma Aldrich) and 2 nM glucagon (Sigma Aldrich).

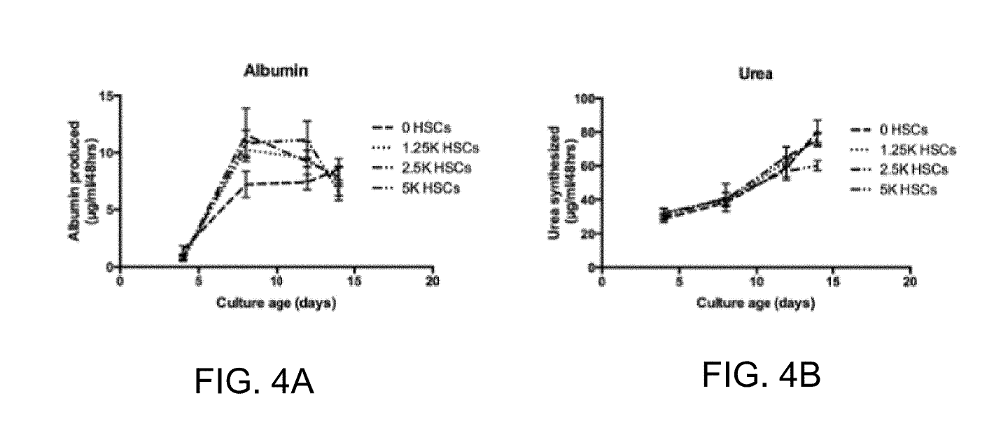

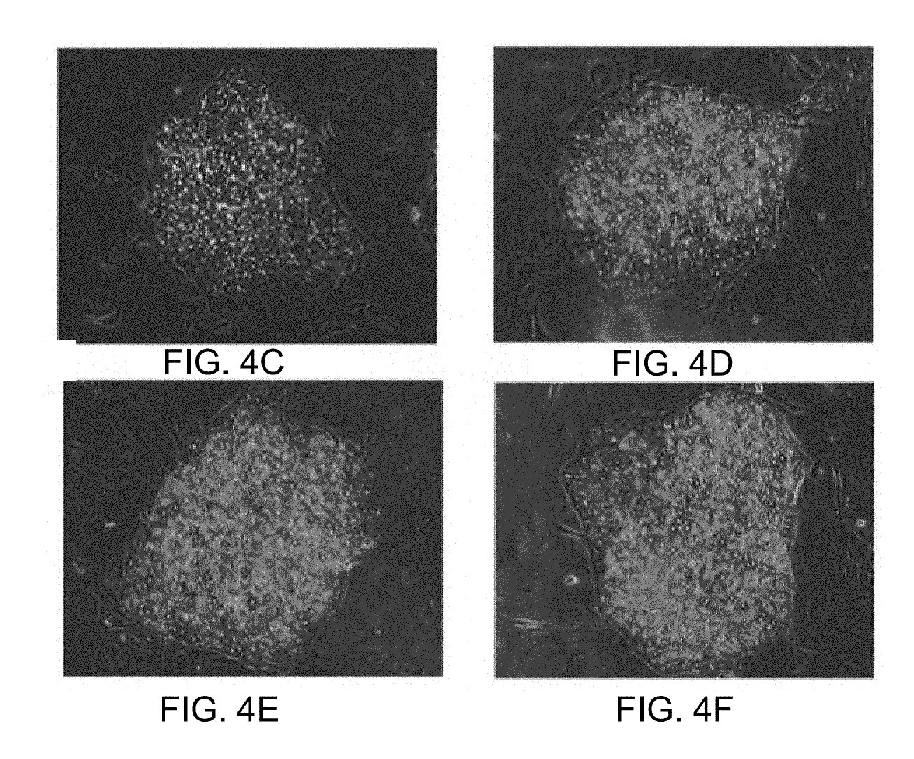

The fibroblasts and HSCs surround the hepatocyte “islands.” Phase contrast images demonstrate hepatocyte morphology in MPBC cultures (hepatocytes-fibroblasts) versus MPTC cultures (hepatocytes-fibroblasts-HSCs) (FIG. 4C-F). In the presence of varying amounts of HSCs (FIG. 4D-F) relative to the absence of HSCs (FIG. 4C), lipid accumulation is observable where HSCs are present. This lipid accumulation also occurs in pure hepatocyte cultures cultured on gels to sustain phenotype.

Basic hepatocyte functions, albumin and urea production, in the three different culture setups, MPBC, MPBC-HSC or MPTC (with a set number of fibroblasts, 90 K, and a range of HSC seeding densities 1.25 K-5 K per 1.9 cm²) were measured.

Urea and albumin in culture supernatants were quantified using a colorometric acid based detection method and competitive ELISA, respectively, as previously described (Davidson, et al., Hormone and Drug-mediated Modulation of Glucose Metabolism in a Microscale Model of the Human Liver. TISSUE ENGINEERING PART C: METHODS 141217055135006 (2014) doi:10.1089/ten.TEC.2014.0512, incorporated by reference in its entirety).

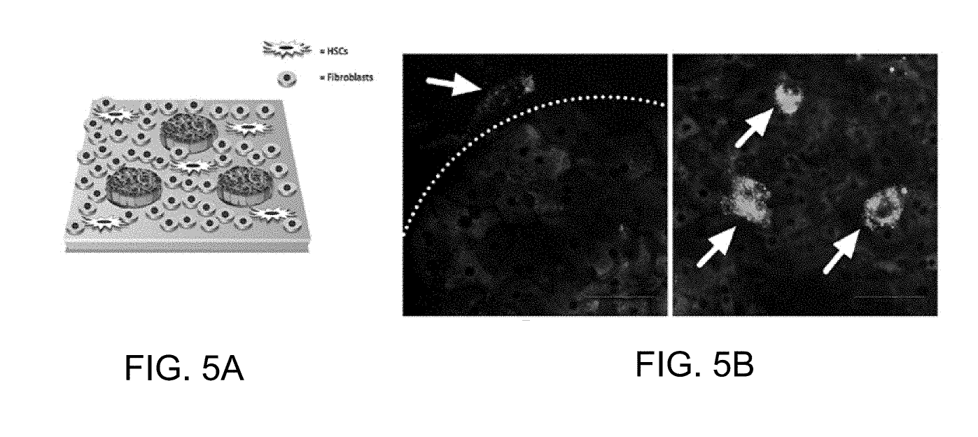

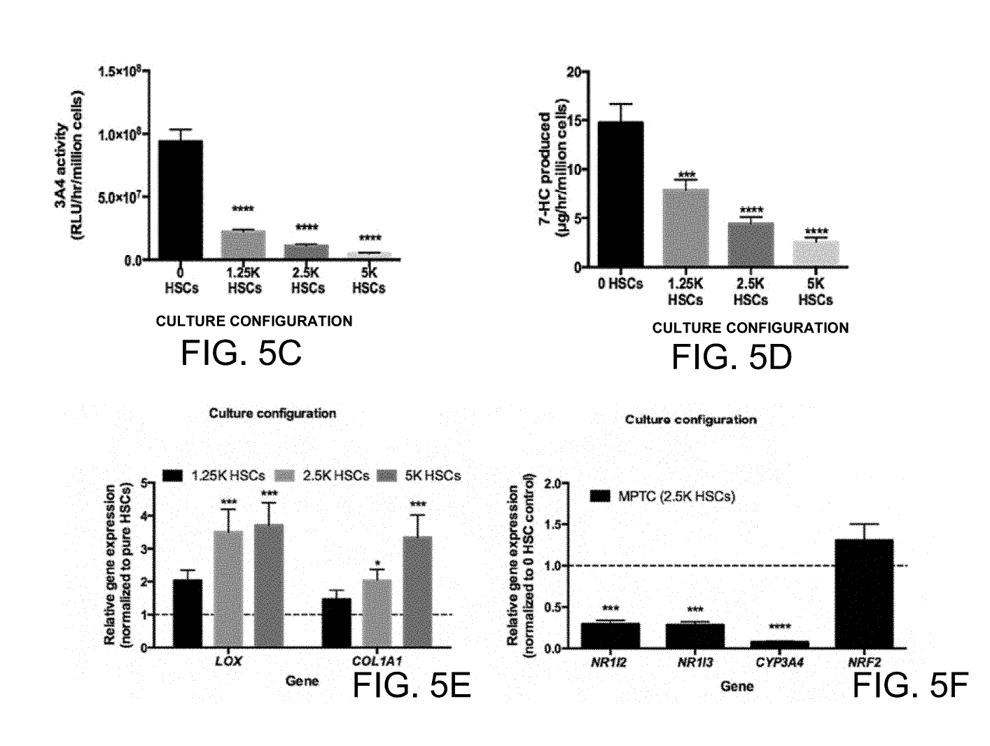

MPBCs supported steady high levels of albumin and urea over 2 weeks of culture (FIG. 3F) while MPBC-HSCs failed to support high levels of these hepatocyte markers (FIG. 3G). CYP 3A4, which metabolizes ˜50% of all pharmaceuticals prescribed, activity, measured by the production of luciferin from luciferin-IPA, was supported and stable over time in MPBCs (FIG. 3F), while 3A4 activity was diminished in MPBC-HSCs (FIG. 3G). Seeding primary HSCs at lower ‘physiologic’ densities, when compared to hepatocyte number (˜20-30 K/well), supported hepatocyte albumin (FIG. 3H) and urea (FIG. 3H) production over 2 weeks of culture. Even at these low densities, PKH67 pre-labeled primary HSCs (green) were visible (FIG. 5B) around and on top of hepatocyte islands stained for mitochondrial membrane potential with tetramethylrhodamine methyl ester ((TMRM) magenta)). A repeat experiment supported the finding that cultures comprising HSCs relative to cultures without HSCs supported hepatocyte albumin (FIG. 4A) and urea (FIG. 4B) production over two weeks of culture. Taken together, this data shows that the presence of activated HSCs led to changes in hepatocyte lipid profile without affecting albumin and urea secretion from the hepatocytes.

Of note, micropatterning is not required to create the tri-culture model, however, micropatterning allows for clear demarcation of the hepatocyte islands for potential high content imaging and automated tracking of hepatic markers.

### Example 2. Activated Stellate Cells Down Regulate Drug Metabolism Pathways in Primary Human Hepatocytes

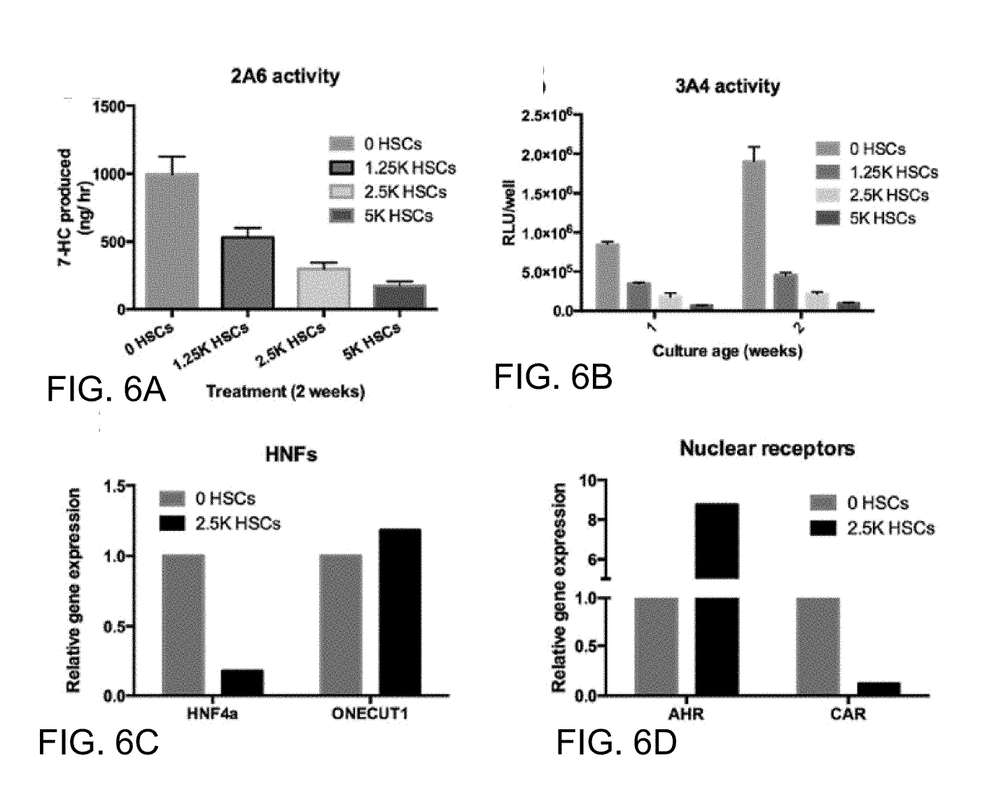

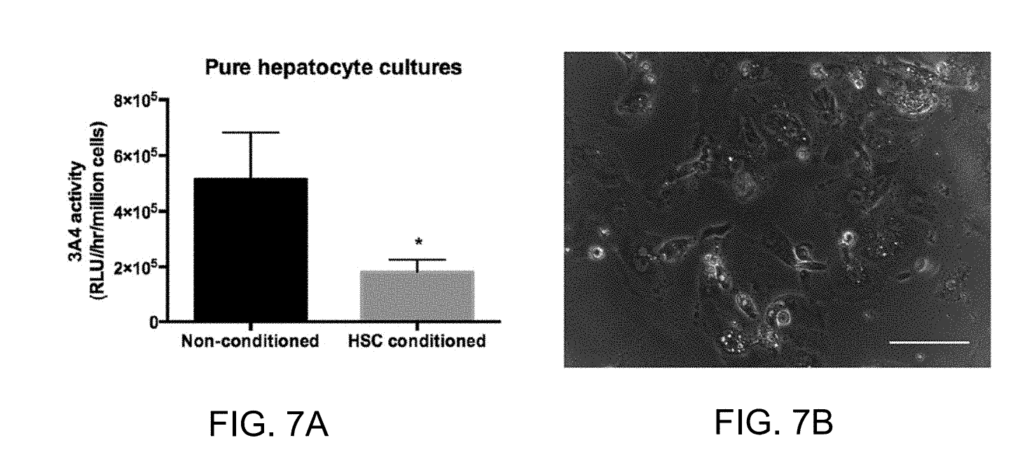

When CYP 3A4 activity was assessed in MPTCs, there was a significant HSC density dependent decrease in enzyme activity at 2 weeks of culture (this trend was confirmed in 3 hepatocyte donors and 2 HSC donors) when compared to the same MPBC shown in FIG. 3F (see FIG. 5C and FIG. 6B). HSC conditioned medium also down regulated pure hepatocyte CYP 3A4 activity, but these cultures rapidly dedifferentiated, which limits their use for long term studies (see FIG. 7A). CYP 2A6 activity, as measured by the amount of coumarin carbon-7-hydroxylation in 1 hour, was also significantly decreased (FIG. 5D and FIG. 6A) in an HSC density dependent manner when compared to an MPBC control. CYP 3A4 and 2A6 activity was assessed by washing cultures once with serum free medium, and then incubating with 3 μM luciferin-IPA (3A4) (Promega, Madison, Wis.) or 50 μM coumarin (2A6) (Sigma-Aldrich, St. Louis, Mo.) in serum free medium for 1 hour.

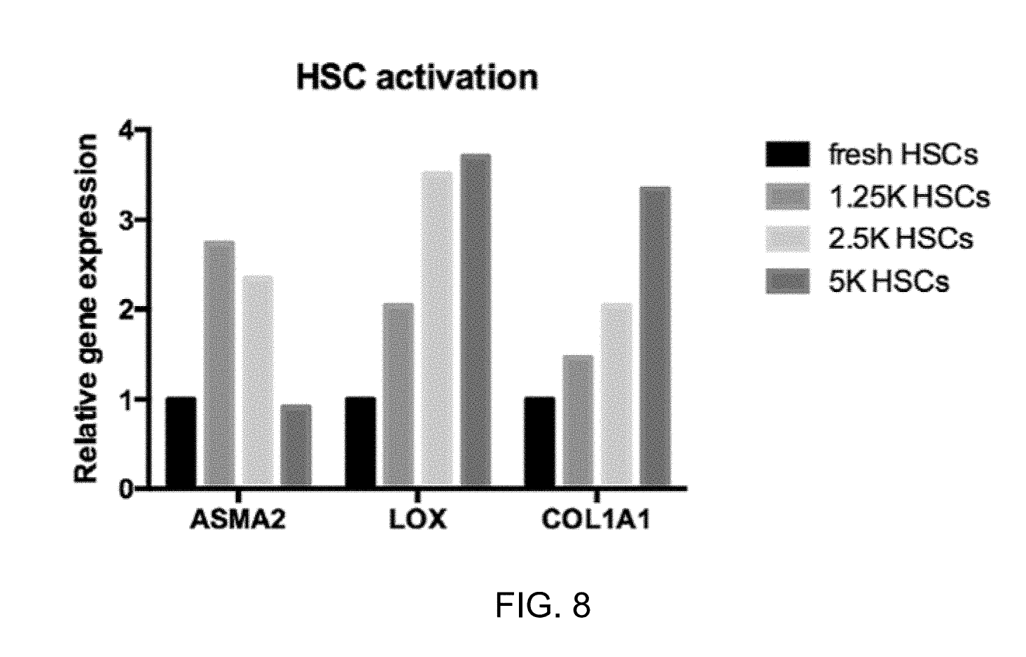

MPTC gene expression for HSC activation/fibrosis markers, lysyl oxidase (LOX) and collagen I (COL1A1), was significantly increased (FIG. 5E and FIG. 8) in an HSC density dependent manner. MPTCs from here onward will refer to MPTCs with 2.5 K HSCs unless noted.

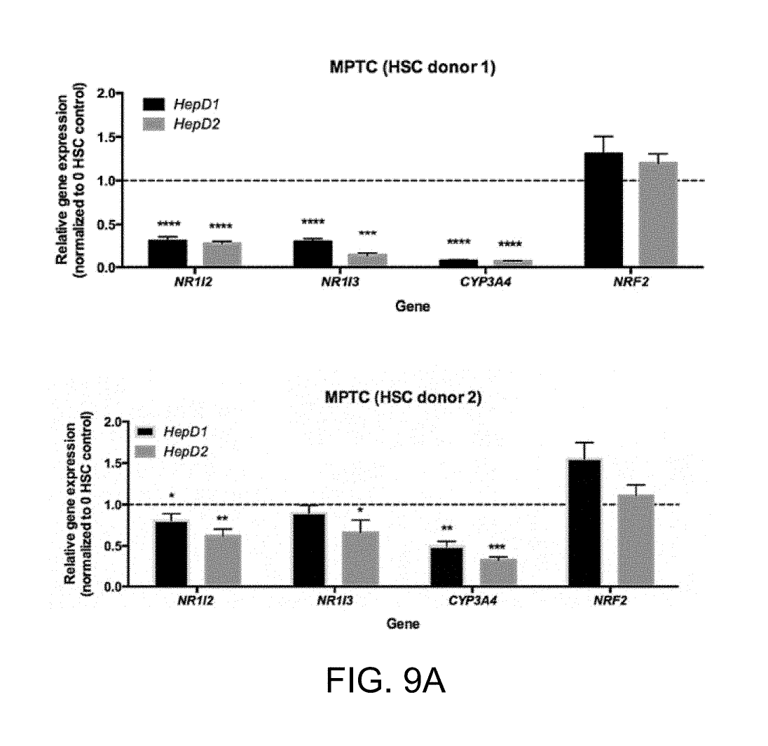

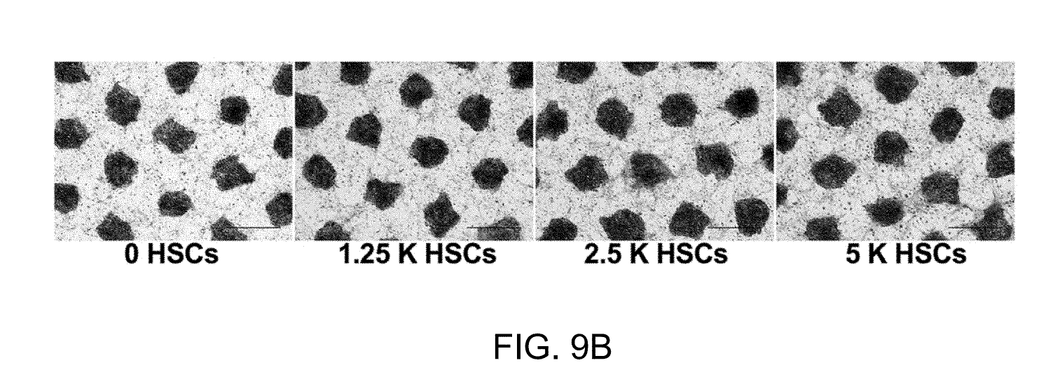

MPTC gene expression for the nuclear receptor pregnane x receptor (NR1I2) and the constitutive androstane receptor (NR113; CAR) showed significant down regulation (FIG. 5F and FIG. 6D) when compared to HSC free MPBCs. CYP3A4 gene expression also showed significant down regulation in MPTCs when compared to MPBCs (FIG. 5F). Hepatocyte nuclear factor 4 α (HNF4α), a master liver transcription factor, gene expression was also decreased in MPTCs when compared to MPBCs (FIG. 5F and FIG. 6C). Hepatocyte nuclear factor 6 (ONECUT1) was not drastically different between the MPBC and MPTC cultures (FIG. 6C). Nuclear factor erythroid 2-like 2 (NRF2), an oxidative stress responsive transcription factor, gene expression was increased in MPTCs, although not significantly (FIG. 5F). Expression of aryl hydrocarbon receptor (AHR) at 2 weeks of culture showed a significant increase in gene expression in the MPTC culture (FIG. 6D). Similar gene expression trends were observed using 2 hepatocyte donors and 2 primary HSC donors (see FIG. 9A). Importantly, this occurred prior to excessive ECM accumulation (see FIG. 9B).

### Example 3. Activated HSCs Decrease Hepatic Transporter Function and Gene Expression

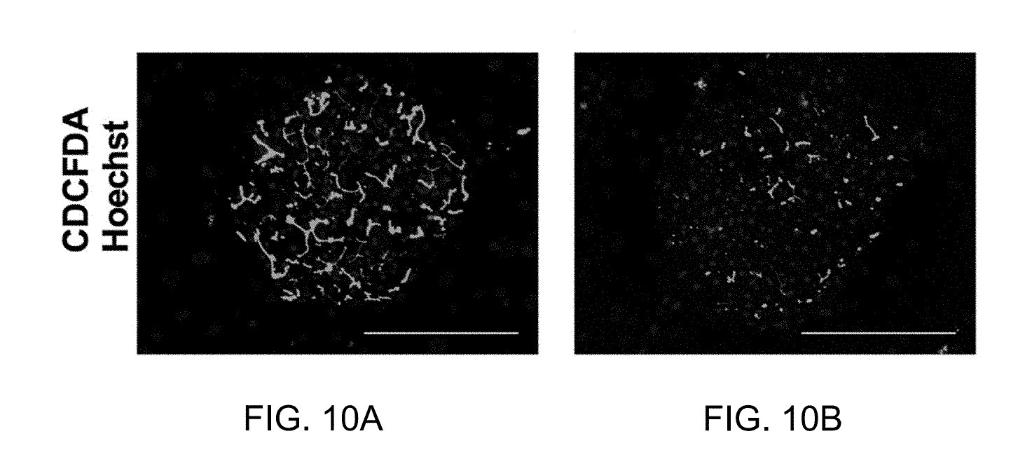

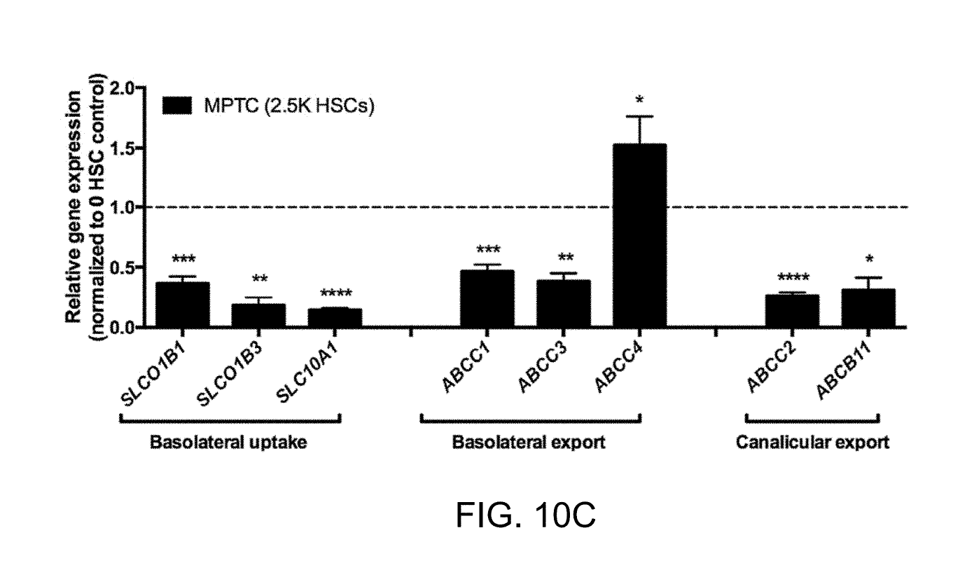

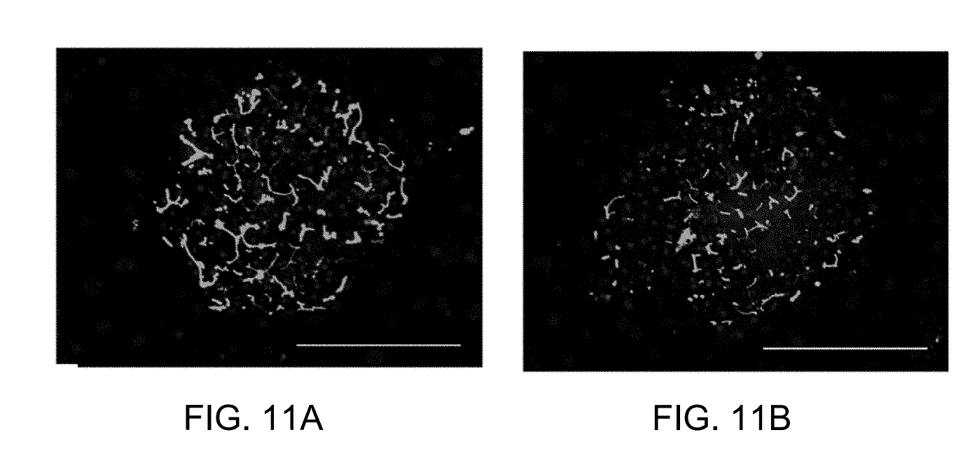

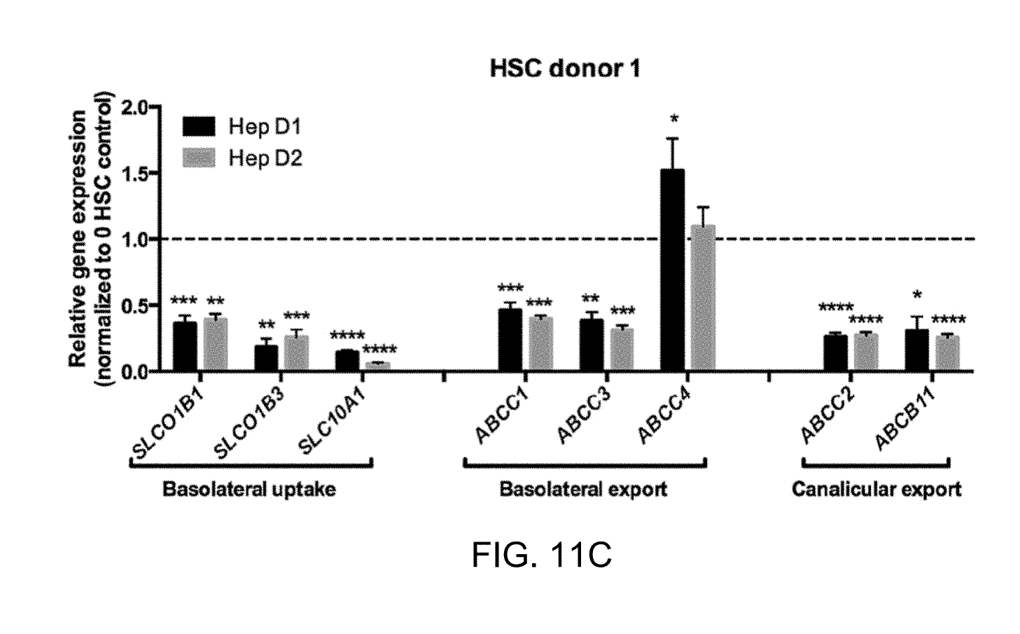

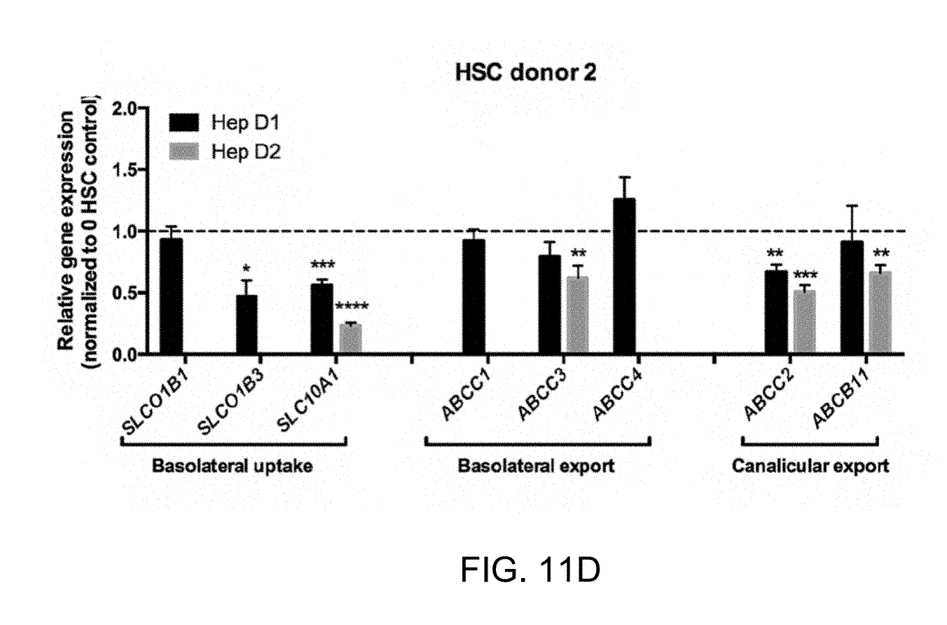

5 (and 6)-carboxy-2′,7′-dichlorofluorescein diacetate (CDCFDA) is a non-fluorescent compound that can passively cross the cell membrane where it is hydrolyzed into a non-membrane permeable fluorescent 5 (and 6)-carboxy-2′,7′-dichlorofluorescein (CDF), via intra-cellular esterases. CDF is then excreted via the basolateral transporter multidrug resistant-like protein 3 (MRP3) and the apical transporter multidrug resistant-like protein 2 (MRP2) (Zamek-Gliszczynski et al. Journal of Pharmacology and Experimental Therapeutics 2003; 304, 801-809). After administering CDCFDA to live MPBCs (FIG. 10A) and MPTCs (FIG. 10B) for 15 minutes, reduced transport of CDF in MPTCs after 2 weeks of culture was observed. Gene expression of basolateral uptake, SLCO1B1 (OATP1B1), SLC01B3 (OATP1B3), and SLC10A1 (NTCP), basolateral export, ABCC1 (MRP1), ABCC3 (MRP3), and ABCC4 (MRP4), and canalicular export transporters, ABCC2 (MRP2), and ABCB11 (BSEP), in MPTCs was significantly lower, except for ABCC4, when compared to MPBC controls (FIG. 10C). Similar trends were observed with transporter function and some of these genes in MPTCs with 2 different hepatocyte donors and stellate cell donors (see FIG. 11).

### Example 4. Hepatic Stellate Cells Cause Hepatocyte Neutral Lipid Accumulation in a Time and Density Dependent Manner

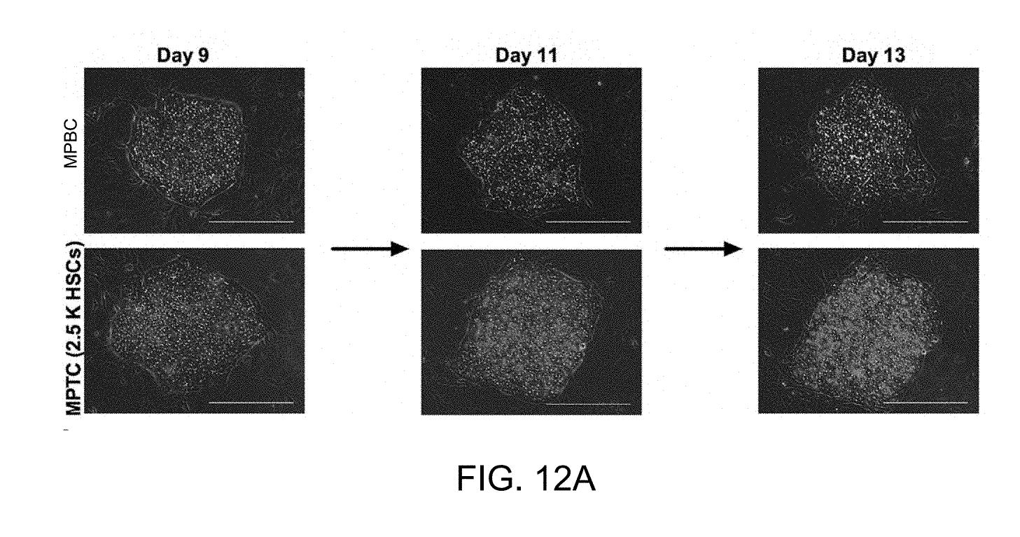

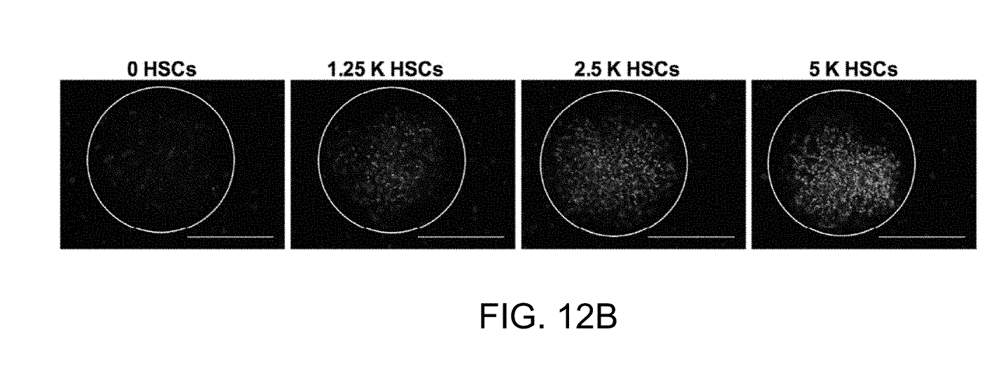

Phase contrast imaging of MPTCs showed noticeable (>5% of hepatocytes/island) lipid accumulation, or steatosis, after 8-9 days in culture (FIG. 12A), which progressed to significant lipid accumulation (>70% of hepatocytes/island) by 13 days of culture (FIG. 12A), whereas MPBCs had minimal lipid accumulation (˜5% of hepatocytes) at 13 days of culture. An HSC density dependent increase in Nile red staining, which fluoresces in the presence of neutral lipids, was also observed in fixed cultures of MPTCs with 1.25, 2.5 and 5 K HSCs seeded initially (FIG. 12B).

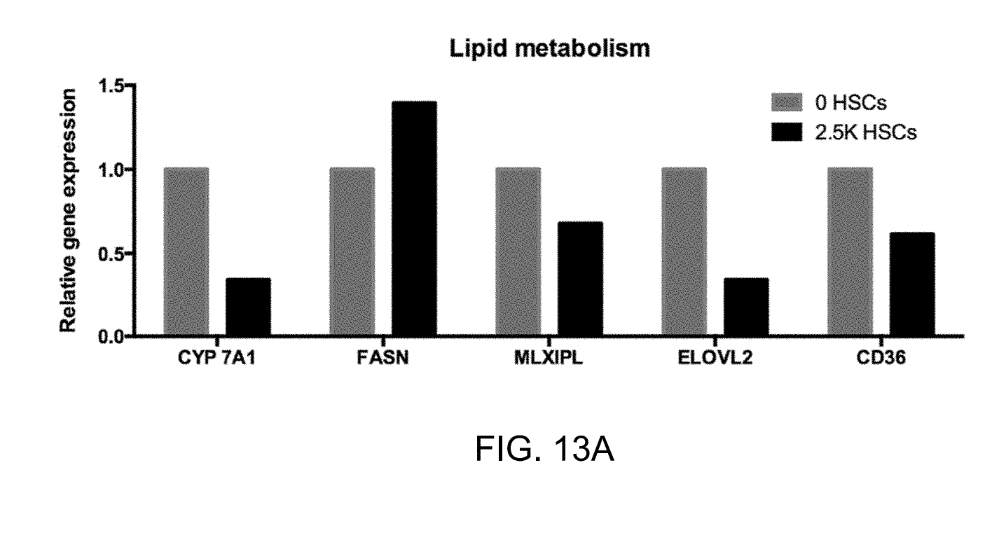

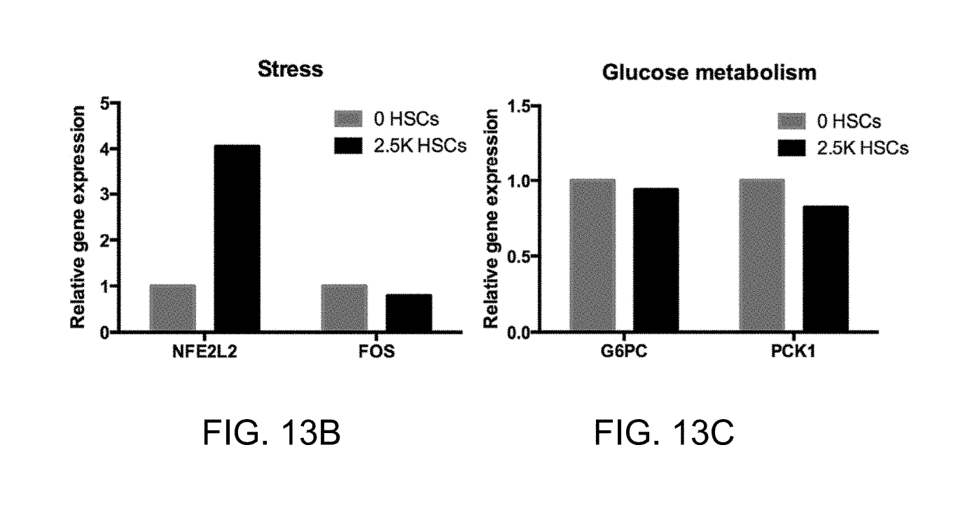

Gene expression analysis showed that activated HSCs alter lipid metabolism and cellular stress response. To determine alterations in lipid metabolism, CYP 7A1, fatty acid synthase (FASN), carbohydrate response element binding protein (ChREBP), ELOVL fatty acid elongase 2 (ELOVL2) and CD-36 gene expression were measured at 2 weeks of culture (FIG. 13A).

Differential expression of the genes evaluated was noted in MPBC cultures versus MPTC cultures. Alterations in cellular stress response were also determined by measuring the gene expression of nuclear factor (erythroid-derived 2)-like 2 (NFE2 L2) and C-FOS (FOS) at 2 weeks of culture (FIG. 13B). A significant upregulation of NFE2L2 was observed in the MPTC cultures. Glucose 6 phosphatase catalytic subunit (G6PC) and phosphoenolpyruvate carboxykinase 1 (PCK1) gene expression was measured at 2 weeks of culture to determine alterations in glucose metabolism (FIG. 13C). No significant difference was observed in either gene in the MPBC cultures relative to the MPTC cultures.

### Example 5. Activated Hepatic Stellate Cell Secreted Interleukin 6 (IL-6) Decreases Hepatocyte CYP 3A4 Activity

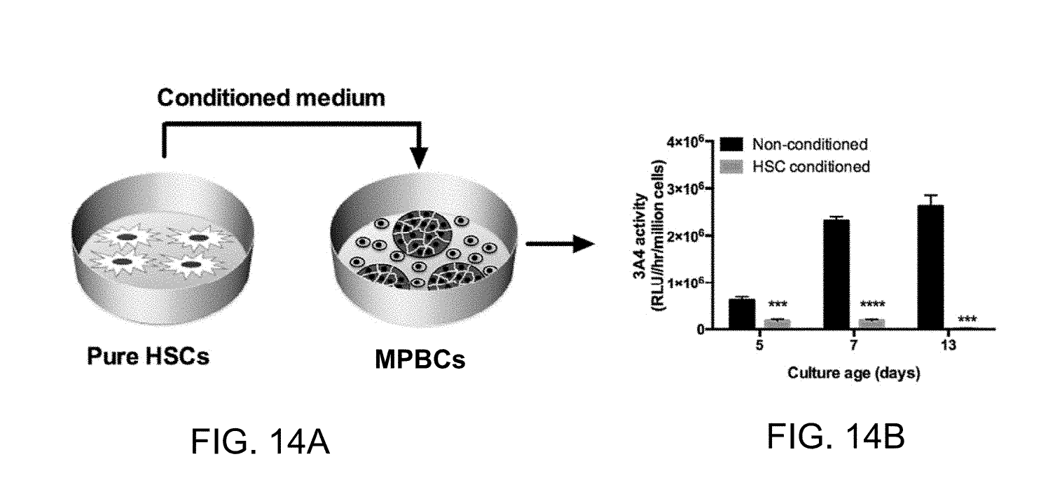

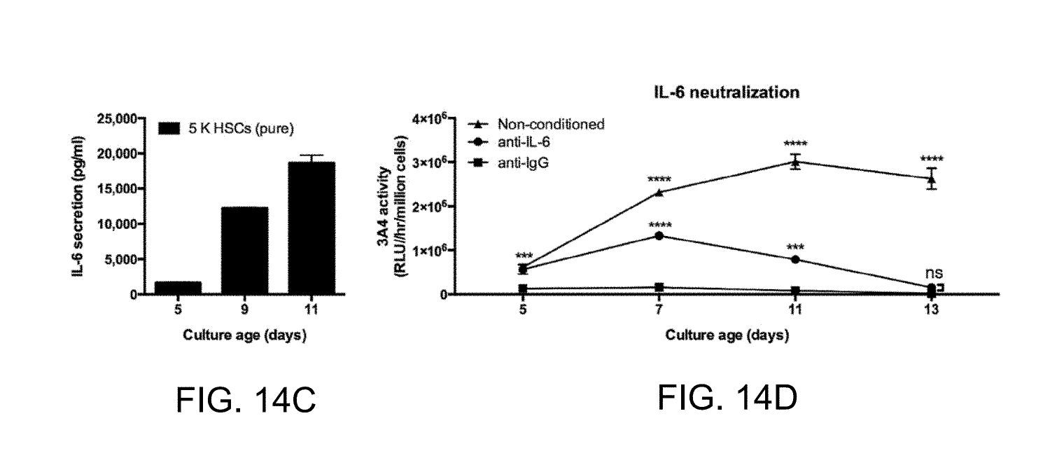

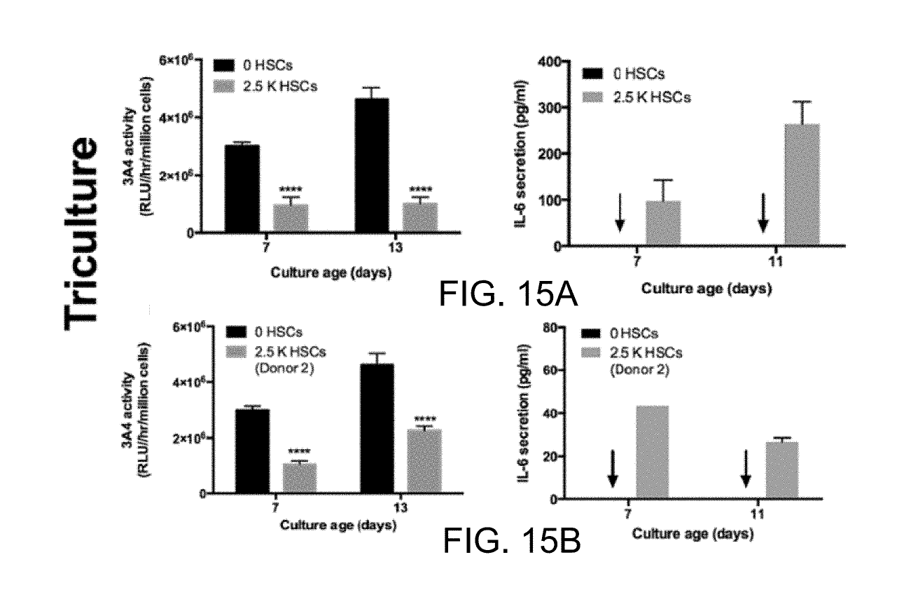

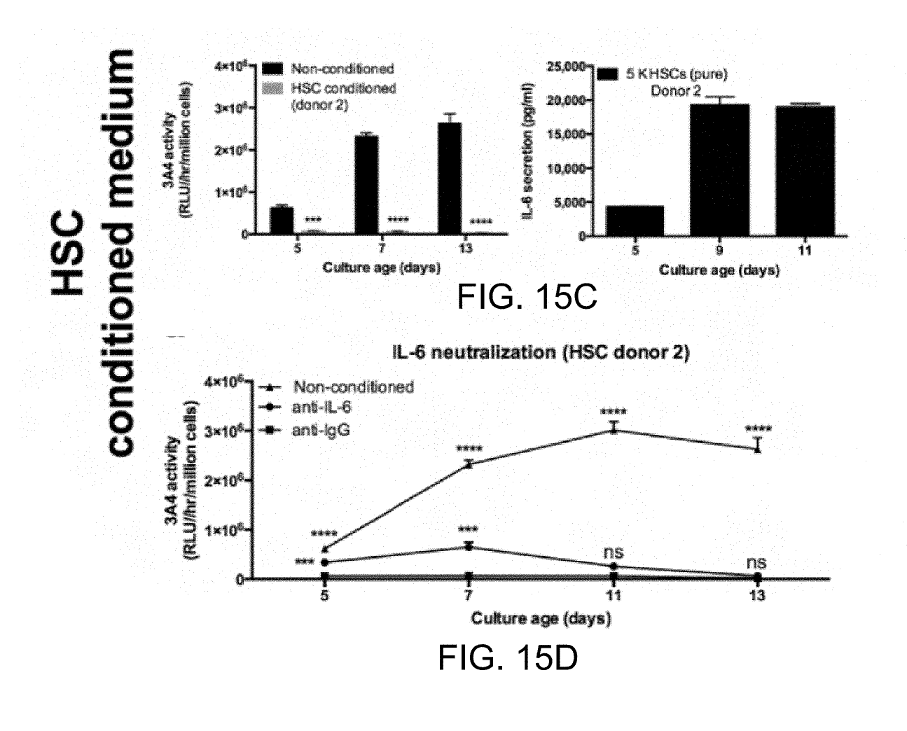

In order to determine the mechanism by which stellate cells induce the changes in hepatocytes, conditioned medium experiments were conducted using pure HSC cultures and MPBCs. Pure HSC culture medium, seeded at 5 K/1.9 cm², was collected, sterile filtered (0.2 μM) and quickly (within ≤1 hour after removal) added to MPBCs, and CYP 3A4 activity was assessed over time (FIG. 14A). CYP 3A4 activity was significantly decreased in MPBCs (FIG. 14B) by conditioning with HSC medium, and this progressively declined with additional days of conditioning. This same trend was also seen with another HSC donor (see FIG. 15C). MPTCs showed similar trends in CYP 3A4 activity at 7 and 13 days (FIGS. 15A, B), although the level of activity was not as dramatically altered as conditioned MPBCs.

Pure HSC supernatant interleukin-6 (IL-6) concentrations were measured using a sandwich ELISA method (R&D Systems, Minneapolis, Minn.) and found to increase over time (FIG. 14C and FIG. 15C), while MPTC IL-6 secretions slightly increased or decreased depending on the HSC donor (see FIGS. 15A, B). Neutralizing antibodies for IL-6 ameliorated the effects of activated HSC conditioned medium on MPBC 3A4 activity during acute exposure (FIG. 14D and FIG. 15), while this effect was lost at later time points.

### Example 6. Stiffness Mediated Hepatic Stellate Cell Activation Alters CYP 3A4 Via IL-6

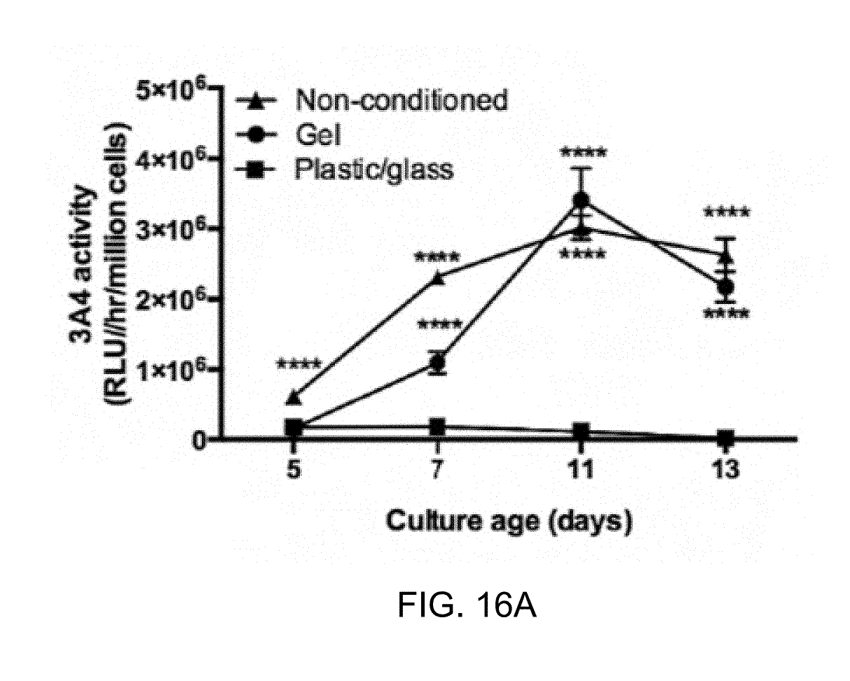

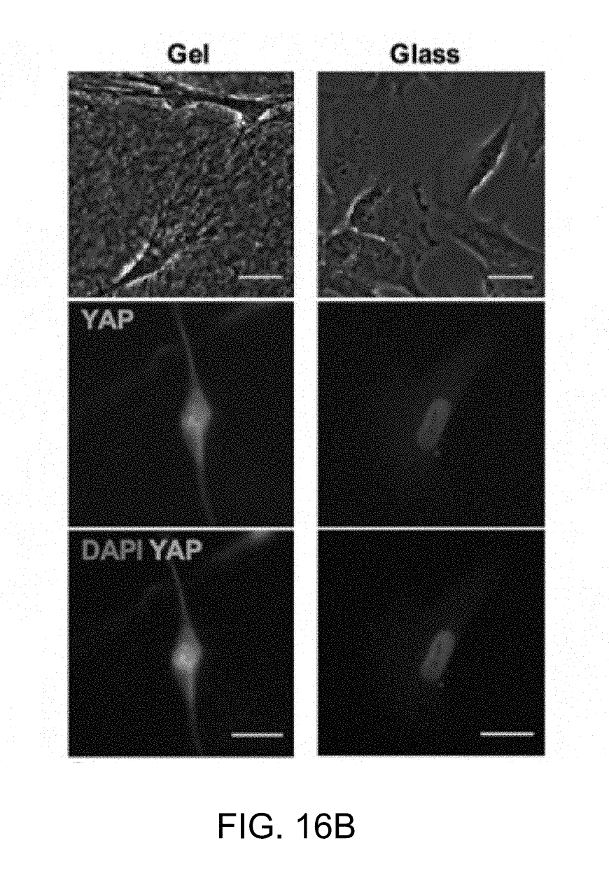

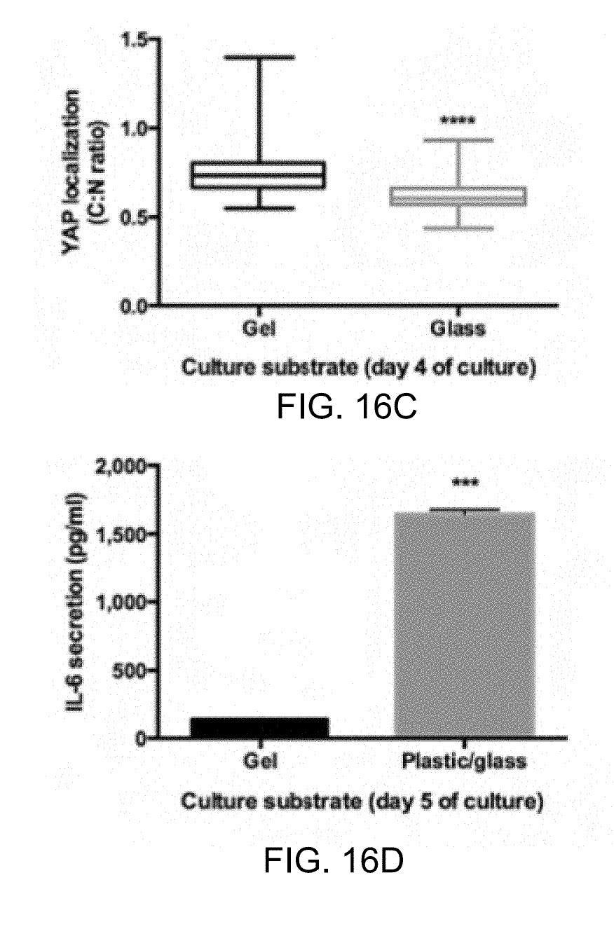

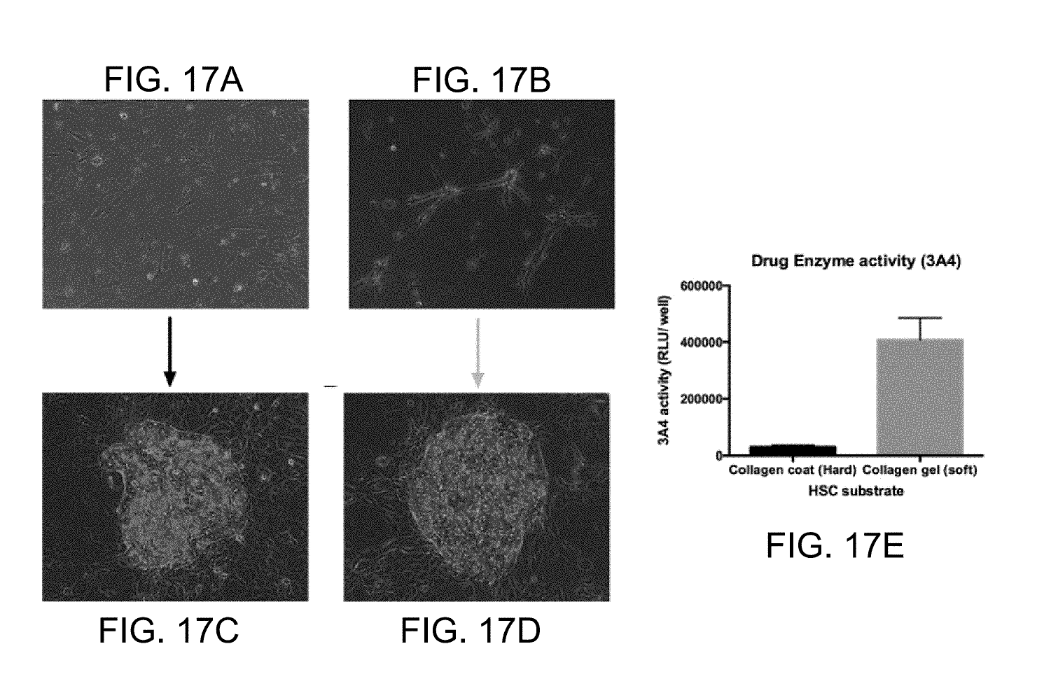

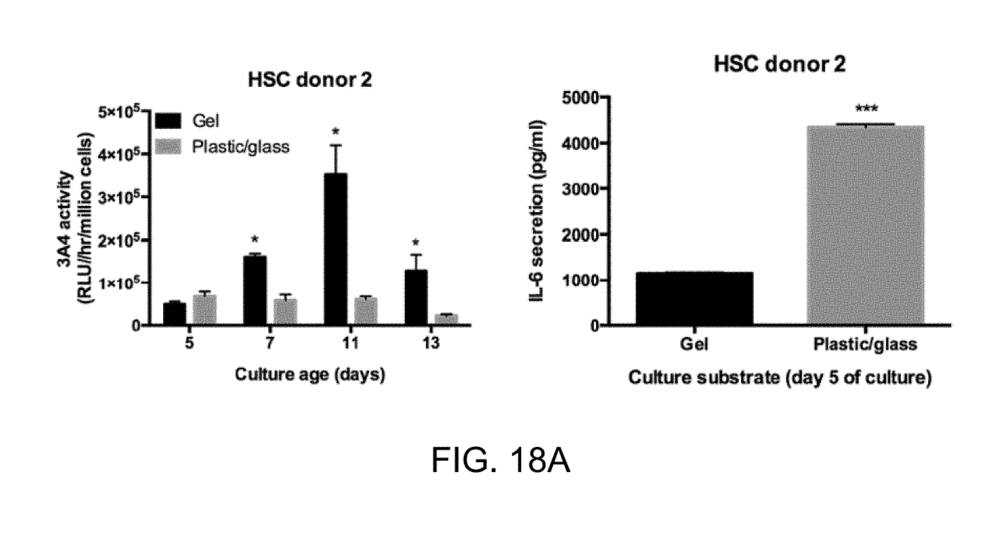

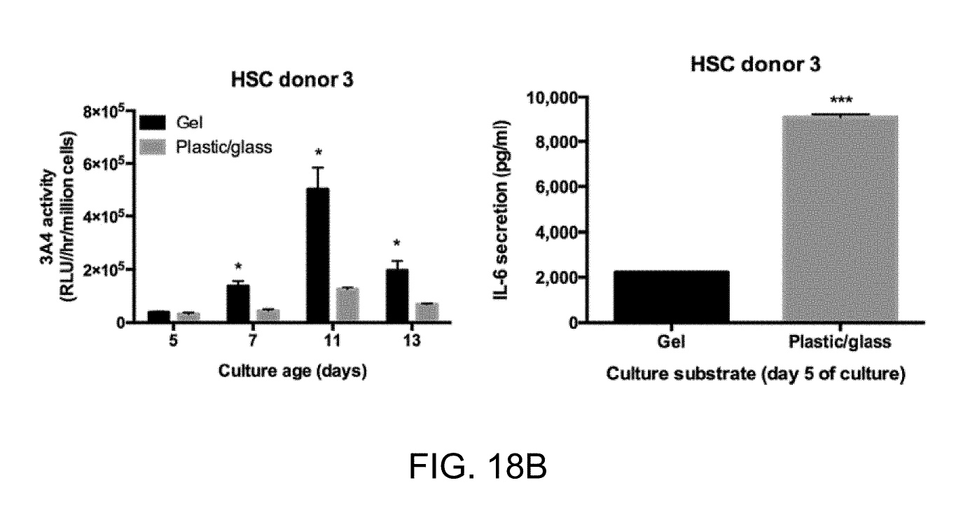

Since stellate cells are activated by increasing substrate stiffness and HSCs in the triculture model reside on stiff plastic, the effects of conditioning MPBCs with medium from HSCs cultured on soft gels was investigated. HSCs were seeded on soft (˜1 KPa) type I collagen gels, as well as collagen coated glass coverslips/plastic at the same density (5 K/1.9 cm²). On day 4, HSCs were fixed and labeled with anti-YAP antibodies and DAPI (FIG. 16B). The cytoplasmic to nuclear (C:N) ratio of YAP was higher in HSCs cultured on gels (FIG. 16C) (n=52 cells/treatment). FIG. 17 demonstrates the HSC morphology on collagen coated plastic (FIG. 17A), 3 mg/ml collagen gel (FIG. 17B), and 2 mg/ml collagen (FIG. 17C) at 2 weeks of culture. IL-6 secretion was significantly higher in HSC cultures (FIG. 16D) on plastic/glass substrates, compared to gel culture secretions. FIG. 17 also demonstrates the micropatterned co-culture morphology after being conditioned for approximately 1 week with medium from HSCs cultured on collagen coated plastic (FIG. 17D), 3 mg/ml collagen gel (FIG. 17E), and 2 mg/ml collagen gel (FIG. 17F). Placing this same HSC conditioned media on MPBCs showed that HSCs on gels do not inhibit CYP 3A4 activity to the same level as HSCs on glass/plastic (FIG. 16A and FIG. 17G). Similar trends were observed with 2 other primary HSC donors, although baseline activity was drastically lower with these 2 donors (see FIGS. 18A, B). Unlike IL-6 neutralization, altering compliance had a lasting effect on rescuing CYP 3A4 activity in MPBCs conditioned with HSC medium (see FIGS. 18A, B). Based on these results, HSC can be cultured with hepatocyte-fibroblast micropatterned co-cultures in an activated state on hard/stiff plastic (which spontaneously activates HSCs) to create a fibrosis model as demonstrated in FIG. 1B. Alternatively, the hepatocyte-fibroblast micropatterned co-culture can be overlaid with a gel (e.g., made of collagen and/or Matrigel) of varying compliances and HSCs cultured on top of the gel to create a liver model in which the HSCs are not activated to the same degree as on plastic, thereby mimicking a liver closer to a healthy state as demonstrated in FIG. 1C.

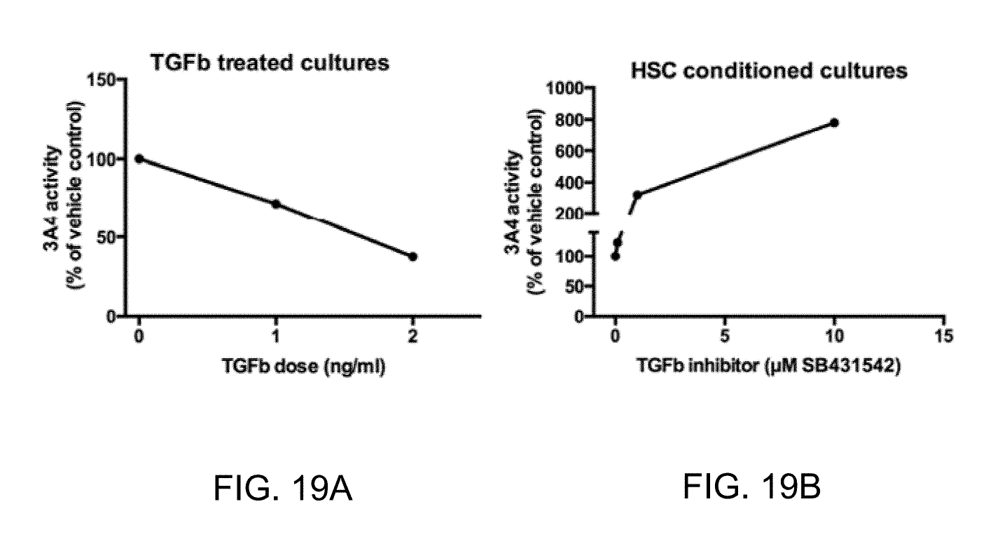

In addition to culturing HSCs on softer substrates to make them more quiescent/less activated, it was evaluated if TGF-beta treatment and TGF-beta receptor inhibition would also alleviate the negative effects of activated HSCs on hepatocyte functions. Hepatocyte-fibroblast micropatterned co-cultures were treated with increasing amounts of TGF-beta growth factor and a dose dependent decrease in 3A4 activity was observed after 10 days of treatment (FIG. 19A). Hepatocyte-fibroblast micropatterned co-cultures were pretreated with the TGF-beta receptor inhibitor SB431542 at increasing doses for 2 hours, then incubated with HSC conditioned medium containing the inhibitor for 10 days, and then 3A4 activity was assessed. A dose dependent increase in 3A4 activity was observed with the addition of the inhibitor (FIG. 19B), which suggests that inhibiting TGF-beta signaling in hepatocytes could ameliorate the negative effects of activated HSCs on hepatocytes.

### Example 7. Retinol and Insulin Prevent and Induce, Respectively, HSC Induced IL-6 Secretion and 3A4 Down Regulation in Hepatocytes

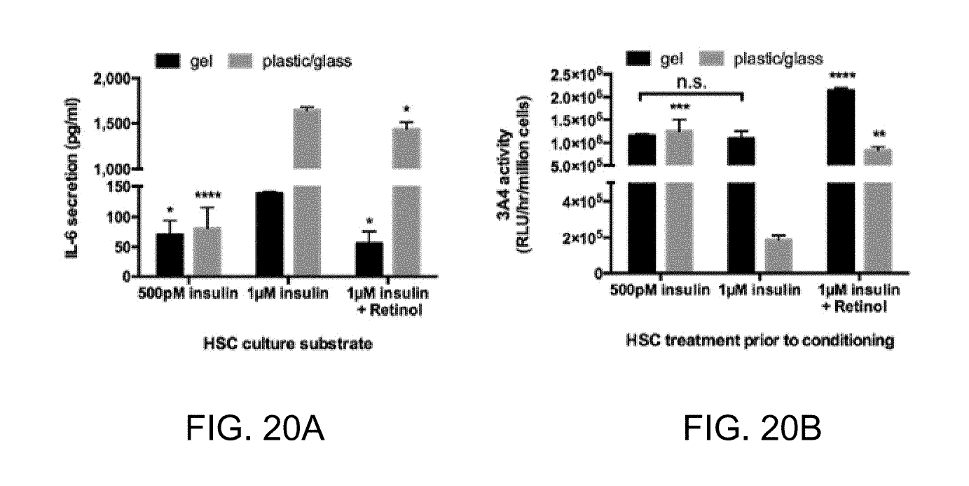

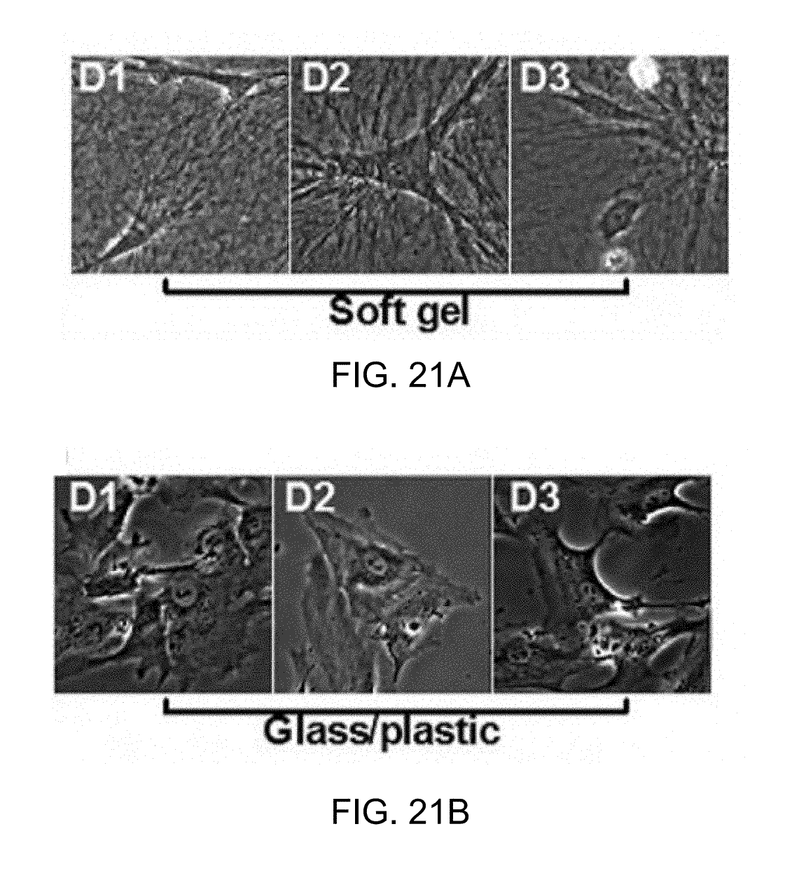

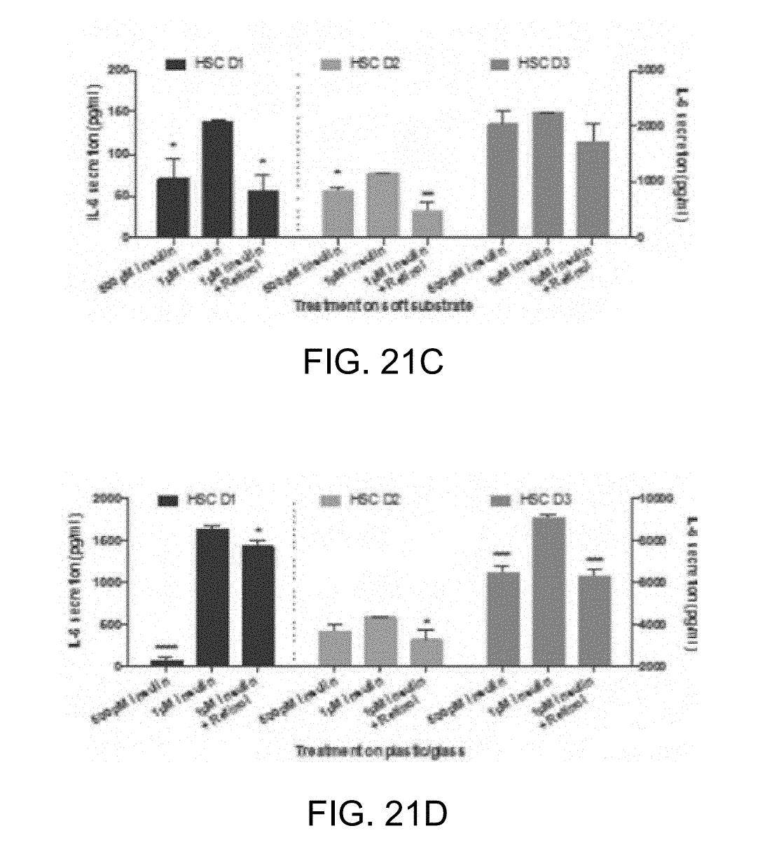

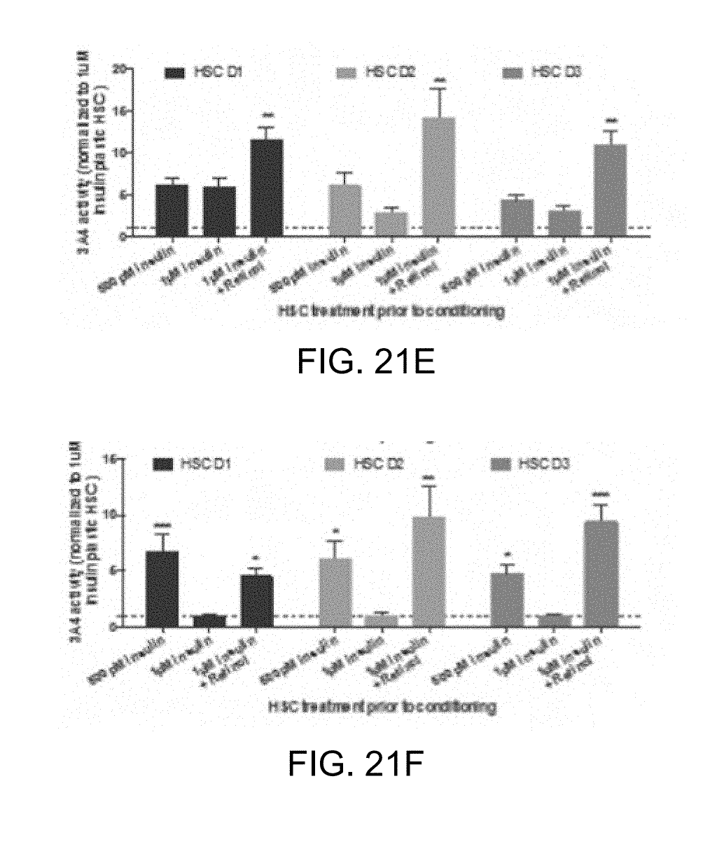

NAFLD onset coincides with a loss of liver vitamin A, and is associated with an increase in blood insulin levels. HSCs cultured on gels displayed significantly lower IL-6 secretion than HSCs on plastic, however the level of CYP 3A4 activity in MPBCs conditioned with this medium was still below the non-conditioned control. Therefore how insulin and retinol might play a role in the development of decreased CYP 3A4 in MPBCs was addressed. HSCs cultured on soft gels, and plastic/glass had significantly decreased IL-6 secretion (FIG. 20A) when treated with low insulin (500 pM) or high insulin (1 μM) plus all-trans retinol (vitamin A) saturated BSA. When this same media was added to MPBCs, low insulin or retinol treated HSC medium had significantly higher CYP 3A4 activity for plastic/glass substrates, while only retinol consistently improved CYP 3A4 activity over high insulin treated HSC cultures on gels (FIG. 20B). Similar trends were observed with 2 other primary HSC donors (see FIG. 21).

### Example 8. Fabrication of Fibrotic and Healthy Paracrine Tri-Culture Models

Paracrine effects (e.g., HSC secretions) can be determined by use of a transwell model (FIG. 2). In the transwell model, transwells are absorbed with extracellular matrix such as collagen or extracellular matrix gels. The HSCs are then seeded onto the matrix and allowed to attach (FIG. 2). Separately, micropatterned co-cultures containing hepatocytes and fibroblasts are established (FIG. 2). The transwells are then transferred to the micropatterned co-cultures (FIG. 2). In contrast to the direct contact model depicted in FIG. 3 and FIG. 1, the hepatocytes and HSCs are not contacting each other but communicating with secreted molecules (paracrine signaling). Such a model can allow identification of secreted HSC factors that negatively affect hepatic functions.

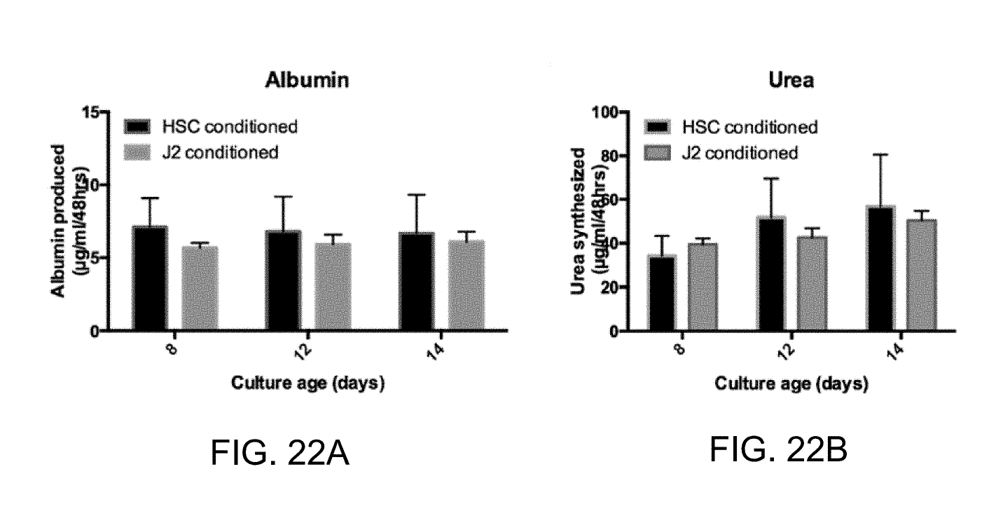

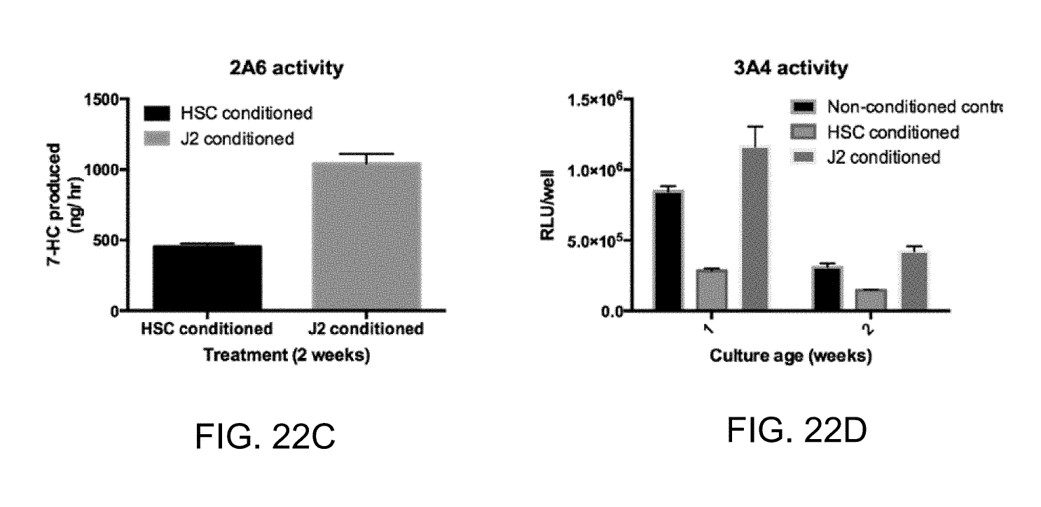

It was assessed whether paracrine factors mediate HSC-hepatocyte interactions. Hepatocyte-fibroblast micropatterned co-cultures, mono-cultures of 3T3-J2 fibroblasts, and co-cultures of HSCs and 3T3-J2 fibroblasts were established simultaneously. Medium from 3T3-J2 mono-cultures or HSC-fibroblast co-cultures was conditioned for 48 hours, sterile filtered (0.2 μm), and then placed on hepatocyte-fibroblast micropatterned co-cultures for an additional 48 hours to assess the effects of paracrine signaling on hepatocyte functions. Albumin and urea production in hepatocyte-fibroblast micropatterned co-cultures conditioned with either 3T3-J2 mono-cultures or HSC-fibroblast co-cultures conditioned medium was not significantly different (FIGS. 22A, B). CYP2A6 and 3A4 activity in hepatocyte-fibroblast micropatterned co-cultures conditioned with either 3T3-J2 mono-cultures or HSC-fibroblast co-cultures conditioned medium was evaluated (FIGS. 22C, D). Note the similar 3A4 activity in 3T3-J2 mono-cultures and non-conditioned medium controls (FIG. 22C). This decrease in hepatocyte 3A4 activity also occurs in pure hepatocyte cultures cultured on gels to sustain phenotype (not shown). This data suggests that functions of hepatocytes in the hepatocyte-fibroblast micropatterned co-cultures were also negatively affected by paracrine secretions from the HSCs, thereby showing that contact between HSCs and hepatocytes is not entirely necessary to mediate the interactions.

### Example 9. Valproic Acid (VPA) Helps Reduce the Negative Effects of HSCs on Hepatocytes

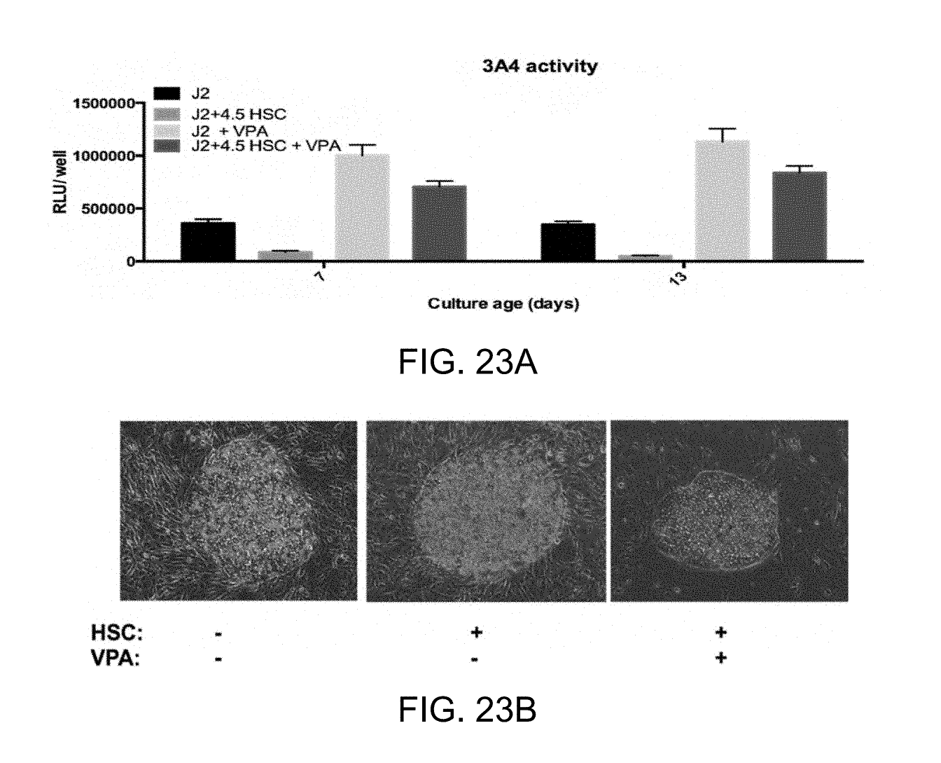

The negative effects of activated HSCs on hepatocyte functions were alleviated by drug treatment (FIG. 23). Valporic acid (VPA) was shown to reduce the negative effects of HSCs on hepatocytes. Cultures were created as shown in FIG. 1B and conditioned with VPA at 2.5 mM for 2 weeks. “J2” condition refers to hepatocyte-fibroblast micropatterned co-cultures while “J2+4.5 HSC” refers to hepatocyte-fibroblast-HSC micropatterned co-cultures with 4,500 HSCs. The addition of VPA resulted in an improvement in hepatocyte morphology as seen in FIG. 23B (compare picture on far right to picture in the middle).

### Example 10. GKT137831 and Obeticholic Acid Administration Alleviates the Detrimental Effects of Activated HSCs on Hepatocytes

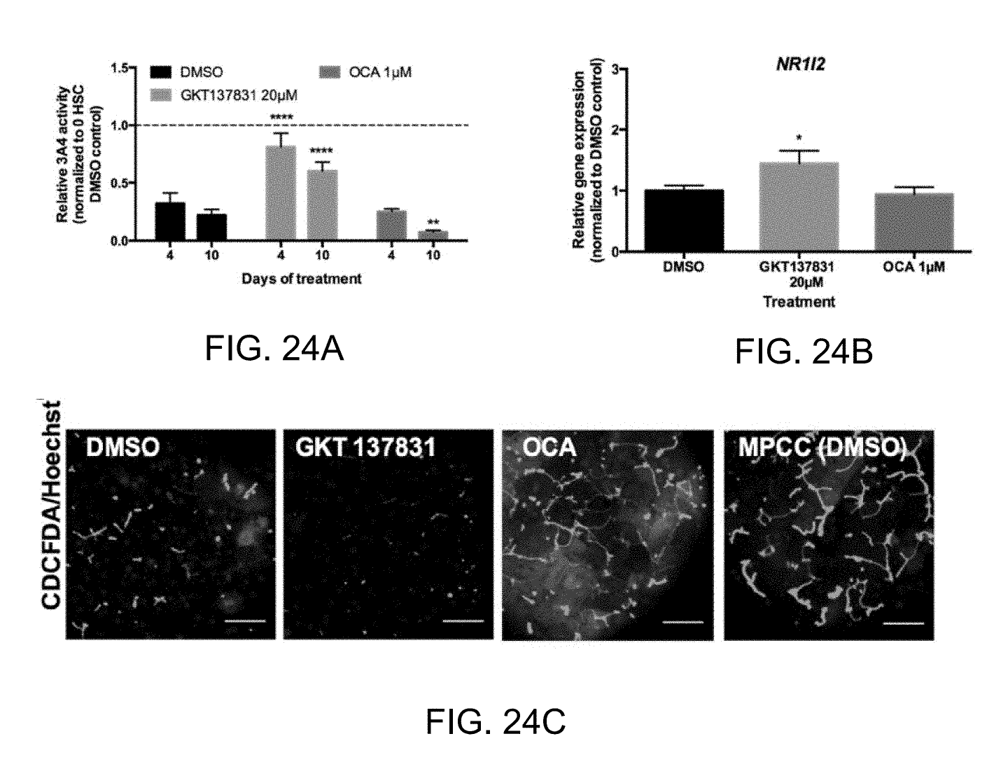

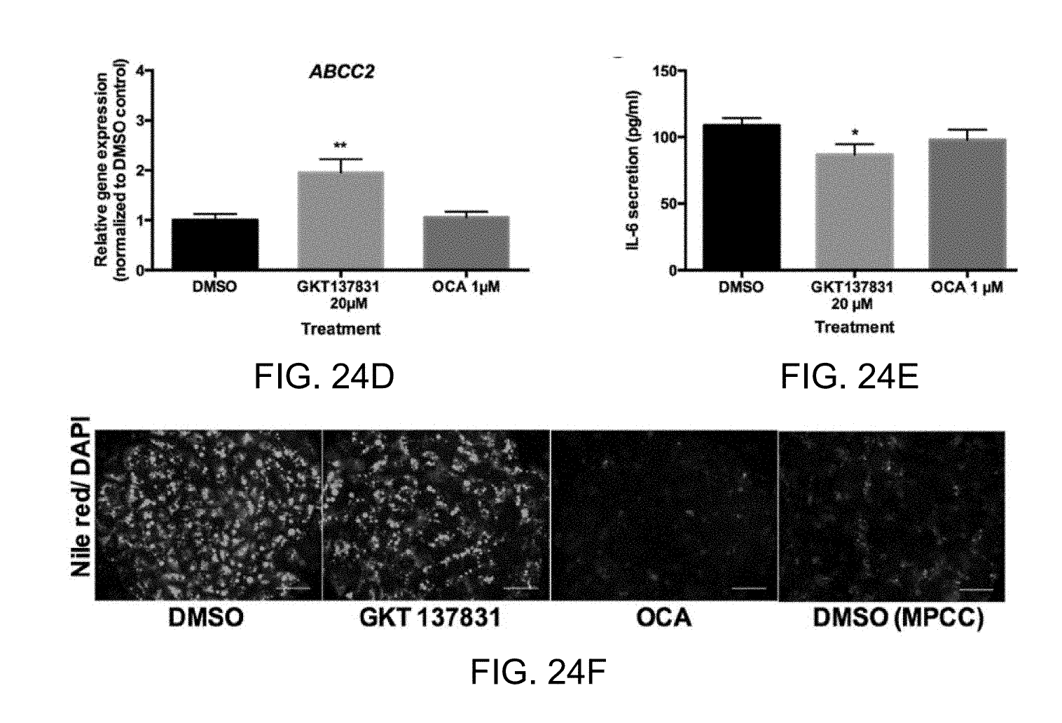

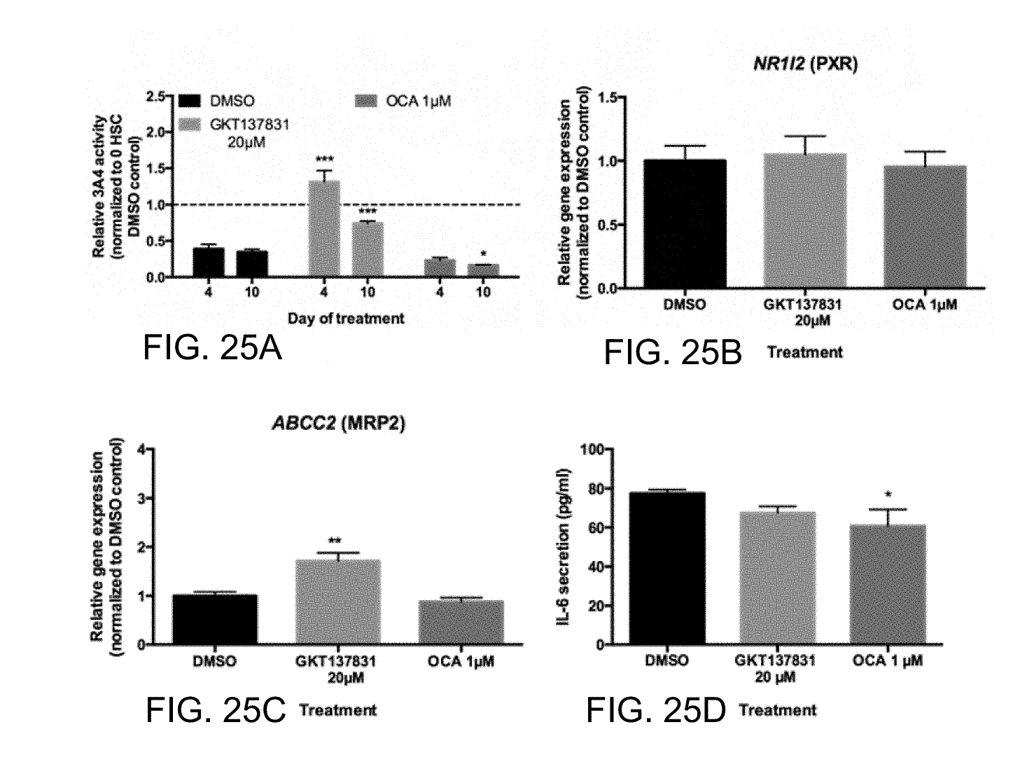

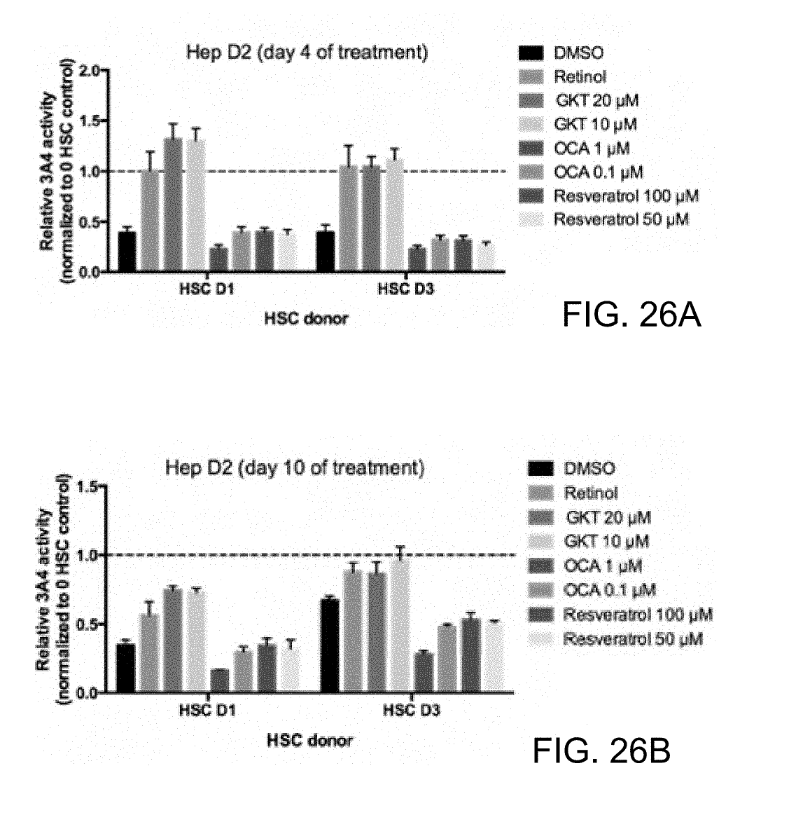

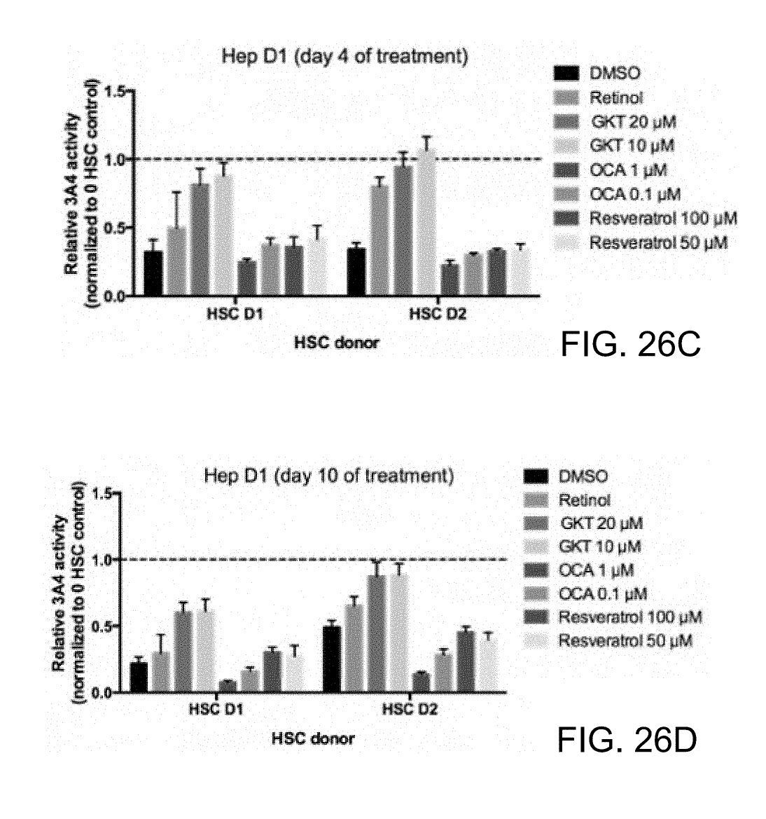

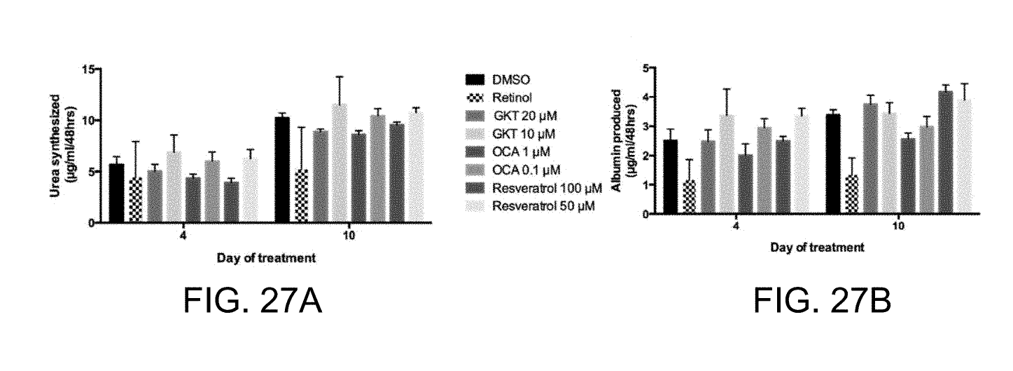

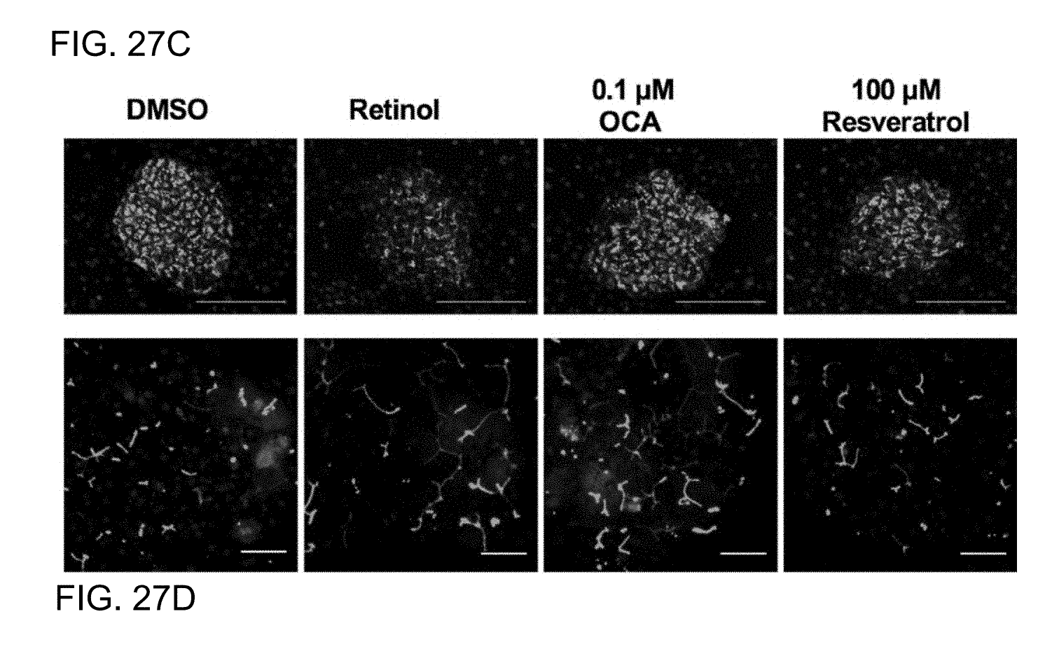

Based on the detrimental effects activated HSCs have on hepatocyte functions and the possible molecular mechanisms that mediate these interactions, it was hypothesized that therapies currently being tested for efficacy in non-alcoholic steatohepatitis (NASH) could aid in alleviating HSCs effects. Initially retinol-BSA, berberine, valproic acid, resveratrol, GKT137831 and obeticholic acid were screened. Berberine and valproic acid were overtly hepatotoxic, at effective dose ranges found in literature, which eliminated these 2 compounds from subsequent more detailed screens. MPTCs were then treated with all of the remaining compounds at 2 different doses and the compound's efficacy was measured by lipid reduction and or rescue of CYP 3A4 activity using 2 hepatocyte donors with 2 different HSC donors (FIGS. 26 A-D). Retinol-BSA and GKT 137831 consistently rescued CYP 3A4 activity over the DMSO treated control (FIG. 24A and FIGS. 26A-D), while retinol-BSA and obeticholic acid improved the lipid accumulation and transporter function (FIG. 26 and FIG. 27). Although, in MPTCs, retinol-BSA was slightly toxic, which limits its potential as a therapeutic option (FIGS. 27 A and B).

The examples above show proof-of-concept of using the tri-culture models developed for discovery and testing of novel therapeutics and for the study of fundamental mechanisms underlying hepatocyte-HSC interactions. Toxicity studies can be coupled with efficacy studies to develop both efficacious and safer drugs using the same platform.

All cited patents and publications are herein incorporated by reference in their entirety.

Whereas particular aspects have been described above for purposes of illustration, it will be appreciated by those skilled in the art that numerous variations of the details may be made without departing form the disclosure as described.

1. A composition comprising a population of hepatocytes, at least one non-parenchymal cell population, and a population of activated hepatic stellate cells in co-culture in vitro.

2. (canceled)

3. The composition according to claim 1, wherein the population of hepatic stellate cells is cultured on a layer of material comprising at least one extracellular matrix protein that is disposed on the population of hepatocytes and at least one non-parenchymal cell population.

4. The composition according to claim 1, wherein the population of hepatic stellate cells is cultured on a substrate that is in fluid communication with the population of hepatocytes and at least one non-parenchymal cell population, and wherein the population of hepatic stellate cells is not in physical contact with the population of hepatocytes and at least one non-parenchymal cell population.

5. The composition according to claim 4, wherein the population of hepatic stellate cells is cultured on a transwell.

6. The composition according to claim 1, wherein the population of hepatic stellate cells is cultured on a hard substrate.

7. A composition comprising a population of hepatocytes and at least one non-parenchymal cell population in co-culture in vitro, and conditioned medium obtained from a population of activated hepatic stellate cells.

8. The composition according to claim 1, wherein the hepatocytes are derived from pluripotent human stem cells.

9. The composition according to claim 1, wherein the hepatocytes are obtained from one or more human donors suffering from a disorder of the liver.

10. The composition according to claim 9, wherein the disorder of the liver is selected from Type-2 diabetes, metabolic syndrome, non-alcoholic fatty liver disease (NAFLD), non-alcoholic steatohepatitis (NASH), and cardiovascular disease.

11. The composition according to claim 1, wherein the hepatic stellate cells are obtained from one or more human donors suffering from a disorder of the liver.

12. The composition of according to claim 11, wherein the disorder of the liver is selected from Type-2 diabetes, metabolic syndrome, non-alcoholic fatty liver disease (NAFLD), non-alcoholic steatohepatitis (NASH), and cardiovascular disease.

13. The composition according to claim 1, wherein the at least one non-parenchymal cell population comprises stromal cells.

14. The composition according to claim 13, wherein the stromal cells are selected from fibroblasts, fibroblast-derived cells, macrophages, endothelial cells, pericytes, inflammatory cells, cholangiocytes and other types of stromal cells, and combinations thereof.

15. A method of identifying a candidate test compound for use in treating a disorder of the liver, the method comprising:
contacting the composition according to claim 1 with the test compound;
maintaining the composition for a time and under conditions sufficient to allow an effect of the test compound on the hepatocytes; and
measuring at least one indicator of hepatic function in the hepatocytes to obtain a test measurement, or applying hepatocyte imagining technology (HIAT) to the hepatocytes to obtain a test image;
and comparing the test measurement to a control measurement from the hepatocytes before contact with the test compound, or the test image to a control image of the hepatocytes before contact with the test compound,
wherein a difference between the test and control is indicative of whether the test compound is a candidate for use in treating a disorder of the liver.

16. The method according to claim 15, wherein the disorder of the liver is selected from Type-2 diabetes, metabolic syndrome, non-alcoholic fatty liver disease (NAFLD), non-alcoholic steatohepatitis (NASH), and cardiovascular disease.

17. A method of determining the toxicity of a test compound, the method comprising:
contacting the composition according to claim 1 with the test compound;
maintaining the composition for a time and under conditions sufficient to allow an effect of the test compound on the hepatocytes; and
measuring at least one indicator of hepatic function in the hepatocytes to obtain a test measurement, or applying hepatocyte imagining technology (HIAT) to the hepatocytes to obtain a test image;
and comparing the test measurement to a control measurement from the hepatocytes before contact with the test compound, or the test image to a control image of the hepatocytes before contact with the test compound,
wherein a difference between the test and control is indicative of hepatotoxicity of the test compound.

18. The method according to claim 15, wherein the at least one indicator of hepatic function is selected from the group consisting of albumin production, urea production, ATP production, glutathione production, enzyme activity, lipid accumulation, liver gene expression, and liver protein expression in the hepatocytes.

19-30. (canceled)

31. The method of according claim 15, wherein at least one indicator of hepatic stellate cell function is measured.

32. A method of identifying a candidate test compound for use in treating a disorder of the liver, the method comprising:
contacting the composition according to claim 7 with the test compound;
maintaining the composition for a time and under conditions sufficient to allow an effect of the test compound on the hepatocytes; and
measuring at least one indicator of hepatic function in the hepatocytes to obtain a test measurement, or applying hepatocyte imagining technology (HIAT) to the hepatocytes to obtain a test image;
and comparing the test measurement to a control measurement from the hepatocytes before contact with the test compound, or the test image to a control image of the hepatocytes before contact with the test compound,
wherein a difference between the test and control is indicative of whether the test compound is a candidate for use in treating a disorder of the liver.

33. The method according to claim 32, wherein the disorder of the liver is selected from Type-2 diabetes, metabolic syndrome, non-alcoholic fatty liver disease (NAFLD), non-alcoholic steatohepatitis (NASH), and cardiovascular disease.

34. A method of determining the hepatotoxicity of a test compound, the method comprising:
contacting the composition according to claim 7 with the test compound;
maintaining the composition for a time and under conditions sufficient to allow an effect of the test compound on the hepatocytes; and
measuring at least one indicator of hepatic function in the hepatocytes to obtain a test measurement, or applying hepatocyte imagining technology (HIAT) to the hepatocytes to obtain a test image;
and comparing the test measurement to a control measurement from the hepatocytes before contact with the test compound, or the test image to a control image of the hepatocytes before contact with the test compound,
wherein a difference between the test and control is indicative of hepatotoxicity of the test compound.

35. The method according to claim 32, wherein the at least one indicator of hepatic function is selected from the group consisting of albumin production, urea production, ATP production, glutathione production, enzyme activity, lipid accumulation, liver gene expression, and liver protein expression in the hepatocytes.

36-42. (canceled)

43. A method of culturing a population of hepatocytes in vitro comprising: co-culturing the population of hepatocytes with at least one non-parenchymal cell population and a population of activated hepatic stellate cells.

44. A method of culturing a population of hepatocytes in vitro comprising: co-culturing the population of hepatocytes with at least one non-parenchymal cell population and providing conditioned medium obtained from a population of activated hepatic stellate cells.
